(19) United States
(12) Patent Application Publication
(10) Pub. No.: US 2009/0098533 A1
(43) Pub. Date: Apr. 16, 2009

(54) Methods and kits for investigating cancer

(76) Inventors: Marc Munnes, Erkrath (DE); Hans Bojar, Langenfeld (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/561,485

(22) PCT Filed: Oct. 2, 2004
(86) PCT No.: PCT/EP04/11009

§ 371 Date: Aug. 28, 2006

(30) Foreign Application Priority Data
Oct. 6, 2003 (EP) 03022587.4
Jun. 30, 2004 (EP) 04015374.4

(51) Int. Cl. C12Q 1/68; G06F 19/00

(52) U.S. Cl. 435/6; 702/20

(57) ABSTRACT

The invention provides novel compositions, methods and uses, for the prediction, diagnosis, prognosis, prevention and treatment of malignant neoplasia and breast cancer. The invention further relates to genes that are differentially expressed in breast tissue of breast cancer patients versus those of normal “healthy” tissue. Differentially expressed genes for the identification of patients which are likely to respond to chemotherapy are also provided.

### TECHNICAL FIELD OF THE INVENTION

The present invention relates to methods and compositions for the prediction of therapy outcome (e.g. tumor response to therapy), diagnosis, prognosis, prevention and treatment of neoplastic diseases. Cancer cells display a specific pattern of gene expression related to their morphological type, state of progression, acquirement of genomic alterations, point mutations in critical genes such as gatekeepers and tumor suppressors or due to the dependency of external signals such as growth factors, hormones or other secondary messengers.

The invention discloses genes which show an altered expression in a particular neoplastic tissue compared to the corresponding healthy tissue or to other neoplastic lesions unresponsive to a given chemotherapy. They are useful as diagnostic markers and could be also regarded as therapeutically targets. Methods are disclosed for predicting, diagnosing and prognosing as well as preventing and treating neoplastic disease. The genes disclosed in this invention have been identified in breast cancers but are predictable of outcome to a certain therapy regimen and therefor they are also relevant for other types of cancers in tissues other than breast.

### BACKGROUND OF THE INVENTION AND PRIOR ART

Cancer is the second leading cause of death in the United States after cardiovascular disease. One in three Americans will develop cancer in his or her lifetime, and one of every four Americans will die of cancer. More specifically breast cancer claims the lives of approximately 40,000 women and is diagnosed in approximately 200,000 women annually in the United States alone. Cancer are classified based on different parameters, such as tumor size, invasion status, involvement of lymph notes, metastasis, histolopathology, immunohistochemical markers, and molecular markers (WHO. International Classification of diseases (1); Sabin and Wittekind, 1997 (2)). With the recent advances in gene chip technology, researchers are increasingly focusing on the categorization of tumors based on the distinct expression of marker genes Sorlie et al., 2001 (3): van 't Veer et al., 2002 (4).

Chemotherapy remains a mainstay in therapeutic regimens offered to patients with breast cancer, particularly those who have cancer that has metastasized from its site of origin (Perez, 1999, (5)). There are several chemotherapeutic agents that have demonstrated activity in the treatment of breast cancer and research is continuously in an attempt to determine optimal drugs and regimens. However, different patients tend to respond differently to the same therapeutic regimen. Currently, the individuals response to certain therapy can only be assessed statistically, based on data of former clinical studies. There are still a great number of patients who will not benefit from a systemic chemotherapy. Especially, breast cancers are very heterogeneous in their aggressiveness and treatment response. They contain different genetic mutations and variations affecting growths characteristic and sensitivity to several drugs. Identification of each tumor's molecular fingerprint, then, could help to segregate patients who have particularly aggressive tumors or who need to be treated with specific beneficial therapies. As research involving genetics and associated responses to treatment matures, standard practice will undoubtedly become more individualized, enabling physicians to provide specific treatment regimens matched with a tumor's genetic profiles to ensure optimal outcomes.

### SUMMARY OF THE INVENTION

The present invention relates to the identification of 185 human genes being differentially expressed in neoplastic tissue resulting in an altered clinical behavior of a neoplastic lesion. The differential expression of these 185 genes is not limited to a specific neoplastic lesion in a certain tissue of the human body.

In preferred embodiments of this invention the neoplastic lesion, of which these 185 genes are altered in their expression is a cancer of the human breast. This cancer is not limited to females and may also be diagnosed and analyzed in males.

The invention relates to various methods, reagents and kits for diagnosing, staging, prognosis, monitoring and therapy of breast cancer. “Breast cancer” as used herein includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The compositions, methods, and kits of the present invention comprise comparing the level of mRNA expression of a single or plurality (e.g. 2, 5, 10, or 50 or more) of genes (hereinafter “marker genes”, listed in Table 1a and 1b, SEQ ID NO:1 to 165 and 472 to 491, the respective polypeptide sequences coded by them are numerated SEQ ID NO: 166 to 330 and 492 to 511, see also Table 1a and 1b) in a patient sample, and the average level of expression of the marker gene(s) in a sample from a control subject (e.g., a human subject without breast cancer). A preferred sub-set of marker genes representing a specific test composition or kit is listed in Table 2.

The invention relates further to various compositions, methods, reagents and kits, for prediction of clinically measurable tumor therapy response to a given breast cancer therapy. The compositions, methods, and kits of the present invention comprise comparing the level of mRNA expression of a single or plurality (e.g. 2, 5, 10, or 50 or more) of breast cancer marker genes in an unclassified patient sample, and the average level of expression of the marker gene(s) in a sample cohort comprising patient responding in different intensity to an administered breast cancer therapy. In preferred embodiments of this invention the specific expression of the marker genes can be utilized for discrimination of responders and non-responders to an anthracycline based (e.g. polychemotherapies with epirubicin or doxorubicin) chemo-therapeutic intervention.

In further preferred embodiments, the control level of mRNA expression is the average level of expression of the marker gene(s) in samples from several (e.g., 2, 3, 4, 5, 8, 10, 12, 15, 20, 30 or 50) control subjects. These control subjects may either be not affected by breast cancer or be identified and classified by their clinical response prior to the determination of their individual expression profile.

As elaborated below, a significant change in the level of expression of one or more of the marker genes (set of marker genes) in the patient sample relative to the control level provides significant information regarding the patient's breast cancer status and responsiveness to chemotherapy. In the compositions, methods, and kits of the present invention the marker genes listed in Table 1a and 1b may also be used in combination with well known breast cancer marker genes (e.g. CEA, mammaglobin, or CA 15-3)

According to the invention, the marker gene(s) and marker gene sets are selected such that the positive predictive value of the compositions, methods, and kits of the invention is at least about 10%, preferably about 25%, more preferably about 50% and most preferably about 90%. Also preferred for use in the compositions, methods, and kits of the invention are marker gene(s) and sets that are differentially expressed, as compared to normal breast cells, by at least the minimal mean differential expression factor presented in Table 3, in at least about 20%, more preferably about 50% and most preferably about 75% of any of the following conditions: stage 0 breast cancer patients, stage I breast cancer patients, stage II breast cancer patients, stage III breast cancer patients, stage IV breast cancer patients, grade I breast cancer patients, grade II breast cancer patients, grade III breast cancer patients, malignant breast cancer patients, patients with primary carcinomas of the breast, and all other types of cancers, malignancies and transformations associated with the breast.

The detection of marker gene expression is not limited to the detection within a primary, secondary or metastatic lesion of breast cancer patients, and may also be detected in lymph nodes affected by breast cancer cells or minimal residual disease cells either locally deposited (e.g. bone marrow, liver, kidney) or freely floating throughout the patients body.

In one embodiment of the compositions, methods, reagents and kits of the present invention, the sample to be analyzed is tissue material from neoplastic lesion taken by aspiration or punctuation, excision or by any other surgical method leading to biopsy or resected cellular material. In one embodiment of the compositions, methods, and kits of the present invention, the sample comprises cells obtained from the patient. The cells may be found in a breast cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

In accordance with the compositions, methods, and kits of the present invention the determination of gene expression is not limited to any specific method or to the detection of mRNA. The presence and/or level of expression of the marker gene in a sample can be assessed, for example, by measuring and/or quantifying of:
1) a protein encoded by the marker gene in Table 1a and 1b (SEQ ID NO:1 to 165 and 472 to 491) or a polypeptide comprising a polypeptide selected from SEQ ID NO:166 to 330 and 492 to 511 or a polypeptide resulting from processing or degradation of the protein (e.g. using a reagent, such as an antibody, an antibody derivative, or an antibody fragment, which binds specifically with the protein or polypeptide)
2) a metabolite which is produced directly (i.e., catalyzed) or indirectly by a protein encoded by the marker gene in Table 1a and 1b (SEQ ID NO:1 to 165 and 472 to 491) or by a polypeptide comprising a polypeptide selected from SEQ ID NO:166 to 330 and 492 to 511
3) a RNA transcript (e.g., mRNA, hnRNA) encoded by the marker gene in Table 1a and 1b, or a fragment of the RNA transcript (e.g. by contacting a mixture of RNA transcripts obtained from the sample or cDNA prepared from the transcripts with a substrate having nucleic acid comprising a sequence of one or more of the marker genes listed within Table 1a and 1b fixed thereto at selected positions). The mRNA expression of these genes can be detected e.g. with DNA-microarrays as provided by Affymetrix Inc. or other manufacturers. U.S. Pat. No. 5,556,752. In a further embodiment the expression of these genes can be detected with bead based direct fluorescent readout techniques such as provided by Luminex Inc. PCT No. WO 97/14028.

In one aspect, the present invention provides a composition, method, and kit of assessing whether a patient is afflicted with breast cancer (e.g., new detection or “screening”, detection of recurrence, reflex testing, especially in patients having an enhanced risk of developing breast cancer (e.g., patients having a familial history of breast cancer and patients identified as having a mutant oncogene). For this purpose the composition, method, and kit comprises comparing:
a) the level of expression of a single or plurality of marker genes in a patient sample, wherein at least one (e.g. 2, 5, 10, or 50 or more) of the marker genes is selected from the marker genes of Table 1a and 1b and
b) the normal level of expression of the marker gene in a control subject without breast cancer.

A significant increase as well as decrease in the level of expression of the selected marker genes (e.g. 2, 5, 10, or 50 or more) in the patient sample relative to each marker gene's normal level of expression is an indication that the patient is afflicted with breast cancer.

The composition, method, and kit of the present invention is also useful for prognosing the progression or the outcome of the malignant neoplasia. For this purpose the composition, method, and kit comprises comparing
a) the level of expression of a single or plurality of marker genes in a patient sample, wherein at least one (e.g. 2, 5, 10, or 50 or more) of the marker genes is selected from the marker genes of Table 1a and 1b
b) a control pattern of expression of these marker genes.

The composition, method, and kit of the present invention is particularly useful for identifying patients who will respond to a certain chemotherapy. For this purpose the composition, method, and kit comprises comparing
a) the level of expression of a single or plurality of marker genes in a patient sample, wherein at least one (e.g. 2, 5, 10, or 50 or more) of the marker genes is selected from the marker genes of Table 1a and 1b and
b) the level of expression of the marker gene in a control subject. The control subject may either be not affected by breast cancer or be identified and classified by their clinical response to the particular chemotherapy.

In another aspect, the invention provides a composition, method, and kit of assessing the efficacy of a therapy for inhibiting breast cancer in a patient. This composition, method, and kit comprises comparing:
a) expression of a single or plurality of marker genes in a first sample obtained from the patient prior to any treatment of the patient, wherein at least one of the marker genes is selected from the marker genes listed within Table 1a and 1b and
b) expression of the marker gene in a second sample obtained from the patient following at least one dose of the therapy.

It will be appreciated that in this composition, method, and kit the “therapy” may be any therapy for treating breast cancer including, but not limited to, chemotherapy, anti-hormonal therapy, directed antibody therapy, radiation therapy and surgical removal of tissue, e.g., a breast tumor. Thus, the compositions, methods, and kits of the invention may be used to evaluate a patient before, during and after therapy, for example, to evaluate the reduction in tumor burden.

In a further aspect, the present invention provides a composition, method, and kit for monitoring the progression of breast cancer in a patient. This composition, method, and kit comprising:
a) detecting in a patient sample at a first time point, the expression of a single or plurality of marker genes, wherein at least one of the marker genes is selected from the marker genes listed in Table 1a and 1b
b) repeating step a) at a subsequent time point in time; and
c) comparing the level of expression of each marker gene detected in steps a) and b), and therefrom monitoring the progression of breast cancer in the patient.

In another aspect, the invention provides a composition, method, and kit for in vitro selection of a therapy regime (e.g. the kind of chemotherapeutical argents) for inhibiting breast cancer in a patient. This composition, method, and kit comprises the steps of:
a) obtaining a sample comprising cancer cells from the patient;
b) separately maintaining aliquots of the sample in the presence of a diverse test compositions;
c) comparing expression of a single or plurality of marker genes, selected from the marker genes listed in Table 1a and 1b;
   in each of the aliquots; and
d) selecting one of the test compositions which induces a lower level of expression of genes from SEQ ID 11, 17, 22, 25, 31, 36, 48, 49, 57, 83, 107, 108, 112, and 159 and/or a higher level of expression of genes from SEQ ID 24, 47, 54, 58, 59, 60, 67, 79, 80, 88, 114, 118, 135, and 141 in the aliquot containing that test composition, relative to the level of expression of each marker gene in the aliquots containing the other test compositions.

The invention further provides a composition, method, and kit of assessing the carcinogenic potential of a certain biological or chemical compound. This composition, method, and kit comprises the steps of:
a) maintaining separate aliquots of breast cells in the presence and absence of the test compound; and
b) comparing expression of a singe or plurality of marker genes in each of the aliquots, wherein at least one of the genes is selected from the marker genes listed within Table 1a and 1b, A significant increase in the level of expression of genes from SEQ ID 19, 23, 36, 45, 62, 74, 81, 96, 103, 106, 107, 112, 113, and 132 and/or a significant decrease of genes from SEQ ID 22, 25, 31, 40, 43, 47, 55, 57, 59, 60, 108, 119, 121, 124, 154, 156, 157, 158, 159, 160, 162, and 164 in the aliquot maintained in the presence of (or exposed to) the test compound, relative to the level of expression of each marker gene in the aliquot maintained in the absence of the test compound, is an indication that the test compound possesses breast carcinogenic potential.

The invention further provides a composition, method, and kit of treating a patient afflicted with breast cancer. This composition, method, and kit comprises providing to cells of the patient an antisense oligonucleotide complementary to a polynucleotide sequence of a marker gene listed within Table 1a and 1b

The invention additionally provides a composition, method, and kit of inhibiting breast cancer cells in a patient at risk for developing breast cancer. This composition, method, and kit comprises inhibiting expression of a marker gene listed in Table 1a and 1b.

In yet another embodiment the invention provides compositions, methods, and kits of screening for agents which regulate the activity of a polypeptide comprising a polypeptide selected from SEQ ID NO: 166 to 330 and 492 to 511. A test compound is contacted with the particular polypeptide. Binding of the test compound to the polypeptide is detected. A test compound which binds to the polypeptide is thereby identified as a potential therapeutic agent for the treatment of malignant neoplasia and more particularly breast cancer.

In even another embodiment the invention provides another composition, method, and kit of screening for agents which regulate the activity of a polypeptide comprising a polypeptide selected from SEQ ID NO: 166 to 330 and 492 to 511. A test compound is contacted with the particular polypeptide. A biological activity mediated by the polypeptide is detected. A test compound which decreases the biological activity is thereby identified as a potential therapeutic agent for decreasing the activity of the particular polypeptide in malignant neoplasia and especially in breast cancer A test compound which increases the biological activity is thereby identified as a potential therapeutic agent for increasing the activity of the particular polypeptide in malignant neoplasia and especially in breast cancer

The invention thus provides polypeptides selected from one of the polypeptides with SEQ ID NO: 166 to 330 and 492 to 511 which can be used to identify compounds which may act, for example, as regulators or modulators such as agonists and antagonists, partial agonists, inverse agonists, activators, co-activators and inhibitors of the polypeptide comprising a polypeptide selected from SEQ ID NO: 166 to 330 and 492 to 511 Accordingly, the invention provides reagents and compositions, methods, and kits for regulating a polypeptide comprising a polypeptide selected from SEQ ID NO: 166 to 330 and 492 to 511 in malignant neoplasia and more particularly breast cancer. The regulation can be an up- or down regulation. Reagents that modulate the expression, stability or amount of a polynucleotide listed in Table 1a and 1b (SEQ ID NO: 1 to 165 and 472 to 491 or the activity of the polypeptide comprising a polypeptide selected from SEQ ID NO: 166 to 330 and 492 to 511 can be a protein, a peptide, a peptidomimetic, a nucleic acid, a nucleic acid analogue (e.g. peptide nucleic acid, locked nucleic acid) or a small molecule. Compositions, methods, and kits that modulate the expression, stability or amount of a polynucleotide comprising a polynucleotide selected from SEQ ID NO: 1 to 165 and 472 to 491 (listed in Table 1a and 1b) or the activity of the polypeptide comprising a polypeptide selected from SEQ ID NO: 166 to 330 and 492 to 511 (Table 1) can be gene replacement therapies, antisense, ribozyme and triplex nucleic acid approaches.

The invention further provides a composition, method, and kit of making an isolated hybridoma which produces an antibody useful for assessing whether a patient is afflicted with breast cancer. The composition, method, and kit comprises isolating a protein encoded by a marker gene listed within Table 1a and 1b or a polypeptide fragment of the protein, immunizing a mammal using the isolated protein or polypeptide fragment, isolating splenocytes from the immunized mammal, fusing the isolated splenocytes with an immortalized cell line to form hybridomas, and screening individual hybridomas for production of an antibody which specifically binds with the protein or polypeptide fragment to isolate the hybridoma. The invention also includes an antibody produced by this method. Such antibodies specifically bind to a full-length or partial polypeptide comprising a polypeptide selected from SEQ ID NO: 166 to 330 and 492 to 511 (listed in Table 1a and 1b) for use in prediction, prevention, diagnosis, prognosis and treatment of malignant neoplasia and breast cancer in particular.

Yet another embodiment of the invention is the use of a reagent which specifically binds to a polynucleotide comprising a polynucleotide selected from SEQ ID NO: 1 to 165 and 472 to 491 or to a polypeptide comprising a polypeptide selected from SEQ ID NO: 166 to 330 and 492 to 511 (listed in Table 1a and 1b) in the preparation of a medicament for the treatment of malignant neoplasia and breast cancer in particular.

Still another embodiment is the use of a reagent that modulates the activity or stability of a polypeptide comprising a polypeptide selected from SEQ ID NO: 166 to 330 and 492 to 511 (Table 1a and 1b) or the expression, amount or stability of a polynucleotide comprising a polynucleotide selected from SEQ ID NO: 1 to 165 and 472 to 491 (Table 1a and 1b) in the preparation of a medicament for the treatment of malignant neoplasia and breast cancer in particular.

Still another embodiment of the invention is a pharmaceutical composition which includes a reagent which specifically binds to a polynucleotide comprising a polynucleotide selected from SEQ ID NO: 1 to 165 (Table 1) or a polypeptide comprising a polypeptide selected from SEQ ID NO: 166 to 300, and a pharmaceutically acceptable carrier.

A further embodiment of the invention is a pharmaceutical composition comprising a polynucleotide including a sequence which hybridizes under stringent conditions to a polynucleotide comprising a polynucleotide selected from SEQ ID NO: 1 to 165 and 472 to 491 and encoding a polypeptide exhibiting the same biological function as given for the respective polynucleotide in Table 1a and 1b or 4, or encoding a polypeptide comprising a polypeptide selected from SEQ ID NO: 166 to 330 and 492 to 511. Pharmaceutical compositions, useful in the present invention may further include fusion proteins comprising a polypeptide comprising a polynucleotide selected from SEQ ID NO: 1 to 165 and 472 to 491, or a fragment thereof, antibodies, or antibody fragments

The invention also provides various kits. Such kit comprises reagents for assessing expression of a single or a plurality of genes selected from the marker genes listed in Table 1a and 1b or selected from the sub-set of genes listed in Table 2.

In one aspect, the invention provides a kit for assessing whether a patient is afflicted with breast cancer.

In another aspect, the invention provides a kit for assessing the suitability of each of a plurality of compounds for inhibiting a breast cancer in a patient. The kit comprises reagents for assessing expression of a marker gene listed within Table 1a and 1b, or reagents for assessing the expression of each marker gene of a marker gene set listed in Table 2. The kit may also comprise a plurality of compounds.

In an additional aspect, the invention provides a kit for assessing the presence of breast cancer cells. This kit comprises an antibody, wherein the antibody binds specifically with a protein encoded by a marker gene listed within Table 1a and 1b or polypeptide fragment of the protein. The kit may also comprise a plurality of antibodies, wherein the plurality binds specifically with-the protein encoded by each marker gene of a marker gene set listed in Table 2.

In yet another aspect, the invention provides a kit for assessing the presence of breast cancer cells, wherein the kit comprises a nucleic acid probe. The probe hybridizes specifically with a RNA transcript of a marker gene listed within Table 1a and 1b or cDNA of the transcript. The kit may also comprise a plurality of probes, wherein each of the probes hybridizes specifically with a RNA transcript of one of the marker genes of a marker gene set listed in Table 2.

It will be appreciated that the compositions, methods, and kits of the present invention may also include known cancer marker genes including known breast cancer marker genes. It will further be appreciated that the compositions, methods, and kits may be used to identify cancers other than breast cancer.

### DETAILED DESCRIPTION OF THE INVENTION

### Definitions

“Differential expression”, or “expression” as used herein, refers to both quantitative as well as qualitative differences in the genes' expression patterns depending on differential development, different genetic background of tumor cells and/or reaction to the tissue environment of the tumor. Differentially expressed genes may represent “marker genes,” and/or “target genes”. The expression pattern of a differentially expressed gene disclosed herein may be utilized as part of a prognostic or diagnostic breast cancer evaluation. Alternatively, a differentially expressed gene disclosed herein may be used in methods for identifying reagents and compounds and uses of these reagents and compounds for the treatment of breast cancer as well as methods of treatment. The differential regulation of the gene is not limited to a specific cancer cell type or clone, but rather displays the interplay of cancer cells, muscle cells, stromal cells, connective tissue cells, other epithelial cells, endothelial cells and blood vessels as well as cells of the immune system (e.g. lymphocytes, macrophages, killer cells).

“Biological activity” or “bioactivity” or “activity” or “biological function”, which are used interchangeably, herein mean an effector or antigenic function that is directly or indirectly performed by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers a biological molecule, e.g., a nucleic acid, peptide, hormone, etc., whose presence or concentration can be detected and correlated with a known condition, such as a disease state.

The term “marker gene,” as used herein, refers to a differentially expressed gene which expression pattern may be utilized as part of predictive, prognostic or diagnostic process in malignant neoplasia or breast cancer evaluation, or which, alternatively, may be used in methods for identifying compounds useful for the treatment or prevention of malignant neoplasia and breast cancer in particular. A marker gene may also have the characteristics of a target gene.

“Target gene”, as used herein, refers to a differentially expressed gene involved in breast cancer in a manner by which modulation of the level of target gene expression or of target gene product activity may act to ameliorate symptoms of malignant neoplasia and breast cancer in particular. A target gene may also have the characteristics of a marker gene.

The term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The term “cancer” is not limited to any stage, grade, histomorphological feature, invasiveness, agressivity or malignancie of an affected tissue or cell aggregation. In particular stage 0 breast cancer, stage I breast cancer, stage II breast cancer, stage III breast cancer, stage IV breast cancer, grade I breast cancer, grade II breast cancer, grade III breast cancer, malignant breast cancer, primary carcinomas of the breast, and all other types of cancers, malignancies and transformations associated with the breast are included. The terms “neoplastic lesion” or “neoplastic disease” or “neoplasia” or “cancer” are not limited to any tissue or cell type they also include primary, secondary or metastatic lesion of cancer patients, and also comprises lymph nodes affected by cancer cells or minimal residual disease cells either locally deposited (e.g. bone marrow, liver, kidney) or freely floating throughout the patients body.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, cell-containing body fluids, free floating nucleic acids, urine, peritoneal fluid, and pleural fluid, or cells therefrom. Biological samples may also include sections of tissues such as frozen or fixed sections taken for histological purposes. A biological sample to be analyzed is tissue material from neoplastic lesion taken by aspiration or punctuation, excision or by any other surgical method leading to biopsy or resected cellular material. Such biological sample may comprises cells obtained from a patient. The cells may be found in a breast cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “therapy modality”, “therapy mode”, “regimen” or “chemo regimen” as well as “therapy regime” refers to a timely sequential or simultaneous administration of anti tumor, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in dose of the single agent, timeframe of application and frequency of administration within a defined therapy window. Currently various combinations of various drugs and/or physical methods, and various schedules are under investigation.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide, a ligand or an enzyme substrate. An antagonist can also be a compound that downregulates expression of a gene or which reduces the amount of expressed protein present.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation (i.e., activation or stimulation (e.g., by agonizing or potentiating) and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “nucleotide analog” refers to oligomers or polymers being at least in one feature different from naturally occurring nucleotides, oligonucleotides or polynucleotides, but exhibiting functional features of the respective naturally occurring nucleotides (e.g. base paring, hybridization, coding information) and that can be used for said compositions. The nucleotide analogs can consist of non-naturally occurring bases or polymer backbones, examples of which are LNAs, PNAs and Morpholinos. The nucleotide analog has at least one molecule different from its naturally occurring counterpart or equivalent.

“BREAST CANCER GENES” or “BREAST CANCER GENE” as used herein refers to the polynucleotides of SEQ ID NO:1 to 165 and 472 to 491 (listed in Table 1a and 1b), as well as derivatives, fragments, analogs and homologues thereof, the polypeptides encoded thereby, (SEQ ID NO:166 to 330 and 492 to 511, see Table 1) as well as derivatives, fragments, analogs and homologues thereof and the corresponding genomic transcription units which can be derived or identified with standard techniques well known in the art using the information disclosed in Tables 1 to 5. The Genename, Reference Sequence, unique Gene-identifier, and the Locuslink ID numbers of the polynucleotide sequences of the SEQ ID NO: 1 to 65 and the polypeptides of the SEQ ID NO: 166 to 330 and 492 to 511 are shown in Table 1a and 1b, the gene description, gene function and subcellelar localization is given in Tables 4a and 4b.

The term “chromosomal region” as used herein refers to a consecutive DNA stretch on a chromosome which can be defined by cytogenetic or other genetic markers such as e.g. restriction length polymorphisms (RFLPs), single nucleotide polymorphisms (SNPs), expressed sequence tags (ESTs), sequence tagged sites (STSs), microsatellites, variable number of tandem repeats (VNTRs) and genes. Typically a chromosomal region consists of up to 2 Megabases (MB), up to 4 MB, up to 6 MB, up to 8 MB, up to 10 MB, up to 20 MB or even more MB.

The term “kit” as used herein refers to any manufacture (e.g. a diagnostic or research product) comprising at least one reagent, e.g. a probe, for specifically detecting the expression of at least one marker gene disclosed in the invention, in particular of those genes listed in Table 2, whereas the manufacture is being sold, distributed, and/or promoted as a unit for performing the methods of the present invention. The genes, primer and probes listed in Table 2 and 5 or any combination of at least two of them, regard as one single test for the purposes, methods and disclosures of this invention. Also reagents (e.g. immunoassays) to detect the presence, the stability, activity, complexity of the respective marker gene products comprising polypeptides selected from SEQ ID NO:166 to 330 and 492 to 511 regard as components of the kit. In addition, any combination of nucleic acid and protein detection as disclosed in the invention are regard as a kit.

The present invention provides polynucleotide sequences and proteins encoded thereby, as well as probes derived from the polynucleotide sequences, antibodies directed to the encoded proteins, and predictive, preventive, diagnostic, prognostic and therapeutic uses for individuals which are at risk for or which have malignant neoplasia and breast cancer in particular. The sequences disclosure herein have been found to be differentially expressed in samples from breast cancer.

The present invention is based on the identification of 185 genes that are differentially regulated (up- or down regulated) in tumor biopsies of patients with clinical evidence of breast cancer. The characterization of the co-expression of some of these genes provides newly identified roles in breast cancer. The gene names, the database accession numbers (Genename, Reference Sequence, unique Gene-identifier, and the Locuslink ID numbers) as well as the putative or known functions of the encoded proteins and their subcellular localization are given in Tables 1 to 4a and 4b. The primer sequences used for the gene amplification and hybridization probes are shown in Table 5.

The present invention relates to:
1. A method for characterizing (preferably ex vivo) the state of a neoplastic disease in a subject, comprising
   (i) determining the pattern of expression levels of at least 6, 8, 10, 15, 20, 30, or 47 marker genes, comprised in a group of marker genes consisting of SEQ ID NO: 1 to 165 and 472 to 491, in a biological sample from said subject,
   (ii) comparing the pattern of expression levels determined in (i) with one or several reference pattern(s) of expression levels,
   (iii) characterizing the state of said neoplastic disease in said subject from the outcome of the comparison in step (ii).
2. A method for detection, diagnosis, screening, monitoring, and/or prognosis of a neoplastic disease in a subject, (preferably ex vivo) comprising
   (i) determining the pattern of expression levels of at least 1, 2, 3, 5, 10, 15, 20, 30, or 47 marker genes, comprised in a group of marker genes consisting of SEQ ID NOs:1 to 17, 19 to 33, 35 to 50, 52 to 64, 66 to 85, 88 to 91, and 93 to 165 and 472 to 491 in biological samples from said subject,
   (ii) comparing the pattern of expression levels determined in (i) with one or several reference pattern(s) of expression levels,
   (iii) detecting, diagnosing, screening, monitoring, and/or prognosing said neoplastic disease in said subject from the outcome of the comparison in step (ii).

Determination of an expression level can comprise a quantitatification of the expression level and/or a purely qualitative determination of the expression level.

A “pattern of expression levels” of a single gene is to be understood as the expression level of said gene as determined by suitable methods.

Nucleic acid molecules, referred to with a specific SEQ ID NO, within the meaning of the invention, are to be understood as comprising also variants of said nucleic acid molecules, which can be derived from the original nucleic acid molecules by deletion, insertion or transposition of nucleotides, provided said variants still have an 80, 90, 95, or 99% sequence identity towards the original sequence. Preferrably the variants still have the same biological activity and/or function as have the original molecules.

It is obvious to the person skilled in the art that a reference to a nucleotide sequence is meant to comprise the reference to the associated protein sequence which is coded by said nucleotide sequence.

“% identity” of a first sequence towards a second sequence, within the meaning of the invention, means the % identity which is calculated as follows: First the optimal global alignment between the two sequences is determined with the CLUSTALW algorithm [Thomson J D, Higgins D G, Gibson T J. 1994. ClustalW: Improving the sensitivity of progressive multiple sequence alignment through sequence weighting, positions-specific gap penalties and weight matrix choice. Nucleic Acids Res., 22: 4673-4680], Version 1.8, applying the following command line syntax: ./clustalw-infile=./infile.txt-output=outorder=aligned-pwmatrix=gonnet-pwdnamatrix=clustalw-pwgapopen=10.0-pwgapext=0.1-matrix=gonnet-gapopen=10.0-gapext-0.05-gapdist=8-hgapresidues=GPSNDQERK-maxdiv=40. Implementations of the CLUSTAL W algorithm are readily available at numerous sites on the internet, including, e.g., http://www.ebi.ac.uk. Thereafter, the number of matches in the alignment is determined by counting the number of identical nucleotides (or amino acid residues) in aligned positions. Finally, the total number of matches is divided by the number of nucleotides (or amino acid residues) of the longer of the two sequences, and multiplied by 100 to yield the % identity of the first sequence towards the second sequence.
3. A method of count 1 or 2, wherein said method comprises multiple determinations of a pattern of expression levels, at different points in time, thereby allowing to monitor the development of said neoplastic disease in said subject.
4. A method of count 1, wherein said method comprises an estimation of the likelihood of success of a given mode of treatment for said neoplastic disease in said subject.
5. A method of count 1, wherein said method comprises an assessment of whether the subject is expected to respond or whether the subject is expected not to a given mode of treatment for said neoplastic disease.

The terms “to respond” or “not to respond” are to be understood in a qualitative and/or in a quantitative fashion. “To respond” and “not to respond” is to be assessed with regard to a suitable reference responses, such as, e.g., responses shown by “responders” and “not-responders” to a certain mode of treatment or modality of treatment.
6. A method of count 4 or 5, wherein a predictive algorithm is used.

Predictive algorithms, which are well known to a person skilled in the art of data analysis, are to be understood as being any kind of predictive algorithm known in the art. Preferred examples of such algorithms are, e.g., the SVM algorithm disclosed in Example 4.
7. A method of count 6, wherein the predictive algorithm is a Support Vector Machine.

Support Vector Machines are algorithms, well known to the person skilled in the art of data analysis. A Support Vector Machine algorithm is disclosed in Example 4.
8. A method of any of counts 4 to 7, wherein said given mode of treatment
   (i) acts on cell proliferation, and/or
   (ii) acts on cell survival, and/or
   (iii) acts on cell motility, and/or
   (iv) is an anthracycline based mode of treatment, and/or
   (v) comprises administration of epirubicin and/or cyclophoshamid.
9. A method of treatment for a subject afflicted with a neoplastic disease, comprising
   (i) identifying a promising mode of treatment with the method of count 4 or 5,
   (ii) treating said neoplastic disease in said patient by the mode of treatment identified in step (i).
10. A method of screening for subjects afflicted with a neoplastic disease, wherein the method of count 1 or 2 is applied to a plurality of subjects.
11. A method of screening for substances and/or therapy modalities having curative effect on a neoplastic disease comprising
   (i) obtaining a biological sample from a subject afflicted with said neoplastic disease,
   (ii) assessing, from said biological sample, using the method of count 4 or 5, whether said subject is expected to respond to a given mode of treatment for said neoplastic disease,
   (iii) if said subject is expected to respond to said given mode of treatment, incubating said biological sample with said substance under said therapy modalities,
   (iv) observing changes in said biological sample triggered by said test substance under said therapy modalities,
   (v) selecting or rejecting said test substance and/or said therapy modalities, based on the observation of changes in said biological sample under (iv).

Selecting specific biological samples of, e.g., good responders to a given therapy can help to identify novel substances and/or therapy modalities for the treatment of said specific neoplastic disease.
12. A method of screening for compounds having curative effect on a neoplastic disease comprising
   (i) incubating biological samples or extracts of these with a test substance,
   (ii) determining the pattern of expression levels of at least 1, 2, 3, 5, 10, 15, 20, 30, or 47 marker genes, comprised in a group of marker genes consisting of SEQ ID NO:1 to 17, 19 to 33, 35 to 50, 52 to 64, 66 to 85, 88 to 91, and 93 to 165 and 472 to 491 in said biological sample,
   (iii) comparing the pattern of expression levels determined in (ii) with one or several reference pattern(s),
   (iv) selecting or rejecting said test substance, based on the comparison performed under (iii).
13. A method of any of counts 1 to 12 wherein said marker genes are comprised in a group of marker genes listed in Table 2.

Marker genes listed in Table 2 are shown to be particularly informative with respect to assessing the probability of success of a certain mode of treatment for a given neoplastic disease. Marker genes of Table 2 are preferred marker genes, according to the invention.
14. A method of any of counts 1 to 13, wherein the expression level is determined
   (i) with a hybridization based method, or
   (ii) with a hybridization based method utilizing arrayed probes, or
   (iii) with a hybridization based method utilizing individually labeled probes, or
   (iv) by real time real time PCR, or
   (v) by assessing the expression of polypeptides, proteins or derivatives thereof, or
   (vi) by assessing the amount of polypeptides, proteins or derivatives thereof.
15. A method of any of counts 1 to 14, wherein the neoplastic disease is breast cancer.

The methods of the invention are preferably performed ex vivo. More preferably, methods of the invention are performed ex vivo on samples that are already available or can be obtained without intervention of a physician or other medically trained personnel.
16. A kit comprising at least 6, 8, 10, 15, 20, 30, or 47 primer pairs and probes suitable for marker genes comprised in a group of marker genes consisting of
   (i) SEQ ID NO:1 to SEQ ID NO:165, or
   (ii) the marker genes listed in Table 2.
17. A kit comprising at least 6, 8, 10, 15, 20, 30, or 47 sets of individually labeled probes, each having a sequence comprised in a group of sequences consisting of SEQ ID NO:331 to SEQ ID NO:471.
18. A kit comprising at least 6, 8, 10, 15, 20, 30, or 47 sets of arrayed probes, each having a sequence comprised in a group of sequences consisting of SEQ ID NO:331 to SEQ ID NO:471.
   Biological Relevance of the Genes which are Part of the Invention

Some of the genes listed in Table 1a and 1b represent biological, cellular processes and are characterized by similar regulation of genes. By the way of illustration but limited to the following examples a few characteristic genes from Table 1 are described in later by greater detail:

### MAD2L1

The initiation of chromosome segregation at anaphase is linked by the spindle assembly checkpoint to the completion of chromosome-microtubule attachment during metaphase. To determine the function of the Mad2 protein during normal cell division, knock out experiments in mice were performed. These cells were unable to arrest in response to spindle disruption. At embryonic day 6.5, the cells of the epiblast began rapid cell division, and the absence of a checkpoint resulted in widespread chromosome missegregation and apoptosis. In contrast, the postmitotic trophoblast giant cells survived without Mad2. Thus, the spindle assembly checkpoint is required for accurate chromosome segregation in mitotic mouse cells and for embryonic viability, even in the absence of spindle damage.

Meiosis I nondisjunction in spindle checkpoint mutants could be prevented by delaying the onset of anaphase. In a recombinant-defective mutant, the checkpoint delayed the biochemical events of anaphase I, suggesting that chromosomes that are attached to microtubules but are not under tension can activate the spindle checkpoint. Spindle checkpoint mutants reduced the accuracy of chromosome segregation in meiosis I much more than that in meiosis II, suggesting that checkpoint defects may contribute to Down syndrome and possibly to the “chaotic” polyploidy observed in cancer.

### IGFBP4

Seven structurally distinct insulin-like growth factor binding proteins have been isolated and their cDNAs cloned: IGFBP1, IGFBP2, IGFBP3, IGFBP4, IGFBP5, IGFBP6, and IGFBP7. The proteins display strong sequence homologies, suggesting that they are encoded by a closely related family of genes. The IGFBPs contain 3 structurally distinct domains each comprising approximately one-third of the molecule. The N-terminal domain 1 and the C-terminal domain 3 of the 6 human IGFBPs show moderate to high levels of sequence identity including 12 and 6 invariant cysteine residues in domains 1 and 3, respectively (IGFBP6 contains 10 cysteine residues in domain 1), and are thought to be the IGF binding domains. Domain 2 is defined primarily by a lack of sequence identity among the 6 IGFBPs and by a lack of cysteine residues, though it does contain 2 cysteines in IGFBP4. Domain 3 is homologous to the thyroglobulin type I repeat unit. Studies suggested that the primary effect of the proteins is the attenuation of IGF activity and suggested that they contribute to the control of IGF-mediated cell growth and metabolism

### DDB2

In human cells, efficient global genomic repair of DNA damage induced by ultraviolet radiation requires the p53 tumor suppressor. The p48 gene is required for expression of an ultraviolet radiation-damaged DNA-binding activity and is disrupted by mutations in the subset of xeroderma pigmentosum group E cells that lack this activity, DDB-negative XPE. p48 mRNA levels are strongly depend on basal p53 expression and increase further after DNA damage in a p53-dependent manner. Furthermore, like p53−/−cells, xeroderma pigmentosum group E cells are deficient in global genomic repair. These results identified p48 as a link between p53 and the nucleotide excision-repair apparatus.

UV-damaged DNA-binding activity (UV-DDB) is deficient in cell lines and primary tissues from rodents. Transfection of p48 conferred UV-DDB to hamster cells and enhanced removal of cyclobutane pyrimidine dimers (CPDs) from genomic DNA and from the nontranscribed strand of an expressed gene. Expression of p48 suppressed UV-induced mutations arising from the nontranscribed strand but had no effect on cellular UV sensitivity. The results defined the role of p48 in DNA repair, demonstrated the importance of CPDs in mutagenesis, and suggested how rodent models can be improved to better reflect cancer susceptibility in humans.

### HSPA2

Several heat-shock protein genes are located in the major histocompatibility complex on chromosome 6, e.g., HSPA1. However HSPA2 is located on 14q22-q24. isolated The clone for HSPA2 is characterized by a single open reading frame of 1,917 basepairs that encodes a 639-amino acid protein with a predicted molecular weight of 70,030 Da. Analysis of the sequence indicated that HSPA2 is the human homolog of the murine Hsp70-2 gene with 91.7% identity in the nucleotide coding sequence and 98.2% in the corresponding amino acid sequence. HSPA2 has less amino acid homology to the other members of the human HSP70 gene family. HSPA2 is constitutively expressed in most tissues, with very high levels in testis and skeletal muscle. HSPA2 is expressed abundantly in muscle, heart, esophagus, and brain, and to a lesser extent in testis. A female homozygous knockout mice for Hsp70-2 undergo normal meiosis and is fertile. In contrast, homozygous male knockout mice lacked postmeiotic spermatids and mature sperm and were infertile. Hsp70-2 is normally associated with synaptonemal complexes in the nuclei of meiotic spermatocytes. In the male knockouts, these structures were abnormal by late prophase. One can observe also a large increase in spermatocyte apoptosis.

### Polynucleotides

A “BREAST CANCER GENE” polynucleotide can be single- or double-stranded and comprises a coding sequence or the complement of a coding sequence for a “BREAST CANCER GENE” polypeptide. Degenerate nucleotide sequences encoding human “BREAST CANCER GENE” polypeptides, as well as homologous nucleotide sequences which are at least about 50, 55, 60, 65, 70, preferably about 75, 90, 96, or 98% identical to the nucleotide sequences of SEQ ID NO: 1 to 165 and 472 to 491 also are “BREAST CANCER GENE” polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologues, and variants of “BREAST CANCER GENE” polynucleotides which encode biologically active “BREAST CANCER GENE” polypeptides also are “BREAST CANCER GENE” polynucleotides.

### Preparation of Polynucleotides

A naturally occurring “BREAST CANCER GENE” polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated “BREAST CANCER GENE” polynucleotides. For example, restriction enzymes and probes can be used to isolate polynucleotide fragments which comprises “BREAST CANCER GENE” nucleotide sequences. Isolated polynucleotides are in preparations which are free or at least 70, 80, or 90% free of other molecules.

“BREAST CANCER GENE” cDNA molecules can be made with standard molecular biology techniques, using “BREAST CANCER GENE” mRNA as a template. Any RNA isolation technique which does not select against the isolation of mRNA may be utilized for the purification of such RNA samples. See, for example, Sambrook et al., 1989, (6); and Ausubel, F. M. et al., 1989, (7), both of which are incorporated herein by reference in their entirety. Additionally, large numbers of tissue samples may readily be processed using techniques well known to those of skill in the art, such as, for example, the single-step RNA isolation process of Chomczynski, P. (1989, U.S. Pat. No. 4,843,155), which is incorporated herein by reference in its entirety.

“BREAST CANCER GENE” cDNA molecules can thereafter be replicated using molecular biology techniques known in the art and disclosed in manuals such as Sambrook et al., 1989, (6). An amplification technique, such as PCR, can be used to obtain additional copies of polynucleotides of the invention, using either human genomic DNA or cDNA as a template.

Alternatively, synthetic chemistry techniques can be used to synthesizes “BREAST CANCER GENE” polynucleotides. The degeneracy of the genetic code allows alternate nucleotide sequences to be synthesized which will encode a “BREAST CANCER GENE” polypeptide or a biologically active variant thereof.

### Identification of Differential Expression

Transcripts within the collected RNA samples which represent RNA produced by differentially expressed genes may be identified by utilizing a variety of methods which are ell known to those of skill in the art. For example, differential screening [Tedder, T. F. et al., 1988, (8)], subtractive hybridization [Hedrick, S. M. et al., 1984, (9); Lee, S. W. et al., 1984, (10)], and, preferably, differential display (Liang, P., and Pardee, A. B., 1993, U.S. Pat. No. 5,262,311, which is incorporated herein by reference in its entirety), may be utilized to identify polynucleotide sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the isolated mRNA, and the removal of all hybridized, and therefore double-stranded, sequences. The remaining non-hybridized, single-stranded cDNAs, potentially represent clones derived from genes that are differentially expressed in the two mRNA sources. Such single-stranded cDNAs are then used as the starting material for the construction of a library comprising clones derived from differentially expressed genes.

The differential display technique describes a procedure, utilizing the well known polymerase chain reaction (PCR; the experimental embodiment set forth in Mullis, K. B., 1987, U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times. The pattern of clones resulting from the reverse transcription and amplification of the mRNA of two different cell types is displayed via sequencing gel electrophoresis and compared. Differences in the two banding patterns indicate potentially differentially expressed genes.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ nontranscribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer; ABI), and the entire process from loading of samples to computer analysis and electronic data display can be computer controlled. Capillary electrophoresis is especially preferable for the sequencing of small pieces of DNA which might be present in limited amounts in a particular sample.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR. Upon corroboration, the differentially expressed genes may be further characterized, and may be identified as target and/or marker genes, as discussed, below.

Also, amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

An analysis of the tissue distribution of the mRNA produced by the identified genes may be conducted, utilizing standard techniques well known to those of skill in the art. Such techniques may include, for example, Northern analyses and RT-PCR. Such analyses provide information as to whether the identified genes are expressed in tissues expected to contribute to breast cancer. Such analyses may also provide quantitative information regarding steady state mRNA regulation, yielding data concerning which of the identified genes exhibits a high level of regulation in, preferably, tissues which may be expected to contribute to breast cancer.

Such analyses may also be performed on an isolated cell population of a particular cell type derived from a given tissue. Additionally, standard in situ hybridization techniques may be utilized to provide information regarding which cells within a given tissue express the identified gene. Such analyses may provide information regarding the biological function of an identified gene relative to breast cancer in instances wherein only a subset of the cells within the tissue is thought to be relevant to breast cancer.

### Extending Polynucleotides

In one embodiment of such a procedure for the identification and cloning of full length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA hybrid may then be “tailed” with guanines using a standard terminal transferase reaction, the hybrid may be digested with RNase H, and second strand synthesis may then be primed with a poly-C primer. Using the two primers, the 5′ portion of the gene is amplified using PCR. Sequences obtained may then be isolated and recombined with previously isolated sequences to generate a full-length cDNA of the differentially expressed genes of the invention. For a review of cloning strategies and recombinant DNA techniques, see e.g., Sambrook et al., (6); and Ausubel et al., (7).

Various PCR-based methods can be used to extend the polynucleotide sequences disclosed herein to detect upstream sequences such as promoters and regulatory elements. For example, restriction site PCR uses universal primers to retrieve unknown sequence adjacent to a known locus [Sarkar, 1993, (11)]. Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region [Triglia et al., 1988, (12)]. Primers can be designed using commercially available software, such as OLIGO 4.06 Primer Analysis software (National Biosciences Inc., Plymouth, Minn.), to be e.g. 2230 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA [Lagerstrom et al., 1991, (13))]. In this method, multiple restriction enzyme digestions and ligations also can be used to place an engineered double-stranded sequence into an unknown fragment of the DNA molecule before performing PCR.

Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

The sequences of the identified genes may be used, utilizing standard techniques, to place the genes onto genetic maps, e.g., mouse [Copeland & Jenkins, 1991, (14)] and human genetic maps [Cohen, et al., 1993, (15)]. Such mapping information may yield information regarding the genes' importance to human disease by, for example, identifying genes which map near genetic regions to which known genetic breast cancer tendencies map.

### Identification of Polynucleotide Variants and Homologues or Splice Variants

Variants and homologues of the “BREAST CANCER GENE” polynucleotides described above also are “BREAST CANCER GENE” polynucleotides. Typically, homologous “BREAST CANCER GENE” polynucleotide sequences can be identified by hybridization of candidate polynucleotides to known “BREAST CANCER GENE” polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50 EC once, 30 minutes; then 2×SSC, room temperature twice, 10 minutes each homologous sequences can be identified which contain at most about 25-30% basepair mismatches. More preferably, homologous polynucleotide strands contain 15-25% basepair mismatches, even more preferably 5-15% basepair mismatches.

Species homologues of the “BREAST CANCER GENE” polynucleotides disclosed herein also can be identified by making suitable probes or primers and screening cDNA expression libraries from other species, such as mice, monkeys, or yeast. Human variants of “BREAST CANCER GENE” polynucleotides can be identified, for example, by screening human cDNA expression libraries. It is well known that the Tₘ of a double-stranded DNA decreases by 1-1.5° C. with every 1% decrease in homology [Bonner et al., 1973, (16)]. Variants of human “BREAST CANCER GENE” polynucleotides or “BREAST CANCER GENE” polynucleotides of other species can therefore be identified by hybridizing a putative homologous “BREAST CANCER GENE” polynucleotide with a polynucleotide having a nucleotide sequence of one of the sequences of the SEQ ID NO: 1 to 165 and 472 to 491 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to “BREAST CANCER GENE” polynucleotides or their complements following stringent hybridization and/or wash conditions also are “BREAST CANCER GENE” polynucleotides. Stringent wash conditions are well known and understood in the art and are disclosed, for example, in Sambrook et al., (6). Typically, for stringent hybridization conditions a combination of temperature and salt concentration should be chosen that is approximately 12 to 20° C. below the calculated Tₘ of the hybrid under study. The Tₘ of a hybrid between a “BREAST CANCER GENE” polynucleotide having a nucleotide sequence of one of the sequences of the SEQ ID NO: 1 to 165 and 472 to 491 or the complement thereof and a polynucleotide sequence which is at least about 50, preferably about 75, 90, 96, or 98% identical to one of those nucleotide sequences can be calculated, for example, using the equation below [Bolton and McCarthy, 1962, (17):

Tₘ=81.5° C.−16.6(log₁₀[Na⁺])+0.41(% G+C)−0.63(% formamide)−600/l),
where l=the length of the hybrid in basepairs.

Stringent wash conditions include, for example, 4×SSC at 65° C., or 50% formamide, 4×SSC at 28° C., or 0.5×SSC, 0.1% SDS at 65° C. Highly stringent wash conditions include, for example, 0.2×SSC at 65° C.

The biological function of the identified genes may be more directly assessed by utilizing relevant in vivo and in vitro systems. In vivo systems may include, but are not limited to, animal systems which naturally exhibit breast cancer predisposition, or ones which have been engineered to exhibit such symptoms, including but not limited to oncogene overexpression (e.g. HER2/neu, ras, raf, or EGFR) malignant neoplasia mouse.

Splice variants derived from the same genomic region, encoded by the same pre mRNA can be identified by hybridization conditions described above for homology search. The specific characteristics of variant proteins encoded by splice variants of the same pre transcript may differ and can also be assayed as disclosed. A “BREAST CANCER GENE” polynucleotide having a nucleotide sequence of one of the sequences of the SEQ ID NO: 1 to 165 and 472 to 491 or the complement thereof may therefor differ in parts of the entire sequence. The prediction of splicing events and the identification of the utilized acceptor and donor sites within the pre mRNA can be computed (e.g. Software Package GRAIL or GenomeSCAN) and verified by PCR method by those with skill in the art.

### Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 6 nucleotides in length, but can be at least 7, 8, 10, 12, 15, 20, 25, 30, 35, 40, 45, or 50 or more nucleotides long. Longer sequences also can be used. Antisense oligonucleotide molecules can be provided in a DNA construct and introduced into a cell as described above to alter the level of “BREAST CANCER GENE” gene products in the cell.

Antisense oligonucleotides can be deoxyribonucleotides, ribonucleotides, peptide nucleic acids (PNAs; described in U.S. Pat. No. 5,714,331), locked nucleic acids (LNAs; described in WO 99/12826), or a combination of them. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters [Brown, 1994, (55); Sonveaux, 1994, (56) and Uhlmann et al., 1990, (57)].

Modifications of “BREAST CANCER GENE” expression can be obtained by designing antisense oligonucleotides which will form duplexes to the control, 5′, or regulatory regions of the “BREAST CANCER GENE”. Oligonucleotides derived from the transcription initiation site, e.g., between positions 10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature [Gee et al., 1994, (58)]. An antisense oligonucleotide also can be designed to block translation of mRNA by preventing the transcript from binding to ribosomes.

Precise complementarity is not required for successful complex formation between an antisense oligonucleotide and the complementary sequence of a “BREAST CANCER GENE” polynucleotide. Antisense oligonucleotides which comprise, for example, 2, 3, 4, or 5 or more stretches of contiguous nucleotides which are precisely complementary to a “BREAST CANCER GENE” polynucleotide, each separated by a stretch of contiguous nucleotides which are not complementary to adjacent “BREAST CANCER GENE” nucleotides, can provide sufficient targeting specificity for “BREAST CANCER GENE” mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular “BREAST CANCER GENE” polynucleotide sequence.

Antisense oligonucleotides can be modified without affecting their ability to hybridize to a “BREAST CANCER GENE” polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′ substituted oligonucleotide in which the 3′ hydroxyl group or the 5′ phosphate group are substituted, also can be employed in a modified antisense oligonucleotide. These modified oligonucleotides can be prepared by methods well known in the art [Agrawal et al., 1992, (59); Uhlmann et al., 1987, (57) and Uhlmann et al., 2000 (60)].

### Ribozymes

Ribozymes are RNA molecules with catalytic activity [Cech, 1987, (61); Cech, 1990, (62) and Couture & Stinchcomb, 1996, (63)]. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The transcribed sequence of a “BREAST CANCER GENE” can be used to generate ribozymes which will specifically bind to mRNA transcribed from a “BREAST CANCER GENE” genomic locus. Methods of designing and constructing ribozymes which can cleave other RNA molecules in trans in a highly sequence specific manner have been developed and described in the art [Haseloff et al., 1988, (64)]. For example, the cleavage activity of ribozymes can be targeted to specific RNAs by engineering a discrete “hybridization” region into the ribozyme. The hybridization region contains a sequence complementary to the target RNA and thus specifically hybridizes with the target [see, for example, Gerlach et al., EP 0 321201].

Specific ribozyme cleavage sites within a “BREAST CANCER GENE” RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate “BREAST CANCER GENE” RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease “BREAST CANCER GENE” expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

### Polypeptides

“BREAST CANCER GENE” polypeptides according to the invention comprise an polypeptide selected from SEQ ID NO: 166 to 330 and 492 to 511 or encoded by any of the polynucleotide sequences of the SEQ ID NO: 1 to 165 and 472 to 491 or derivatives, fragments, analogues and homologues thereof. A BREAST CANCER GENE” polypeptide of the invention therefore can be a portion, a full-length, or a fusion protein comprising all or a portion of a “BREAST CANCER GENE” polypeptide.

### Protein Purification

“BREAST CANCER GENE” polypeptides can be purified from any cell which expresses the responding protein, including host cells which have been transfected with “BREAST CANCER GENE” expression constructs. A purified “BREAST CANCER GENE” polypeptide is separated from other compounds which are normally associate with the “BREAST CANCER GENE” polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified ,“BREAST CANCER GENE” polypeptides is at least 80% pure; preferably, the preparations are 90%, 95%, or 99% pure. Purity of the preparations can be assessed by any means known in the art, such as SDS-polyacrylamide gel electrophoresis.

### Obtaining Polypeptides

“BREAST CANCER GENE” polypeptides can be obtained, for example, by purification from human cells, by expression of “BREAST CANCER GENE” polynucleotides, or by direct chemical synthesis.

### Biologically Active Variants

“BREAST CANCER GENE” polypeptide variants which are biologically active, i.e., retain an “BREAST CANCER GENE” activity, can be also regarded as “BREAST CANCER GENE” polypeptides. Preferably, naturally or non-naturally occurring “BREAST CANCER GENE” polypeptide variants have amino acid sequences which are at least about 60, 65, or 70, preferably about 75, 80, 85, 90, 92, 94, 96, or 98% identical to any of the amino acid sequences of the polypeptides of SEQ ID NO: 166 to 330 and 492 to 511 or the polypeptides encoded by any of the polynucleotides of SEQ ID NO: 1 to 165 and 472 to 491 or a fragment thereof. Percent identity between a putative “BREAST CANCER GENE” polypeptide variant and of the polypeptides of SEQ ID NO: 166 to 330 and 492 to 511 polypeptides encoded by any of the polynucleotides of SEQ ID NO: 1 to 165 and 472 to 491 or a fragment thereof is determined by conventional methods. [See, for example, Altschul et al., 1986, (19) and Henikoff & Henikoff, 1992, (20)]. Briefly, two amino acid sequences are aligned to optimize the alignment scores using a gap opening penalty of 10, a gap extension penalty of 1, and the “BLOSUM62” scoring matrix of Henikoff & Henikoff, 1992 (20).

Those skilled in the art appreciate that there are many established algorithms available to align two amino acid sequences. The “FASTA” similarity search algorithm of Pearson & Lipman is a suitable protein alignment method for examining the level of identity shared by an amino acid sequence disclosed herein and the amino acid sequence of a putative variant [Pearson & Lipman, 1988, (21), and Pearson, 1990, (22)]. Briefly, FASTA first characterizes sequence similarity by identifying regions shared by the query sequence (e.g., SEQ ID NO: 1 to 165 and 472 to 491) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm [Needleman & Wunsch, 1970, (23), and Sellers, 1974, (24)], which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, (22).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a “BREAST CANCER GENE” polypeptide can be found using computer programs well known in the art, such as DNASTAR software. Whether an amino acid change results in a biologically active “BREAST CANCER GENE” polypeptide can readily be determined by assaying for “BREAST CANCER GENE” activity, as described for example, in the specific Examples, below. Larger insertions or deletions can also be caused by alternative splicing. Protein domains can be inserted or deleted without altering the main activity of the protein.

### Fusion Proteins

Fusion proteins are useful for generating antibodies against “BREAST CANCER GENE” polypeptide amino acid sequences and for use in various assay systems. For example, fusion proteins can be used to identify proteins which interact with portions of a “BREAST CANCER GENE” polypeptide. Protein affinity chromatography or library-based assays for protein-protein interactions, such as the yeast two-hybrid or phage display systems, can be used for this purpose. Such methods are well known in the art and also can be used as drug screens.

A “BREAST CANCER GENE” polypeptide fusion protein comprises two polypeptide segments fused together by means of a peptide bond. The first polypeptide segment comprises at least 25, 50, 75, 100, 150, 200, 300, 400, 500, 600, 700 or 750 contiguous amino acids of an amino acid sequence encoded by any polynucleotide sequences of the SEQ ID NO: 1 to 165 and 472 to 491 or of a biologically active variant, such as those described above. The first polypeptide segment also can comprise full-length “BREAST CANCER GENE”.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-5-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the “BREAST CANCER GENE” polypeptide-encoding sequence and the heterologous protein sequence, so that the “BREAST CANCER GENE” polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from any of the polynucleotide sequences of the SEQ ID NO: 1 to 165 and 472 to 491 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

### Identification of Species Homologues

Species homologues of human a “BREAST CANCER GENE” polypeptide can be obtained using “BREAST CANCER GENE” polynucleotides (described below) to make suitable probes or primers for screening cDNA expression libraries from other species, such as mice, monkeys, or yeast, identifying cDNAs which encode homologues of a “BREAST CANCER GENE” polypeptide, and expressing the cDNAs as is known in the art.

### Expression of Polynucleotides

To express a “BREAST CANCER GENE” polynucleotide, the polynucleotide can be inserted into an expression vector which contains the necessary elements for the transcription and translation of the inserted coding sequence. Methods which are well known to those skilled in the art can be used to construct expression vectors containing sequences encoding “BREAST CANCER GENE” polypeptides and appropriate transcriptional and translational control elements. These methods include in vitro recombinant DNA techniques, synthetic techniques, and in vivo genetic recombination. Such techniques are described, for example, in Sambrook et al., (6) and in Ausubel et al., (7).

A variety of expression vector/host systems can be utilized to contain and express sequences encoding a “BREAST CANCER GENE” polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those regions of the vector enhancers, promoters, 5′ and 3′ untranslated regions which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a “BREAST CANCER GENE” polypeptide, vectors based on SV40 or EBV can be used with an appropriate selectable marker.

### Bacterial and Yeast Expression Systems

In bacterial systems, a number of expression vectors can be selected depending upon the use intended for the “BREAST CANCER GENE” polypeptide. For example, when a large quantity of the “BREAST CANCER GENE” polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the “BREAST CANCER GENE” polypeptide can be ligated into the vector in frame with sequences for the amino terminal Met and the subsequent 7 residues of β-galactosidase so that a hybrid protein is produced. pIN vectors [Van Heeke & Schuster, (113)] or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al., (7) and Grant et al., (114).

### Plant and Insect Expression Systems

If plant expression vectors are used, the expression of sequences encoding “BREAST CANCER GENE” polypeptides can be driven by any of a number of promoters. For example, viral promoters such as the 35S and 19S promoters of CaMV can be used alone or in combination with the omega leader sequence from TMV [Takamatsu, 1987, (25)]. Alternatively, plant promoters such as the small subunit of RUBISCO or heat shock promoters can be used [Coruzzi et al., 1984, (26); Broglie et al., 1984, (27); Winter et al., 1991, (28)]. These constructs can be introduced into plant cells by direct DNA transformation or by pathogen-mediated transfection. Such techniques are described in a number of generally available reviews.

An insect system also can be used to express a “BREAST CANCER GENE” polypeptide. For example, in one such system Autographa californica nuclear polyhedrosis virus (AcNPV) is used as a vector to express foreign genes in Spodoptera frugiperda cells or in Trichoplusia larvae. Sequences encoding “BREAST CANCER GENE” polypeptides can be cloned into a nonessential region of the virus, such as the polyhedrin gene, and placed under control of the polyhedrin promoter. Successful insertion of “BREAST CANCER GENE” polypeptides will render the polyhedrin gene inactive and produce recombinant virus lacking coat protein. The recombinant viruses can then be used to infect S. frugiperda cells or Trichoplusia larvae in which “BREAST CANCER GENE” polypeptides can be expressed [Engelhard et al., 1994, (29)].

### Mammalian Expression Systems

A number of viral-based expression systems can be used to express “BREAST CANCER GENE” polypeptides in mammalian host cells. For example, if an adenovirus is used as an expression vector, sequences encoding “BREAST CANCER GENE” polypeptides can be ligated into an adenovirus transcription/translation complex comprising the late promoter and tripartite leader sequence. Insertion in a nonessential E1 or E3 region of the viral genome can be used to obtain a viable virus which is capable of expressing a “BREAST CANCER GENE” polypeptide in infected host cells [Logan & Shenk, 1984, (30)]. If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding “BREAST CANCER GENE” polypeptides. Such signals include the ATG initiation codon and adjacent sequences. In cases where sequences encoding a “BREAST CANCER GENE” polypeptide, its initiation codon, and upstream sequences are inserted into the appropriate expression vector, no additional transcriptional or translational control signals may be needed. However, in cases where only coding sequence, or a fragment thereof, is inserted; exogenous translational control signals (including the ATG initiation codon) should be provided. The initiation codon should be in the correct reading frame to ensure translation of the entire insert. Exogenous translational. elements and initiation codons can be of various origins, both natural and synthetic. The efficiency of expression can be enhanced by the inclusion of enhancers which are appropriate for the particular cell system which is used [Scharf et al., 1994, (31)].

### Host Cells

A host cell strain can be chosen for its ability to modulate the expression of the inserted sequences or to process the expressed “BREAST CANCER GENE” polypeptide in the desired fashion. Such modifications of the polypeptide include, but are not limited to, acetylation, carboxylation, glycosylation, phosphorylation, lipidation, and acylation. Posttranslational processing which cleaves a “prepro” form of the polypeptide also can be used to facilitate correct insertion, folding and/or function. Different host cells which have specific cellular machinery and characteristic mechanisms for Post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express “BREAST CANCER GENE” polypeptides can be transformed using expression vectors which can contain viral origins of replication and/or endogenous expression elements and a selectable marker gene on the same or on a separate vector. Following the introduction of the vector, cells can be allowed to grow for 12 days in an enriched medium before they are switched to a selective medium. The purpose of the selectable marker is to confer resistance to selection, and its presence allows growth and recovery of cells which successfully express the introduced “BREAST CANCER GENE” sequences. Resistant clones of stably transformed cells can be proliferated using tissue culture techniques appropriate to the cell type [Freshney et al., 1986, (32).

Any number of selection systems can be used to recover transformed cell lines. These include, but are not limited to, the herpes simplex virus thymidine kinase (Wigler et al., 1977, (33)] and adenine phosphoribosyltransferase [Lowy et al., 1980, (34)] genes which can be employed in tk⁻ or aprt⁻ cells, respectively. Also, antimetabolite, antibiotic, or herbicide resistance can be used as the basis for selection. For example, dhfr confers resistance to methotrexate [Wigler et al., 1980, (35)], npt confers resistance to the aminoglycosides, neomycin and G418 [Colbere-Garapin et al., 1981, (36)], and als and pat confer resistance to chlorsulfuron and phosphinotricin acetyltransferase, respectively. Additional selectable genes have been described. For example, trpB allows cells to utilize indole in place of tryptophan, or hisD, which allows cells to utilize histinol in place of histidine [Hartman & Mulligan, 1988,(37)]. Visible markers such as anthocyanins, β-glucuronidase and its substrate GUS, and luciferase and its substrate luciferin, can be used to identify transformants and to quantify the amount of transient or stable protein expression attributable to a specific vector system [Rhodes et al., 1995, (38)].

### Detecting Expression and Gene Product

Although the presence of marker gene expression suggests that the “BREAST CANCER GENE” polynucleotide is also present, its presence and expression may need to be confirmed. For example, if a sequence encoding a “BREAST CANCER GENE” polypeptide is inserted within a marker gene sequence, transformed cells containing sequences which encode a “BREAST CANCER GENE” polypeptide can be identified by the absence of marker gene function. Alternatively, a marker gene can be placed in tandem with a sequence encoding a “BREAST CANCER GENE” polypeptide under the control of a single promoter. Expression of the marker gene in response to induction or selection usually indicates expression of the “BREAST CANCER GENE” polynucleotide.

Alternatively, host cells which contain a “BREAST CANCER GENE” polynucleotide and which express a “BREAST CANCER GENE” polypeptide can be identified by a variety of procedures known to those of skill in the art. These procedures include, but are not limited to, DNA-DNA or DNA-RNA hybridization and protein bioassay or immunoassay techniques which include membrane, solution, or chip-based technologies for the detection and/or quantification of polynucleotide or protein. For example, the presence of a polynucleotide sequence encoding a “BREAST CANCER GENE” polypeptide can be detected by DNA-DNA or DNA-RNA hybridization or amplification using probes or fragments or fragments of polynucleotides encoding a “BREAST CANCER GENE” polypeptide. Nucleic acid amplification-based assays involve the use of oligonucleotides selected from sequences encoding a “BREAST CANCER GENE” polypeptide to detect transformants which contain a “BREAST CANCER GENE” polynucleotide.

A variety of protocols for detecting and measuring the expression of a “BREAST CANCER GENE” polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a “BREAST CANCER GENE” polypeptide can be used, or a competitive binding assay can be employed. These and other assays are described in Hampton et al., (39) and Maddox et al., 40).

A wide variety of labels and conjugation techniques are known by those skilled in the art and can be used in various nucleic acid and amino acid assays. Means for producing labeled hybridization or PCR probes for detecting sequences related to polynucleotides encoding “BREAST CANCER GENE” polypeptides include oligo labeling, nick translation, end-labeling, or PCR amplification using a labeled nucleotide. Alternatively, sequences encoding a “BREAST CANCER GENE” polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

### Expression and Purification of Polypeptides

Host cells transformed with nucleotide sequences encoding a “BREAST CANCER GENE” polypeptide can be cultured under conditions suitable for the expression and recovery of the protein from cell culture. The polypeptide produced by a transformed cell can be secreted or stored intracellular depending on the sequence and/or the vector used. As will be understood by those of skill in the art, expression vectors containing polynucleotides which encode “BREAST CANCER GENE” polypeptides can be designed to contain signal sequences which direct secretion of soluble “BREAST CANCER GENE” polypeptides through a prokaryotic or eukaryotic cell membrane or which direct the membrane insertion of membrane-bound “BREAST CANCER GENE” polypeptide.

As discussed above, other constructions can be used to join a sequence encoding a “BREAST CANCER GENE” polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enteroidnase (Invitrogen, San Diego, Calif.) between the purification domain and the “BREAST CANCER GENE” polypeptide also can be used to facilitate purification. One such expression vector provides for expression of a fusion protein containing a “BREAST CANCER GENE” polypeptide and 6 histidine residues preceding a thioredoxin or an enterokinase cleavage site. The histidine residues facilitate purification by IMAC (immobilized metal ion affinity chromatography [Porath et al., 1992, (41)], while the enterokinase cleavage site provides a means for purifying the “BREAST CANCER GENE” polypeptide from the fusion protein. Vectors which contain fusion proteins are disclosed in Kroll et al., (42).

### Chemical Synthesis

Sequences encoding a “BREAST CANCER GENE” polypeptide can be synthesized, in whole or in part, using chemical methods well known in the art (see Caruthers et al., (43) and Horn et al., (44). Alternatively, a “BREAST CANCER GENE” polypeptide itself can be produced using chemical methods to synthesize its amino acid sequence, such as by direct peptide synthesis using solid-phase techniques [Merrifield, 1963, (45) and Roberge et al., 1995, (46)]. Protein synthesis can be performed using manual techniques or by automation. Automated synthesis can be achieved, for example, using Applied Biosystems 431A Peptide Synthesizer (Perkin Elmer). Optionally, fragments of “BREAST CANCER GENE” polypeptides can be separately synthesized and combined using chemical methods to produce a full-length molecule.

The newly synthesized peptide can be substantially purified by preparative high performance liquid chromatography [Creighton, 1983, (47)]. The composition of a synthetic “BREAST CANCER GENE” polypeptide can be confirmed by amino acid analysis or sequencing (e.g., the Edman degradation procedure; see Creighton, (47). Additionally, any portion of the amino acid sequence of the “BREAST CANCER GENE” polypeptide can be altered during direct synthesis and/or combined using chemical methods with sequences from other proteins to produce a variant polypeptide or a fusion protein.

### Production of Altered Polypeptides

As will be understood by those of skill in the art, it may be advantageous to produce “BREAST CANCER GENE” polypeptide-encoding nucleotide sequences possessing non-natural occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter “BREAST CANCER GENE” polypeptide-encoding sequences for a variety of reasons, including but not limited to, alterations which modify the cloning, processing, and/or expression of the polypeptide or mRNA product. DNA shuffling by random fragmentation and PCR re-assembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

### Predictive, Diagnostic and Prognostic Assays

The present invention provides compositions, methods, and kits for determining whether a subject is at risk for developing malignant neoplasia and breast cancer in particular by detecting the disclosed biomarkers, i.e., the disclosed polynucleotide markers comprising any of the polynucleotides sequences of the SEQ ID NO 1 to 165 and 472 to 491 and/or the polypeptide markers encoded thereby or polypeptide markers comprising any of the polypeptide sequences of the SEQ ID NO: 166 to 330 and 492 to 511 for malignant neoplasia and breast cancer in particular.

In clinical applications, biological samples can be screened for the presence and/or absence of the biomarkers identified herein. Such samples are for example needle biopsy cores, surgical resection samples, or body fluids like serum, thin needle nipple aspirates and urine. For example, these methods include obtaining a biopsy, which is optionally fractionated by cryostat sectioning to enrich diseases cells to about 80% of the total cell population. In certain embodiments, polynucleotides extracted from these samples may be amplified using techniques well known in the art. The expression levels of selected markers detected would be compared with statistically valid groups of diseased and healthy samples.

In one embodiment the compositions, methods, and kits comprises determining whether a subject has an abnormal mRNA and/or protein level of the disclosed markers, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the levels of the disclosed biomarkers, protein or mRNA level, is determined and compared to the level of these markers in a healthy subject. An abnormal level of the biomarker polypeptide or mRNA levels is likely to be indicative of malignant neoplasia such as breast cancer.

In another embodiment the compositions, methods, and kits comprises determining whether a subject has an abnormal DNA content of said genes or said genomic loci, such as by Southern blot analysis, dot blot analysis, Fluorescence or Colorimetric In Situ Hybridization, Comparative Genomic Hybridization or quantitative PCR. In general these assays comprise the usage of probes from representative genomic regions. The probes contain at least parts of said genomic regions or sequences complementary or analogous to said regions. In particular intra- or intergenic regions of said genes or genomic regions. The probes can consist of nucleotide sequences or sequences of analogous functions (e.g. PNAs, Morpholino oligomers) being able to bind to target regions by hybridization. In general genomic regions being altered in said patient samples are compared with unaffected control samples (normal tissue from the same or different patients, surrounding unaffected tissue, peripheral blood) or with genomic regions of the same sample that don't have said alterations and can therefore serve as internal controls. In a preferred embodiment regions located on the same chromosome are used. Alternatively, gonosomal regions and/or regions with defined varying amount in the sample are used. In one favored embodiment the DNA content, structure, composition or modification is compared that lie within distinct genomic regions. Especially favored are methods that detect the DNA content of said samples, where the amount of target regions are altered by amplification and or deletions. In another embodiment the target regions are analyzed for the presence of polymorphisms (e.g. Single Nucleotide Polymorphisms or mutations) that affect or predispose the cells in said samples with regard to clinical aspects, being of diagnostic, prognostic or therapeutic value. Preferably, the identification of sequence variations is used to define haplotypes that result in characteristic behavior of said samples with said clinical aspects.

In one embodiment, the compositions, methods, and kits for the prediction, diagnosis or prognosis of malignant neoplasia and breast cancer in particular are done by the detection of:
(a) a polynucleotide selected from the polynucleotides of the SEQ ID NO: 1 to 165 and 472 to 491;
(b) a polynucleotide which hybridizes under stringent conditions to a polynucleotide specified in (a) encoding a polypeptide exhibiting the same biological function as specified for the respective sequence in Table 1a and 1b or 4a and 4b;
(c) a polynucleotide the sequence of which deviates from the polynucleotide specified in (a) and (b) due to the generation of the genetic code encoding a polypeptide exhibiting the same biological function as specified for the polypeptides of SEQ ID NO: 166 to 330 and 492 to 511
(d) a polynucleotide which represents a specific fragment, derivative or allelic variation of a polynucleotide sequence specified in (a) to (c) encoding a polypeptide exhibiting the same biological function as specified for the respective sequence in Table 1a and 1b or 4a and 4b;
   in a biological sample comprising the following steps: hybridizing any polynucleotide or analogous oligomer specified in (a) to (d) to a polynucleotide material of a biological sample, thereby forming a hybridization complex; and detecting said hybridization complex.

In another embodiment the method for the prediction, diagnosis or prognosis of malignant neoplasia is done as just described but, wherein before hybridization, the polynucleotide material of the biological sample is amplified.

In another embodiment the method for the diagnosis or prognosis of malignant neoplasia and breast cancer in particular is done by the detection of:
(a) a polynucleotide selected from the polynucleotides of the SEQ ID NO: 166 to 330 and 492 to 511;
(b) a polynucleotide which hybridizes under stringent conditions to a polynucleotide specified in (a) encoding a polypeptide exhibiting the same biological function as specified for the respective sequence in Table 1a and 1b or 4a and 4b;
(c) a polynucleotide the sequence of which deviates from the polynucleotide specified in (a) and (b) due to the generation of the genetic code encoding a polypeptide exhibiting the same biological function as specified for the respective sequence in Table 1a and 1b or 4a and 4b;
(d) a polynucleotide which represents a specific fragment, derivative or allelic variation of a polynucleotide sequence specified in (a) to (c) encoding a polypeptide exhibiting the same biological function as specified for the respective sequence in Table 1a and 1b or 4a and 4b;
(e) a polypeptide encoded by a polynucleotide sequence specified in (a) to (d)
(f) a polypeptide comprising any polypeptide of SEQ 1) NO: 166 to 330 and 492 to 511
(g)
   comprising the steps of contacting a biological sample with a reagent which specifically interacts with the polynucleotide specified in (a) to (d) or the polypeptide specified in (e).

### 1. DNA Array Technology

In one embodiment, the present Invention also provides a method wherein polynucleotide probes are immobilized an a DNA chip in an organized array. Oligonucleotides can be bound to a solid Support by a variety of processes, including lithography. For example a chip can hold up to 410.000 oligonucleotides (GeneChip, Affymetrix). The present invention provides significant advantages over the available tests for malignant neoplasia, such as breast cancer, because it increases the reliability of the test by providing an array of polynucleotide markers an a single chip.

The method includes obtaining a biological sample which can be a biopsy of an affected person, which is optionally fractionated by cryostat sectioning to enrich diseased cells to about 80% of the total cell population and the use of body fluids such as serum or urine, serum or cell containing liquids (e.g. derived from fine needle aspirates). The DNA or RNA is then extracted, amplified, and analyzed with a DNA chip to determine the presence of absence of the marker polynucleotide sequences. In one embodiment, the polynucleotide probes are spotted onto a substrate in a two-dimensional matrix or array. samples of polynucleotides can be labeled and then hybridized to the probes. Double-stranded polynucleotides, comprising the labeled sample polynucleotides bound to probe polynucleotides, can be detected once the unbound portion of the sample is washed away.

The probe polynucleotides can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophores, chromophores, etc. Techniques for constructing arrays and methods of using these arrays are described in EP 0 799 897; WO 97/29212; WO 97/27317; EP 0 785 280; WO 97/02357; U.S. Pat. No. 5,593±839; U.S. Pat. No. 5,578,832; EP 0 728 520; U.S. Pat. No. 5,599,695; EP 0 721 016; U.S. Pat. No. 5,556,752; WO 95/22058; and U.S. Pat. No. 5,631,734. Further, arrays can be used to examine differential expression of genes and can be used to determine gene function. For example, arrays of the instant polynucleotide sequences can be used to determine if any of the polynucleotide sequences are differentially expressed between normal cells and diseased cells, for example. High expression of a particular message in a diseased sample, which is not observed in a corresponding normal sample, can indicate a breast cancer specific protein.

Accordingly, in one aspect, the invention provides probes and primers that are specific to the polynucleotide sequences of SEQ ID NO: 1 to 165 and 472 to 491.

In one embodiment, the composition, method, and kit comprise using a polynucleotide probe to determine the presence of malignant or breast cancer cells in particular in a tissue from a patient. Specifically, the method comprises:
1) providing a polynucleotide probe comprising a nucleotide sequence at least 12 nucleotides in length, preferably at least 15 nucleotides, more preferably, 25 nucleotides, and most preferably at least 40 nucleotides, and up to all or nearly all of the coding sequence which is complementary to a portion of the coding sequence of a polynucleotide selected from the polynucleotides of SEQ ID NO: 1 to 165 and 472 to 491 or a sequence complementary thereto;
2) obtaining a tissue sample from a patient with malignant neoplasia;
3) providing a second tissue sample from a patient with no malignant neoplasia;
4) contacting the polynucleotide probe under stringent conditions with RNA of each of said first and second tissue samples (e.g., in a Northern blot or in situ hybridization assay); and
5) comparing (a) the amount of hybridization of the probe with RNA of the first tissue sample, with (b) the amount of hybridization of the probe with RNA of the second tissue sample;
   wherein a statistically significant difference in the amount of hybridization with the RNA of the first tissue sample as compared to the amount of hybridization with the RNA of the second tissue sample is indicative of malignant neoplasia and breast cancer in particular in the first tissue sample.

### 2. Data Analysis Methods

Comparison of the expression levels of one or more “BREAST CANCER GENES” with reference expression levels, e.g., expression levels in diseased cells of breast cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one “BREAST CANCER GENE” in a diseased cell. The values can be mRNA expression levels obtained from experiments, e.g., microarray analysis. The values can also be mRNA levels normalised relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more “BREAST CANCER GENES” in a first cell, e.g., a cell of a subject, and that in a second cell, comprising obtaining the level of expression of one or more “BREAST CANCER GENES” in a first cell and entering these values into a computer comprising a database including records comprising values corresponding to levels of expression of one or more “BREAST CANCER GENES” in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of “BREAST CANCER GENES” are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises values of expression levels in cells of subjects at different stages of breast cancer, and the computer is capable of comparing expression data entered into the computer with the data stored, and produce results indicating to which of the expression profiles in the computer, the one entered is most similar, such as to determine the stage of breast cancer in the subject.

In yet another embodiment, the reference expression profiles in the computer are expression profiles from cells of breast cancer of one or more subjects, which cells are treated in vivo or in vitro with a drug used for therapy of breast cancer. Upon entering of expression data of a cell of a subject treated in vitro or in vivo with the drug, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of breast cancer in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of breast cancer. The reference cells can also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides a method for selecting a therapy for a patient having breast cancer, the method comprising: (i) providing the level of expression of one or more genes characteristic of breast cancer in a diseased cell of the patient; (ii) providing a plurality of reference profiles, each associated with a therapy, wherein the subject expression profile and each reference profile has a plurality of values, each value representing the level of expression of a gene characteristic of breast cancer; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of an mRNA in two biological samples can be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expression comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of a stage of breast cancer or to a treatment for breast cancer.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputting device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes characteristic of breast cancer. The database may contain one or more expression profiles of genes characteristic of breast cancer in different cells.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

### 3. Detection of Variant Polynucleotide Sequence

In yet another embodiment, the invention provides methods for determining whether a subject is at risk for developing a disease, such as a predisposition to develop malignant neoplasia, for example breast cancer, associated with an aberrant activity of any one of the polypeptides encoded by any of the polynucleotides of the SEQ ID NO: 1 to 165 and 472 to 491, wherein the aberrant activity of the polypeptide is characterized by detecting the presence or absence of a genetic lesion characterized by at least one of these:
(i) an alteration affecting the integrity of a gene encoding a marker polypeptides, or
(ii) the misexpression of the encoding polynucleotide.

To illustrate, such genetic lesions can be detected by ascertaining the existence of at least one of these:
I. a deletion of one or more nucleotides from the polynucleotide sequence
II. an addition of one or more nucleotides to the polynucleotide sequence
III. a substitution of one or more nucleotides of the polynucleotide sequence
IV. a gross chromosomal rearrangement of the polynucleotide sequence
V. a gross alteration in the level of a messenger RNA transcript of the polynucleotide sequence
VI. aberrant modification of the polynucleotide sequence, such as of the methylation pattern of the genomic DNA
VII. the presence of a non-wild type splicing pattern of a messenger RNA transcript of the gene
VIII. a non-wild type level of the marker polypeptide
IX. allelic loss of the gene
X. inappropriate post-translational modification of the marker polypeptide

The present invention provides assay techniques for detecting mutations in the encoding polynucleotide sequence. These methods include, but are not limited to, methods involving sequence analysis, Southern blot hybridization, restriction enzyme site mapping, and methods involving detection of absence of nucleotide pairing between the polynucleotide to be analyzed and a probe.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene in a subject can render the subject susceptible to developing a specific disease or disorder. Polymorphic regions in genes, can be identified, by determining the nucleotide sequence of genes in populations of individuals. If a polymorphic region is identified, then the link with a specific disease can be determined by studying specific populations of individuals, e.g. individuals which developed a specific disease, such as breast cancer. A polymorphic region can be located in any region of a gene, e.g., exons, in coding or non coding regions of exons, introns, and promoter region.

In an exemplary embodiment, there is provided a polynucleotide composition comprising a polynucleotide probe including a region of nucleotide sequence which is capable of hybridising to a sense or antisense sequence of a gene or naturally occurring mutants thereof, or 5′ or 3′ flanking sequences or intronic sequences naturally associated with the subject genes or naturally occurring mutants thereof. The polynucleotide of a cell is rendered accessible for hybridization, the probe is contacted with the polynucleotide of the sample, and the hybridization of the probe to the sample polynucleotide is detected. Such techniques can be used to detect lesions or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridising specifically to allelic variants are attached to a solid phase support, e.g., a “chip”. Mutation detection analysis using these chips comprising oligonucleotides, also termed “DNA probe arrays” is described e.g., in Cronin et al. (48). In one embodiment, a chip comprises all the allelic variants of at least one polymorphic region of a gene. The solid phase support is then contacted with a test polynucleotide and hybridization to the specific probes is detected. Accordingly, the identity of numerous allelic variants of one or more genes can be identified in a simple hybridization experiment.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) [Landegran et al., 1988, (49) and Nakazawa et al., 1994 (50)], the latter of which can be particularly useful for detecting point mutations in the gene; Abravaya et al., 1995, (51)]. In a merely illustrative embodiment, the method includes the steps of (i) collecting a sample of cells from a patient, (ii) isolating polynucleotide (e.g., genomic, mRNA or both) from the cells of the sample, (iii) contacting the polynucleotide sample with one or more primers which specifically hybridize to a polynucleotide sequence under conditions such that hybridization and amplification of the polynucleotide (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication [Guatelli, J. C. et al., 1990, (52)], transcriptional amplification system [Kwoh, D. Y. et al., 1989, (53)], Q-Beta replicase [Lizardi, P. M. et al., 1988, (54)], or any other polynucleotide amplification method, followed by the detection of the amplified molecules using techniques well known to those of skill in the art. These detection schemes are especially useful for the detection of polynucleotide molecules if such molecules are present in very low numbers.

In a preferred embodiment of the subject assay, mutations in, or allelic variants, of a gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover; the use of sequence specific ribozymes (see, for example, U.S. Pat. No. 5,498,531) can be used to score for the presence of specific mutations by development or loss of a ribozyme cleavage site.

### 4. In Situ Hybridization

In one aspect, the method comprises in situ hybridization with a probe derived from a given marker polynucleotide, which sequence is selected from any of the polynucleotide sequences of the SEQ ID NO: 1 to 165 and 472 to 491 or a sequence complementary thereto. The method comprises contacting the labeled hybridization probe with a sample of a given type of tissue from a patient potentially having malignant neoplasia and breast cancer in particular as well as normal tissue from a person with no malignant neoplasia, and determining whether the probe labels tissue of the patient to a degree significantly different (e.g., by at least a factor of two, or at least a factor of five, or at least a factor of twenty, or at least a factor of fifty) than the degree to which normal tissue is labelled.

### Polypeptide Detection

The subject invention further provides a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

### Antibodies

Any type of antibody known in the art can be generated to bind specifically to an epitope of a “BREAST CANCER GENE” polypeptide. An antibody as used herein includes intact immuno-globulin molecules, as well as fragments thereof, such as Fab, F(ab)₂, and Fv, which are capable of binding an epitope of a “BREAST CANCER GENE” polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a “BREAST CANCER GENE” polypeptide can be used therapeutically, as well as in immunochemical assays, such as Western blots, ELISAs, radioimmunoassays, immunohistochemical assays, immunoprecipitations, or other immuno-chemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a “BREAST CANCER GENE” polypeptide provides a detection signal at least 5-, 10-, or 20-fold higher than a detection signal provided with other proteins when used in an immunochemical assay. Preferably, antibodies which specifically bind to “BREAST CANCER GENE” polypeptides do not detect other proteins in immunochemical assays and can immunoprecipitate a “BREAST CANCER GENE” polypeptide from solution.

“BREAST CANCER GENE” polypeptides can be used to immunize a mammal, such as a mouse, rat, rabbit, guinea pig, monkey, or human, to produce polyclonal antibodies. If desired, a “BREAST CANCER GENE” polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a “BREAST CANCER GENE” polypeptide can be prepared using any technique which provides for the production of antibody molecules by continuous cell lines in culture. These techniques include, but are not limited to, the hybridoma technique, the human B cell hybridoma technique, and the EBV hybridoma technique [Kohler et al., 1985, (65); Kozbor et al., 1985, (66); Cote et al., 1983, (67) and Cole et al., 1984, (68)].

In addition, techniques developed for the production of chimeric antibodies, the splicing of mouse antibody genes to human antibody genes to obtain a molecule with appropriate antigen specificity and biological activity, can be used [Morrison et al., 1984, (69); Neuberger et al., 1984, (70); Takeda et al., 1985, (71)]. Monoclonal and other antibodies also can be humanized to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a “BREAST CANCER GENE” polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to “BREAST CANCER GENE” polypeptides. Antibodies with related specificity, but of distinct idiotypic composition, can be generated by chain shuffling from random combinatorial immunoglobulin libraries [Burton, 1991, (72)].

Single-chain antibodies also can be constructed using a DNA amplification method, such as PCR, using hybridoma cDNA as a template [Thirion et al., 1996, (73)]. Single-chain antibodies can be mono- or bispecific, and can be bivalent or tetravalent. Construction of tetravalent, bispecific single-chain antibodies is taught, for example, in Coloma & Morrison, (74). Construction of bivalent, bispecific single-chain antibodies is taught in Mallender & Voss, (75).

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology [Verhaar et al., 1995, (76); Nicholls et al., 1993, (77)].

Antibodies which specifically bind to “BREAST CANCER GENE” polypeptides also can be produced by inducing in vivo production in the lymphocyte population or by screening immunoglobulin libraries or panels of highly specific binding reagents as disclosed in the literature [Orlandi et al., 1989, (789) and Winter et al., 1991, (79)].

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the antibodies described in WO 94/13804, also can be prepared.

Antibodies according to the invention can be purified by methods well known in the art. For example, antibodies can be affinity purified by passage over a column to which a “BREAST CANCER GENE” polypeptide is bound. The bound antibodies can then be eluted from the column using a buffer with a high salt concentration.

Immunoassays are commonly used to quantify the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarisation immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Other methods to quantify the level of a particular protein, or a protein fragment, or modified protein in a particular sample are based on flow-cytometric methods. Flow cytometry allows the identification of proteins on the cell surface as well as of intracellular proteins using fluorochrome labeled, protein specific antibodies or non-labeled antibodies in combination with fluorochrome labeled secondary antibodies. General techniques to be used in performing flow cytometric assays noted above are known to those of ordinary skill in the art. A special method based on the same principles is the microsphere-based flow cytometric. Microsphere beads are labeled with precise quantities of fluorescent dye and particular antibodies. Such techniques are provided by Luminex Inc. WO 97/14028. In another embodiment the level of a particular protein or a protein fragment, or modified protein in a particular sample may be determined by 2D gel-electrophoresis and/or mass spectrometry. Determination of protein nature, sequence, molecular mass as well charge can be achieved in one detection step. Mass spectrometry can be performed with methods known to those with skills in the art as MALDI, TOF, or combinations of these.

In another embodiment, the level of the encoded product, i.e., the product encoded by any of the polynucleotide sequences of the SEQ ID NO: 1 to 165 and 472 to 491 or a sequence complementary thereto, in a biological fluid (e.g., blood or urine) of a patient may be determined as a way of monitoring the level of expression of the marker polynucleotide sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of the disorder, e.g., plaque formation. The level of the marker polypeptide can be used predictively to evaluate whether a sample of cells contains cells which are, or are predisposed towards becoming, plaque associated cells. The observation of marker polypeptide level can be utilized in decisions regarding, e.g., the use of more stringent therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantify the level of marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

### Immunohistochemistry

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissues samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a Label for subsequent detection of binding. samples are incubated for a time Sufficient for formation of the immunocomplexes. Binding of the antibody is then detected by virtue of a Label conjugated to this antibody. Where the antibody is unlabelled, a second labeled antibody may be employed, e.g., which is specific for the isotype of the anti-marker polypeptide antibody. Examples of labels which may be employed include radionuclides, fluorescence, chemoluminescence, and enzymes.

Where enzymes are employed, the Substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination of the average amount of the marker polypeptide associated with a Single cell by correlating the amount of marker polypeptide in a cell-free extract produced from a predetermined number of cells.

In yet another embodiment, the invention contemplates using a panel of antibodies which are generated against the marker polypeptides of this invention, which polypeptides are encoded by any of the polynucleotide sequences of the SEQ ID NO: 1 to 165 and 472 to 491. Such a panel of antibodies may be used as a reliable diagnostic probe for breast cancer. The assay of the present invention comprises contacting a biopsy sample containing cells, e.g., macrophages, with a panel of antibodies to one or more of the encoded products to determine the presence or absence of the marker polypeptides.

The diagnostic methods of the subject invention may also be employed as follow-up to treatment, e.g., quantification of the level of marker polypeptides may be indicative of the effectiveness of current or previously employed therapies for malignant neoplasia and breast cancer in particular as well as the effect of these therapies upon patient prognosis.

The diagnostic assays described above can be adapted to be used as prognostic assays, as well. Such an application takes advantage of the sensitivity of the assays of the Invention to events which take place at characteristic stages in the progression of plaque generation in case of malignant neoplasia. For example, a given marker gene may be up- or down-regulated at a very early stage, perhaps before the cell is developing into a foam cell, while another marker gene may be characteristically up or down regulated only at a much later stage. Such a method could involve the steps of contacting the mRNA of a test cell with a polynucleotide probe derived from a given marker polynucleotide which is expressed at different characteristic levels in breast cancer tissue cells at different stages of malignant neoplasia progression, and determining the approximate amount of hybridization of the probe to the mRNA of the cell, such amount being an indication of the level of expression of the gene in the cell, and thus an indication of the stage of disease progression of the cell; alternatively, the assay can be carried out with an antibody specific for the gene product of the given marker polynucleotide, contacted with the proteins of the test cell. A battery of such tests will disclose not only the existence of a certain neoplastic lesion, but also will allow the clinician to select the mode of treatment most appropriate for the disease, and to predict the likelihood of success of that treatment.

The methods of the invention can also be used to follow the clinical course of a given breast cancer predisposition. For example, the assay of the Invention can be applied to a blood sample from a patient; following treatment of the patient for BREAST CANCER, another blood sample is taken and the test repeated. Successful treatment will result in removal of demonstrate differential expression, characteristic of the breast cancer tissue cells, perhaps approaching or even surpassing normal levels.

### Polypeptide Activity

In one embodiment the present invention provides a method for screening potentially therapeutic agents which modulate the activity of one or more “BREAST CANCER GENE” polypeptides, such that if the activity of the polypeptide is increased as a result of the upregulation of the “BREAST CANCER GENE” in a subject having or at risk for malignant neoplasia and breast cancer in particular, the therapeutic substance will decrease the activity of the polypeptide relative to the activity of the some polypeptide in a subject not having or not at risk for malignant neoplasia or breast cancer in particular but not treated with the therapeutic agent. Likewise, if the activity of the polypeptide as a result of the downregulation of the “BREAST CANCER GENE” is decreased in a subject having or at risk for malignant neoplasia or breast cancer in particular, the therapeutic agent will increase the activity of the polypeptide relative to the activity of the same polypeptide in a subject not having or not at risk for malignant neoplasia or breast cancer in particular, but not treated with the therapeutic agent.

The activity of the “BREAST CANCER GENE” polypeptides indicated in Table 2 or 3 may be measured by any means known to those of skill in the art, and which are particular for the type of activity performed by the particular polypeptide. Examples of specific assays which may be used to measure the activity of particular polynucleotides are shown below.

### a) G Protein Coupled Receptors

In one embodiment, the “BREAST CANCER GENE” polynucleotide may encode a G protein coupled receptor. In one embodiment, the present invention provides a method of screening potential modulators (inhibitors or activators) of the G protein coupled receptor by measuring changes in the activity of the receptor in the presence of a candidate modulator.

### 1) Gᵢ-Coupled Receptors

Cells (such as CHO cells or primary cells) are stably transfected with the relevant receptor and with an inducible CRE-luciferase construct. Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 2000 cells/well in 35 μl cell culture medium) in DMEM/F12 with FBS, and are grown for 48 hours (range: ˜24-60 hours, depending on cell line). Growth medium is then exchanged against serum free medium (SFM; e.g. Ultra-CHO), containing 0.1% BSA. Test compounds dissolved in DMSO are diluted in SFM and transferred to the test cultures (maximal final concentration 10 μmolar), followed by addition of forskolin (˜1 μmolar, final conc.) in SFM+0.1% BSA 10 minutes later. In case of antagonist screening both, an appropriate concentration of agonist, and forskolin are added. The plates are incubated at 37° C. in 10% CO₂ for 3 hours. Then the supernatant is removed, cells are lysed with lysis reagent (25 mmolar phosphate-buffer, pH 7.8, containing 2 mmolar DDT, 10% glycerol and 3% Triton X100). The luciferase reaction is started by addition of substrate-buffer (e.g. luciferase assay reagent, Promega) and luminescence is immediately determined (e.g. Berthold luminometer or Hamamatzu camera system).

### 2) Gᵢ-Coupled Receptors

Cells (such as CHO cells or primary cells) are stably transfected with the relevant receptor and with an inducible CRE-luciferase construct. Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 1000 or 2000 cells/well in 35 μl cell culture medium) in DMEM/F12 with FBS, and are grown for 48 hours (range: ˜24-60 hours, depending on cell line). The assay is started by addition of test-compounds in serum free medium (SFM; e.g. Ultra-CHO) containing 0.1% BSA: Test compounds are dissolved in DMSO, diluted in SFM and transferred to the test cultures (maximal final concentration 10 μmolar, DMSO conc.<0.6%). In case of antagonist screening an appropriate concentration of agonist is added 5-10 minutes later. The plates are incubated at 37° C. in 10% CO₂ for 3 hours. Then the cells are lysed with 10 μl lysis reagent per well (25 mmolar phosphate-buffer, pH 7.8, containing 2 mmolar DDT, 10% glycerol and 3% Triton X100) and the luciferase reaction is started by addition of 20 μl substrate-buffer per well (e.g. luciferase assay reagent, Promega). Measurement of luminescence is started immediately (e.g. Berthold luminometer or Hamamatzu camera system).

### 3) G_{q}-Coupled Receptors

Cells (such as CHO cells or primary cells) are stably transfected with the relevant receptor. Cells expressing functional receptor protein are grown in 50% Dulbecco's modified Eagle medium/50% F12 (DMEM/F12) supplemented with 10% FBS, at 37° C. in a humidified atmosphere with 5% CO₂ and are routinely split at a cell line dependent ratio every 3 or 4 days. Test cultures are seeded into 384-well plates at an appropriate density (e.g. 2000 cells I well in 35 μl cell culture medium) in DMEM/F12 with FBS, and are grown for 48 hours (range: ˜24-60 hours, depending on cell line). Growth medium is then exchanged against physiological salt solution (e.g. Tyrode solution). Test compounds dissolved in DMSO are diluted in Tyrode solution containing 0.1% BSA and transferred to the test cultures (maximal final concentration 10 μmolar). After addition of the receptor specific agonist the resulting Gq-mediated intracellular calcium increase is measured using appropriate read-out systems (e.g. calcium-sensitive dyes).

### b) Ion Channels

Ion channels are integral membrane proteins involved in electrical signaling, transmembrane signal transduction, and electrolyte and solute transport. By forming macromolecular pores through the membrane lipid bilayer, ion channels account for the flow of specific ion species driven by the electrochemical potential gradient for the permeating ion. At the single molecule level, individual channels undergo conformational transitions (“gating”) between the ‘open’ (ion conducting) and ‘closed’ (non conducting) state. Typical single channel openings last for a few milliseconds and result in elementary transmembrane currents in the range of 10⁻⁹-10⁻¹² Ampere. Channel gating is controlled by various chemical and/or biophysical parameters, such as neurotransmitters and intracellular second messengers (‘ligand-gated’ channels) or membrane potential (‘voltage-gated’ channels). Ion channels are functionally characterized by their ion selectivity, gating properties, and regulation by hormones and pharmacological agents. Because of their central role in signaling and transport processes, ion channels present ideal targets for pharmacological therapeutics in various pathophysiological settings.

In one embodiment, the “BREAST CANCER GENE” may encode an ion channel. In one embodiment, the present invention provides a method of screening potential activators or inhibitors of channels activity of the “BREAST CANCER GENE” polypeptide. Screening for compounds interaction with ion channels to either inhibit or promote their activity can be based on (1.) binding and (2.) functional assays in living cells [Hille (112)].
1. For ligand-gated channels, e.g. ionotropic neurotransmitter/hormone receptors, assays can be designed detecting binding to the target by competition between the compound and a labeled ligand.
2. Ion channel function can be tested functionally in living cells. Target proteins are either expressed endogenously in appropriate reporter cells or are introduced recombinantly. Channel activity can be monitored by (2.1) concentration changes of the permeating ion (most prominently Ca²⁺ ions), (2.2) by changes in the transmembrane electrical potential gradient, and (2.3) by measuring a cellular response (e.g. expression of a reporter gene, secretion of a neurotransmitter) triggered or modulated by the target activity.
   2.1 Channel activity results in transmembrane ion fluxes. Thus activation of ionic channels can be monitored by the resulting changes in intracellular ion concentrations using luminescent or fluorescent indicators. Because of its wide dynamic range and availability of suitable indicators this applies particularly to changes in intracellular Ca²⁺ ion concentration ([Ca²⁺]ᵢ). [Ca²⁺]; can be measured, for example, by aequorin luminescence or fluorescence dye technology (e.g. using Fluo-3, Indo-1, Fura-2). Cellular assays can be designed where either the Ca²⁺ flux through the target channel itself is measured directly or where modulation of the target channel affects membrane potential and thereby the activity of co-expressed voltage-gated Ca²⁺ channels.
   2.2 Ion channel currents result in changes of electrical membrane potential (Vₘ) which can be monitored directly using potentiometric fluorescent probes. These electrically charged indicators (e.g. the anionic oxonol dye DiBAC₄(3)) redistribute between extra- and intracellular compartment in response to voltage changes. The equilibrium distribution is governed by the Nernst-equation. Thus changes in membrane potential results in concomitant changes in cellular fluorescence. Again, changes in Vₘ might be caused directly by the activity of the target ion channel or through amplification and/or prolongation of the signal by channels co-expressed in the same cell.
   2.3 Target channel activity can cause cellular Ca²⁺ entry either directly or through activation of additional Ca²⁺ channel (see 2.1). The resulting intracellular Ca²⁺ signals regulate a variety of cellular responses, e.g. secretion or gene transcription. Therefore modulation of the target channel can be detected by monitoring secretion of a known hormone/transmitter from the target-expressing cell or through expression of a reporter gene (e.g. luciferase) controlled by an Ca²⁺-responsive promoter element (e.g. cyclic AMP/Ca²⁺-responsive elements; CRE).

### c) DNA-Binding Proteins and Transcription Factors

In one embodiment, the “BREAST CANCER GENE” may encode a DNA-binding protein or a transcription factor. The activity of such a DNA-binding protein or a transcription factor may be measured, for example, by a promoter assay which measures the ability of the DNA-binding protein or the transcription factor to initiate transcription of a test sequence linked to a particular promoter. In one embodiment, the present invention provides a method of screening test compounds for its ability to modulate the activity of such a DNA-binding protein or a transcription factor by measuring the changes in the expression of a test gene which is regulated by a promoter which is responsive to the transcription factor.

### Promotor Assays

A promoter assay was set up with a human hepatocellular carcinoma cell HepG2 that was stably transfected with a luciferase gene under the control of a gene of interest (e.g. thyroid hormone) regulated promoter. The vector 2×IROluc, which was used for transfection, carries a thyroid hormone responsive element (TRE) of two 12 bp inverted palindromes separated by an 8 bp spacer in front of a tk minimal promoter and the luciferase gene. Test cultures were seeded in 96 well plates in serum-free Eagle's Minimal Essential Medium supplemented with glutamine, tricine, sodium pyruvate, non-essential amino acids, insulin, selen, transferrin, and were cultivated in a humidified atmosphere at 10% CO₂ at 37° C. After 48 hours of incubation serial dilutions of test compounds or reference compounds (L-T3, L-T4 e.g.) and co-stimulator if appropriate (final concentration 1 nM) were added to the cell cultures and incubation was continued for the optimal time (e.g. another 4-72 hours). The cells were then lysed by addition of buffer containing Triton X100 and luciferin and the luminescence of luciferase induced by T3 or other compounds was measured in a luminometer. For each concentration of a test compound replicates of 4 were tested. EC₅₀-values for each test compound were calculated by use of the Graph Pad Prism Scientific software.

### Screening Methods

The invention provides assays for screening test compounds which bind to or modulate the activity of a “BREAST CANCER GENE” polypeptide or a “BREAST CANCER GENE” polynucleotide. A test compound preferably binds to a “BREAST CANCER GENE” polypeptide or polynucleotide. More preferably, a test compound decreases or increases “BREAST CANCER GENE” activity by at least about 10, preferably about 50, more preferably about 75, 90, or 100% relative to the absence of the test compound.

### Test Compounds

Test compounds can be pharmacological agents already known in the art or can be compounds previously unknown to have any pharmacological activity. The compounds can be naturally occurring or designed in the laboratory. They can be isolated from microorganisms, animals, or plants, and can be produced recombinant, or synthesised by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the one-bead one-compound library method, and synthetic library methods using affinity chromatography selection. The biological library approach is limited to polypeptide libraries, while the other four approaches are applicable to polypeptide, non-peptide oligomer, or small molecule libraries of compounds. [For review see Lam, 1997, (80)].

Methods for the synthesis of molecular libraries are well known in the art [see, for example, DeWitt et al., 1993, (81); Erb et al., 1994, (82); Zuckermann et al., 1994, (83); Cho et al., 1993, (84); Carell et al., 1994, (85) and Gallop et al., 1994, (86). Libraries of compounds can be presented in solution [see, e.g., Houghten, 1992, (87)], or on beads [Lam, 1991, (88)], DNA-chips [Fodor, 1993, (89)], bacteria or spores (Ladner, U.S. Pat. No. 5,223,409), plasmids [Cull et al., 1992, (901)], or phage [Scott & Smith, 1990, (91); Devlin, 1990, (92); Cwirla et al., 1990, (93); Felici, 1991, (94)].

High Throughput Screening

Test compounds can be screened for the ability to bind to “BREAST CANCER GENE” polypeptides or polynucleotides or to affect “BREAST CANCER GENE” activity or “BREAST CANCER GENE” expression using high throughput screening. Using high throughput screening, many discrete compounds can be tested in parallel so that large numbers of test compounds can be quickly screened. The most widely established techniques utilize 96-well, 384-well or 1536-well microtiter plates. The wells of the microtiter plates typically require assay volumes that range from 5 to 500 μl. In addition to the plates, many instruments, materials, pipettors, robotics, plate washers, and plate readers are commercially available to fit the microwell formats.

Alternatively, free format assays, or assays that have no physical barrier between samples, can be used. For example, an assay using pigment cells (melanocytes) in a simple homogeneous assay for combinatorial peptide libraries is described by Jayawickreme et al., (95). The cells are placed under agarose in culture dishes, then beads that carry combinatorial compounds are placed on the surface of the agarose. The combinatorial compounds are partially released the compounds from the beads. Active compounds can be visualised as dark pigment areas because, as the compounds diffuse locally into the gel matrix, the active compounds cause the cells to change colors.

Another example of a free format assay is described by Chelsky, (96). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV light. Compounds that inhibited the enzyme were observed as local zones of inhibition having less color change.

In another example, combinatorial libraries were screened for compounds that had cytotoxic effects on cancer cells growing in agar [Salmon et al., 1996, (97)].

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

### Binding Assays

For binding assays, the test compound is preferably a small molecule which binds to and occupies, for example, the ATP/GTP binding site of the enzyme or the active site of a “BREAST CANCER GENE” polypeptide, such that normal biological activity is prevented. Examples of such small molecules include, but are not limited to, small peptides or peptide-like molecules.

In binding assays, either the test compound or a “BREAST CANCER GENE” polypeptide can comprise a detectable label, such as a fluorescent, radioisotopic, chemiluminescent, or enzymatic label, such as horseradish peroxidase, alkaline phosphatase, or luciferase. Detection of a test compound which is bound to a “BREAST CANCER GENE” polypeptide can then be accomplished, for example, by direct counting of radioemmission, by scintillation counting, or by determining conversion of an appropriate substrate to a detectable product.

Alternatively, binding of a test compound to a “BREAST CANCER GENE” polypeptide can be determined without labeling either of the interactants. For example, a microphysiometer can be used to detect binding of a test compound with a “BREAST CANCER GENE” polypeptide. A microphysiometer (e.g., CytosensorJ) is an analytical instrument that measures the rate at which a cell acidifies its environment using a light-addressable potentiometric sensor (LAPS). Changes in this acidification rate can be used as an indicator of the interaction between a test compound and a “BREAST CANCER GENE” polypeptide [McConnell et al., 1992, (98)].

Determining the ability of a test compound to bind to a “BREAST CANCER GENE” polypeptide also can be accomplished using a technology such as real-time Bimolecular Interaction Analysis (BIA) [Sjolander & Urbaniczky, 1991, (99), and Szabo et al., 1995, (100)]. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a “BREAST CANCER GENE” polypeptide can be used as a “bait protein” in a two-hybrid assay or three-hybrid assay [see, e.g., U.S. Pat. No. 5,283,317; Zervos et al., 1993, (101); Madura et al., 1993, (102); Bartel et al., 1993, (1034); Iwabuchi et al., 1993, (104) and Brent WO 94/10300], to identify other proteins which bind to or interact with the “BREAST CANCER GENE” polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a “BREAST CANCER GENE” polypeptide can be fused to a polynucleotide encoding the DNA binding domain of a known transcription factor (e.g., GAL4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the “BREAST CANCER GENE” polypeptide.

It may be desirable to immobilize either a “BREAST CANCER GENE” polypeptide (or polynucleotide) or the test compound to facilitate separation of bound from unbound forms of one or both of the interactants, as well as to accommodate automation of the assay. Thus, either a “BREAST CANCER GENE” polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach a “BREAST CANCER GENE” polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a “BREAST CANCER GENE” polypeptide (or polynucleotide) can be accomplished in any vessel suitable for containing the reactants. Examples of such vessels include microtiter plates, test tubes, and microcentrifuge tubes.

In one embodiment, a “BREAST CANCER GENE” polypeptide is a fusion protein comprising a domain that allows the “BREAST CANCER GENE” polypeptide to be bound to a solid support. For example, glutathione S-transferase fusion proteins can be adsorbed onto glutathione sepharose beads (Sigma Chemical, St. Louis, Mo.) or glutathione derivatized microtiter plates, which are then combined with the test compound or the test compound and the nonadsorbed “BREAST CANCER GENE” polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilising proteins or polynucleotides on a solid support also can be used in the screening assays of the invention. For example, either a “BREAST CANCER GENE” polypeptide (or polynucleotide) or a test compound can be immobilized utilizing conjugation of biotin and streptavidin. Biotinylated “BREAST CANCER GENE” polypeptides (or polynucleotides) or test compounds can be prepared from biotin NHS (N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a “BREAST CANCER GENE” polypeptide, polynucleotide, or a test compound, but which do not interfere with a desired binding site, such as the ATP/GTP binding site or the active site of the “BREAST CANCER GENE” polypeptide, can be derivatised to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to a “BREAST CANCER GENE” polypeptide or test compound, enzyme-linked assays which rely on detecting an activity of a “BREAST CANCER GENE” polypeptide, and SDS gel electrophoresis under non-reducing conditions.

Screening for test compounds which bind to a “BREAST CANCER GENE” polypeptide or polynucleotide also can be carried out in an intact cell. Any cell which comprises a “BREAST CANCER GENE” polypeptide or polynucleotide can be used in a cell-based assay system. A “BREAST CANCER GENE” polynucleotide can be naturally occurring in the cell or can be introduced using techniques such as those described above. Binding of the test compound to a “BREAST CANCER GENE” polypeptide or polynucleotide is determined as described above.

### Modulation of Gene Expression

In another embodiment, test compounds which increase or decrease “BREAST CANCER GENE” expression are identified. A “BREAST CANCER GENE” polynucleotide is contacted with a test compound in an appropriate expression test system as described below or in a cell system, and the expression of an RNA or polypeptide product of the “BREAST CANCER GENE” polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of “BREAST CANCER GENE” mRNA or polypeptide expression in the cells can be determined by methods well known in the art for detecting mRNA or polypeptide. Either qualitative or quantitative methods can be used. The presence of polypeptide products of a “BREAST CANCER GENE” polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a “BREAST CANCER GENE” polypeptide.

Such screening can be carried out either in a cell-free assay system or in an intact cell. Any cell which expresses a “BREAST CANCER GENE” polynucleotide can be used in a cell-based assay system. A “BREAST CANCER GENE” polynucleotide can be naturally occurring in the cell or can be introduced using techniques such as those described above. Either a primary culture or an established cell line, such as CHO or human embryonic kidney 293 cells, can be used.

### Therapeutic Indications and Methods

Therapies for treatment of breast cancer primarily relied upon effective chemotherapeutic drugs for intervention on the cell proliferation, cell growth or angiogenesis. The advent of genomics-driven molecular target identification has opened up the possibility of identifying new breast cancer-specific targets for therapeutic intervention that will provide safer, more effective treatments for malignant neoplasia patients and breast cancer patients in particular. Thus, newly discovered breast cancer-associated genes and their products can be used as tools to develop innovative therapies. The identification of the Her2/neu receptor kinase presents exciting new opportunities for treatment of a certain subset of tumor patients as described before. Genes playing important roles in any of the physiological processes outlined above can be characterized as breast cancer targets. Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Modulators of target gene expression or protein activity can be identified in this manner and subsequently tested in cellular and in vivo disease models for therapeutic activity. Optimization of lead compounds with iterative testing in biological models and detailed pharmacokinetic and toxicological analyses form the basis for drug development and subsequent testing in humans.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense polynucleotide molecule, a specific antibody, ribozyme, or a human “BREAST CANCER GENE” polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above described screening assays for treatments as described herein.

A reagent which affects human “BREAST CANCER GENE” activity can be administered to a human cell, either in vitro or in vivo, to reduce or increase human “BREAST CANCER GENE” activity. The reagent preferably binds to an expression product of a human “BREAST CANCER GENE”. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmol of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes usually used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al., 1993, (105); Chiou et al., 1994, (106); Wu & Wu, 1988, (107); Wu et al., 1994, (108); Zenke et al., 1990, (109); Wu et al., 1991, (110).

### Determination of a Therapeutically Effective Dose

The determination of a therapeutically effective dose is well within the capability of those skilled in the art. A therapeutically effective dose refers to that amount of active ingredient which increases or decreases human “BREAST CANCER GENE” activity relative to the human “BREAST CANCER GENE” activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, a gene gun, and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a “BREAST CANCER GENE” gene or the activity of a “BREAST CANCER GENE” polypeptide by at least about 10, preferably about 50, more preferably about 75, 90, or 100% relative to the absence of the reagent. The effectiveness of the mechanism chosen to decrease the level of expression of a “BREAST CANCER GENE” gene or the activity of a “BREAST CANCER GENE” polypeptide can be assessed using methods well known in the art, such as hybridization of nucleotide probes to “BREAST CANCER GENE”-specific mRNA, quantitative RT-PCR, immunologic detection of a “BREAST CANCER GENE” polypeptide, or measurement of “BREAST CANCER GENE” activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, birds and mammals such as dogs, cats, cows, pigs, sheep, goats, horses, rabbits, monkeys, and most preferably, humans.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### Pharmaceutical Compositions

The invention also provides pharmaceutical compositions which can be administered to a patient to achieve a therapeutic effect. Pharmaceutical compositions of the invention can comprise, for example, a “BREAST CANCER GENE” polypeptide, “BREAST CANCER GENE” polynucleotide, ribozymes or antisense oligonucleotides, antibodies which specifically bind to a “BREAST CANCER GENE” polypeptide, or mimetics, agonists, antagonists, or inhibitors of a “BREAST CANCER GENE” polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intraarterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth; and proteins such as gelatin and collagen. If desired, disintergrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 150 mM histidine, 0.1%2% sucrose, and 27% mannitol, at a pH range of 4.5 to 5.5, that is combined with buffer prior to use.

Further details on techniques for formulation and administration can be found in the latest edition of REMINGTON'S PHARMACEUTICAL SCIENCES (111). After pharmaceutical compositions have been prepared, they can be placed in an appropriate container and labeled for treatment of an indicated condition. Such labeling would include amount, frequency, and method of administration.

One strategy for identifying genes that are involved in breast cancer is to detect genes that are expressed differentially under conditions associated with the disease versus non-disease or in the context of therapy response conditions. The sub-sections below describe a number of experimental systems which can be used to detect such differentially expressed genes. In general, these experimental systems include at least one experimental condition in which subjects or samples are treated in a manner associated with breast cancer, in addition to at least one experimental control condition lacking such disease associated treatment or does not respond to such treatment. Differentially expressed genes are detected, as described below, by comparing the pattern of gene expression between the experimental and control conditions.

Once a particular gene has been identified through the use of one such experiment, its expression pattern may be further characterized by studying its expression in a different experiment and the findings may be validated by an independent technique. Such use of multiple experiments may be useful in distinguishing the roles and relative importance of particular genes in breast cancer and the treatment thereof. A combined approach, comparing gene expression pattern in cells derived from breast cancer patients to those of in vitro cell culture models can give substantial hints on the pathways involved in development and/or progression of breast cancer. It can also elucidate the role of such genes in the development of resistance or insensitivity to certain therapeutic agents (e.g. chemotherapeutic drugs).

Among the experiments which may be utilized for the identification of differentially expressed genes involved in malignant neoplasia and breast cancer in particular, are experiments designed to analyze those genes which are involved in signal transduction. Such experiments may serve to identify genes involved in the proliferation of cells.

Below are methods described for the identification of genes which are involved in breast cancer. Such represent genes which are differentially expressed in breast cancer conditions relative to their expression in normal, or non-breast cancer conditions or upon experimental manipulation based on clinical observations. Such differentially expressed genes represent “target” and/or “marker” genes. Methods for the further characterization of such differentially expressed genes, and for their identification as target and/or marker genes, are presented below.

Alternatively, a differentially expressed gene may have its expression modulated, i.e., quantitatively increased or decreased, in normal versus breast cancer states, or under control versus experimental conditions. The degree to which expression differs in normal versus breast cancer or control versus experimental states need only be large enough to be visualized via standard characterization techniques, such as, for example, the differential display technique described below. Other such standard characterization techniques by which expression differences may be visualized include but are not limited to quantitative RT-PCR and Northern analyses, which are well known to those of skill in the art.

In Addition to the experiments described above the following describes algorithms and statistical analyses which can be utilized for data evaluation and for the classification as well as response prediction for a so far not classified biological sample in the context of control samples. Predictive algorithms and equations described below have already shown their power to subdivide individual cancers.

### EXAMPLE 1

### Expression Profiling Utilizing Quantitative Kinetic RT-PCR

For a detailed analysis of gene expression by quantitative PCR methods, one will utilize primers flanking the genomic region of interest and a fluorescent labeled probe hybridizing in-between. Using the PRISM 7700 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly in the 3′ region of the coding sequence or in the 3′ untranslated region (see Table 5 for primer- and probe-sequences). All primer pairs were checked for specificity by conventional PCR reactions and gel electrophoresis. To standardize the amount of sample RNA, GAPDH was selected as a reference, since it was not differentially regulated in the samples analyzed. To perform such an expression analysis of genes within a biological samples the respective primer/probes are prepared by mixing 25 μl of the 100 μM stock solution “Upper Primer”, 25 μl of the 100 μM stock solution “Lower Primer” with 12.5 μl of the 100 μM stock solution TaqMan-probe (FAM/Tamra) and adjusted to 500 μl with aqua dest (Primer/probe-mix). For each reaction 1.25 μl cDNA of the patient samples were mixed with 8.75 μl nuclease-free water and added to one well of a 96 Well-Optical Reaction Plate (Applied Biosystems Part No. 4306737). 1.5 μl of the Primer/Probe-mix described above, 12.5 μl Taq Man Universal-PCR-mix (2×) (Applied Biosystems Part No. 4318157) and 1 μl Water are then added. The 96 well plates are closed with 8 Caps/Strips (Applied Biosystems Part Number 4323032) and centrifuged for 3 minutes. Measurements of the PCR reaction are done according to the instructions of the manufacturer with a TaqMan 7900 HT from Applied Biosystems (No. 20114) under appropriate conditions (2 min. 50° C., 10 min. 95° C., 0.15 min. 95° C., 1 min. 60° C.; 40 cycles). Prior to the measurement of so far unclassified biological samples control experiments will e.g. cell lines, healthy control samples, samples of defined therapy response could be used for standardization of the experimental conditions.

TaqMan validation experiments were performed showing that the efficiencies of the target and the control amplifications are approximately equal which is a prerequisite for the relative quantification of gene expression by the comparative ΔΔC_{T} method, known to those with skills in the art. Herefor the SoftwareSDS 2.0 from Applied Biosystems can be used according to the respective instructions. CT-values are then further analyzed with appropriate software (Microsoft Excel™) of statistical software packages (SAS).

As well as the technology described above, provided by Perkin Elmer, one may use other technique implementations like Lightcycler™ from Roche Inc. or iCycler from Stratagene Inc. capable of real time detection of an RT-PCR reaction.

### EXAMPLE 2

### Expression Profiling Utilizing DNA Microarrays

Expression profiling can bee carried out using the Affymetrix Array Technology. By hybridization of mRNA to such a DNA-array or DNA-Chip, it is possible to identify the expression value of each transcripts due to signal intensity at certain position of the array. Usually these DNA-arrays are produced by spotting of cDNA, oligonucleotides or subcloned DNA fragments. In case of Affymetrix technology app. 400.000 individual oligonucleotide sequences were synthesized on the surface of a silicon wafer at distinct positions. The minimal length of oligomers is 12 nucleotides, preferable 25 nucleotides or full length of the questioned transcript. Expression profiling may also be carried out by hybridization to nylon or nitro-cellulose membrane bound DNA or oligonucleotides. Detection of signals derived from hybridization may be obtained by either colorimetric, fluorescent, electrochemical, electronic, optic or by radioactive readout. Detailed description of array construction have been mentioned above and in other patents cited. To determine the quantitative and qualitative changes in the chromosomal region to analyze, RNA from tumor tissue which is suspected to contain such genomic alterations has to be compared to RNA extracted from benign tissue (e.g. epithelial breast tissue, or micro dissected ductal tissue) on the basis of expression profiles for the whole transcriptome. With minor modifications, the sample preparation protocol followed the Affymetrix GeneChip Expression Analysis Manual (Santa Clara, Calif.). Total RNA extraction and isolation from tumor or benign tissues, biopsies, cell isolates or cell containing body fluids can be performed by using TRIzol (Life Technologies, Rockville, Md.) and Oligotex mRNA Midi kit (Qiagen, Hilden, Germany), and an ethanol precipitation step should be carried out to bring the concentration to 1 mg/ml. Using 5-10 mg of mRNA to create double stranded cDNA by the SuperScript system (Life Technologies). First strand cDNA synthesis was primed with a T7-(dT24) oligonucleotide. The cDNA can be extracted with phenol/chloroform and precipitated with ethanol to a final concentration of 1 mg/ml. From the generated cDNA, cRNA can be synthesized using Enzo's (Enzo Diagnostics Inc., Farmingdale, N.Y.) in vitro Transcription Kit. Within the same step the cRNA can be labeled with biotin nucleotides Bio-11-CTP and Bio-16-UTP (Enzo Diagnostics Inc., Farmingdale, N.Y.). After labeling and cleanup (Qiagen, Hilden (Germany) the cRNA then should be fragmented in an appropriated fragmentation buffer (e.g., 40 mM Tris-Acetate, pH 8.1, 100 mM KOAc, 30 mM MgOAc, for 35 minutes at 94° C.). As per the Affymetrix protocol, fragmented cRNA should be hybridized on the HG_U133 arrays A and B, comprising app. 40.000 probed transcripts each, for 24 hours at 60 rpm in a 45° C. hybridization oven. After Hybridization step the chip surfaces have to be washed and stained with streptavidin phycoerythrin (SAPE; Molecular Probes, Eugene, Oreg.) in Affymetrix fluidics stations. To amplify staining, a second labeling step can be introduced, which is recommended but not compulsive. Here one should add SAPE solution twice with an antistreptavidin biotinylated antibody. Hybridization to the probe arrays may be detected by fluorometric scanning (Hewlett Packard Gene Array Scanner; Hewlett Packard Corporation, Palo Alto, Calif.).

After hybridization and scanning, the microarray images can be analyzed for quality control, looking for major chip defects or abnormalities in hybridization signal. Therefor either Affymetrix GeneChip MAS 5.0 Software or other microarray image analysis software can be utilized. Primary data analysis should be carried out by software provided by the manufacturer.

In case of the genes analyses in one embodiment of this invention the primary data have been analyzed by further bioinformatic tools and additional filter criteria. The bioinformatic analysis is described in detail below.

### EXAMPLE 3

### Data Analysis from Expression Profiling Experiments

According to Affymetrix measurement technique (Affymetrix GeneChip Expression Analysis Manual, Santa Clara, Calif.) a single gene expression measurement on one chip yields the average difference value and the absolute call. Each chip contains 16-20 oligonucleotide probe pairs per gene or cDNA clone. These probe pairs include perfectly matched sets and mismatched sets, both of which are necessary for the calculation of the average difference, or expression value, a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The average difference is a numeric value supposed to represent the expression value of that gene. The absolute call can take the values ‘A’ (absent), ‘M’ (marginal), or ‘P’ (present) and denotes the quality of a single hybridization. We used both the quantitative information given by the average difference and the qualitative information given by the absolute call to identify the genes which are differentially expressed in biological samples from individuals with breast cancer versus biological samples from the normal population. With other algorithms than the Affymetrix one we have obtained different numerical values representing the same expression values and expression differences upon comparison.

The differential expression E in one of the breast cancer groups compared to the normal population is calculated as follows. Given n average difference values d₁, d₂, . . . , dₙ in the breast cancer population and m average difference values c₁, c₂, . . . , cₘ in the population of normal individuals, it is computed by the equation:

$$E ≡ exp ( \frac{1}{m} {\sum }_{i = 1}^{m} lm ( {c}_{i} ) - \frac{1}{n} {\sum }_{i = 1}^{n} ln ( {d}_{i} ) ) \qquad ( equation 1 )$$

If dⱼ<50 or cᵢ<50 for one or more values of i and j, these particular values cᵢ and/or dⱼ are set to an “artificial” expression value of 50. These particular computation of E allows for a correct comparison to TaqMan results.

A gene is called up-regulated in breast cancer versus normal if E≧minimal change factor given in Table 3 and if the number of absolute calls equal to ‘P’ in the breast cancer population is greater than n/2. The minimal fold change factors in Table 3 are given for those patient populations responding to a given chemotherapy (CR), non responding to a administered chemotherapy (NC) or those tissues without any pathological signs of a tumor (NB). Fold changes greater than 1 refers to an increase in gene expression in the first names tissue sample compared to the second. This regulation factors are mean values and may differ individually, here the combined profiles of all 185 genes listed in Table 1a and 1b in a cluster analysis or a principle component analysis will indicate the classification group for such sample.

According to the above, a gene is called down-regulated in breast cancer versus normal if E≦minimal change factor given in Table 3 and if the number of absolute calls equal to ‘P’ in the breast cancer population is greater than n/2. Values smaller than 1 describe an decreased expression of the given gene.

The minimal fold change factors given in Table 3 indicate also the relative up- and down-regulation of those gene indicative of tumor presence. These genes do show in the comparison of any tumor tissue to the normal healthy counterpart (NT) the highest increase or decrease factors (e.g. SEQ ID: 43, 55, 65, or 162)

The final list of differentially regulated genes consists of all up-regulated and all down-regulated genes in biological samples from individuals with breast cancer versus biological samples from the normal population or of an individual response pattern. Those genes on this list which are interesting for a diagnostic or pharmaceutical application were finally validated by quantitative real time RT-PCR (see Example 1). If a good correlation between the expression values/behavior of a transcript could be observed with both techniques, such a gene is listed in Tables 1 to 5.

### EXAMPLE 4

### Analysis of Differential Gene Expression Patterns Using Support Vector Machines

Support vector machines (SVM) are well suited for two-class or multi-class pattern recognition (Weston and Watkins, 1999 (115); Vapnik, 1995 (116); Vapnik, 1998 (117); Burges, 1998 (118).

For the two-class classification problem, (e.g. tumor tissue vs. non tumor tissue, or therapy response vs. non response) assume that we have a set of samples, i.e., a series of input vectors \vec{xᵢ}εR^{d} (i=1, 2, . . . , m)

with corresponding labels

yᵢε{+1,−1} (i=1, 2, . . . , m).

Here, +1 and −1 indicate the two classes. To classify gene expression patterns of marker genes from Table 1a and 1b or 2 for describing the current tumor status or probable response to a therapeutic agent, the input vector dimension is equal to the number of different oligonucleotide types present on the oligonucleotide array or a subset hereof, and each input vector unit stands for the hybridization value of one specific oligonucleotide type.

The goal is to construct a binary classifier or derive a decision function from the available samples which has a small probability of misclassifying a future sample.

An SVM implements the following idea: it maps the input vectors

\vec{xᵢ}εR^{d}

into a high-dimensional feature space

Φ(\vec{x})εH

and constructs an Optimal Separating Hyperplane (OSH), which maximizes the margin, the distance between the hyperplane and the nearest data points of each class in the space if. By choosing OSH from among the many that can separate the positive from the negative examples in the feature space, SVMs are avoiding the risk of overfitting.

Different mappings construct different SVMs. The mapping Φ:R^{d} [symbol] H

is performed by a kernel function

K(\vec{xᵢ},\vec{xⱼ})

which defines an inner product in the space H.

The decision function implemented by SVM can be written as (Burges, 1998 (118):

$$f ( {x}^{->} ) = sgn ( {\sum }_{i = 1}^{m} {y}_{i} {α}_{i} \cdot K ( {x}^{->} , {{x}^{->}}_{i} ) + b ) \qquad ( equation 2 )$$

where the coefficients αᵢ are obtained by solving the following convex Quadratic Programming (QP) problem:

$$Maximize {\sum }_{i = 1}^{m} {α}_{i} - \frac{1}{2} {\sum }_{i = 1}^{m} {\sum }_{j = 1}^{m} {α}_{i} {α}_{j} \cdot {y}_{i} {y}_{j} \cdot K ( {{x}^{->}}_{i} , {{x}^{->}}_{j} ) \\ subject to 0 \leq {α}_{i} \leq C \\ and {\sum }_{i = 1}^{m} {α}_{i} {y}_{i} = 0 \qquad ( equation 3 )$$

The regularity parameter C (equation 3) controls the trade off between margin and misclassification error. The \vec{x}ⱼ are called Support Vectors only if the corresponding αᵢ>0.

Two of the kernel functions used in the current example:

K(\vec{xᵢ},\vec{xⱼ})=(\vec{xᵢ}·\vec{xⱼ}+1)^{d} (equation 4)

K(\vec{xᵢ},\vec{xⱼ})=e^{(-r|}\vec{x^{ᵢ}}⁻\vec{x^{ⱼ}}^{|}^{²}⁾ (equation 5)

where the first one (equation 4) is called the polynomial kernel function of degree d which will eventually revert to the linear function when d=1, the latter (equation 5) is called the Radial Basic Function (RBF) kernel.

For a given data set, only the kernel function and the regularity parameter C must be selected to specify one SVM. An SVM has many attractive features. For instance, the solution of the QP problem is globally optimised while with neural networks the gradient based training algorithms only guarantee finding a local minima. In addition, SVM can handle large feature spaces, can effectively avoid overfitting (see above) by controlling the margin, can automatically identify a small subset made up of informative points, i.e., the Support Vectors, etc.

The classification of biological sample and thereby the identification of an neoplastic lesion as well as the response of such lesion to therapeutic agents based on gene expression data is a multi-class classification problem. The class number k is equal to the number tumor subclasses (e.g. histological features, TNM stage, grade, hormonal status) and is equal to response subgroupe to a certain therapeutic agent (e.g. pathologicaly confirmed complete remission, good remission, partial remission, or no remission, as well as progressive disease) which shall be predicted, i.e., which are present in the training data set. Due to the limited number of different classes in the present sample set, we decided to handle the multi-class classification by reducing the multi-classification to a series of binary classifications. For a k-class classification, k SVMs are constructed. The ith SVM will be trained with all of the samples in the ith class with positive labels and all other samples with negative labels. Finally an unknown sample is classified into the class that corresponds to the SVM with the highest output value. This method is used to construct a prediction/classification system for gene expression patterns of differentially expressed marker genes as given in Table 1a and 1b and 2.

Each data point generated by a microarray hybridization experiment or by real time RT-PCR (cf. example 1 and 2) corresponds to and is determined by the number of mRNA copies present in the analysed sample, i.e., from an experiment with n oligonucleotide types on a polynucleotide array, a series of n expression-level values is obtained. These n values are typically stored in a metrics file which is the result of the analysis of a “cel file” by the Affymetrix® Microarray Suite or software described above. The data from a series of m metrics files (representing m expression analyses) are taken to build an expression matrix, in which each of the m rows consists of an n-element expression vector for a single experiment. In order to normalise the expression values of the m experiments, we define x_{i,j} to be the sum of the logarithms of the expression level α_{i,j} for gene j (whose mRNA hybridizes with the oligonucleotide type j′ present on the microarray, or gives a valid ΔΔC_{T} intensity), normalized so that the expression vector \vec{xᵢ} has the Euclidean length l:

$${x}_{j , i} = \frac{ln ( {a}_{i , j} )}{\sqrt{{\sum }_{k = 1}^{n} {ln ( {a}_{i , k} )}^{2}}} \qquad ( equation 6 )$$

Initial analyses are carried out using a set of 20000-element expression vectors for 150 experiments as described in example 1 and 2 (100 experiments in the training set and 50 in the test set).

Using the knowledge that the 150 experiments represent three different response classes and two different tumor states as well as the information of tumor and non-tumor tissue, we trained the SVMs described above with the training set to recognize those response classes and disease states. The test set was used to assess the prediction accuracy. Here we have preformed crossvalidations utilizing the “leave one out” method and for more stringent testing a four to five fold validation (leave 25% out) with n iterations (n>100).

In such crossvalidations and classification experiments the predictive power of a subset of marker genes chosen from Table 1a and 1b (e.g. SEQ ID: 27, 38, 55, 81, 97, 98) has been tested. The average cross validation error rate was 8.333% with affinity levels as follows:

| Tissue sample | True response | Predicted CR | Predicted NC |
|---|---|---|---|
| Sample_1 | CR | 0.9141 | −0.9141 |
| Sample_2 | CR | 1.281 | −1.281 |
| Sample_3 | CR | 1.149 | −1.149 |
| Sample_4 | CR | 0.3987 | −0.3987 |
| Sample_5 | CR | 0.2182 | −0.2182 |
| Sample_6 | CR | 0.7127 | −0.7127 |
| Sample_7 | NC | −1.124 | 1.124 |
| Sample_8 | NC | −1.492 | 1.492 |
| Sample_9 | NC | −1.896 | 1.896 |
| Sample_10 | NC | 0.475 | −0.475 |
| Sample_11 | NC | −1.962 | 1.962 |
| Sample_12 | NC | −0.7557 | 0.7557 |

The misclassification of one sample can be compensated by addition of more marker genes from Table 1a and 1b. These data show the minimal number of marker genes that could be combined for a predictive assay or kit.

### EXAMPLE 5

In order to optimize prediction of non responding tumor samples one may use this class from the trainings cohort and run multiple statistical tests, suitable for group comparison such as t-test or Wilcoxon. As listed in Table 6 one can identify such genes with a differential expression in the non responding tumor tissue and a significance level (p-value) below 0.05. In Table 6 20 genes are selected fulfilling the criterion of low p-value and high expressional fold change between the two classes.

One may combine the gene list selected as most preferred given in Table 2 with those genes from Table 1b and performe classification experiments for any so far unclassified sample and predict response to chemotherapy.

While as those algorithms described in Example 4 can be implemented in a certain kernel to classify samples according to their specific gene expression into two classes another approach can be taken to predict class membership by implementation of a k-NN classification. The method of k-Nearest Neighbors (k-NN), proposed by T. M. Cover and P. E. Hart, an important approach to nonparametric classification, is quite easy and efficient. Partly because of its perfect mathematical theory, NN method develops into several variations. As we know, if we have infinitely many sample points, then the density estimates converge to the actual density function. The classifier becomes the Bayesian classifier if the large-scale sample is provided. But in practice, given a small sample, the Bayesian classifier usually fails in the estimation of the Bayes error especially in a high-dimensional space, which is called the disaster of dimension. Therefore, the method of k-NN has a great pity that the sample space must be large enough.

In k-nearest-neighbor classification, the training data set is used to classify each member of a “target” data set. The structure of the data is that there is a classification (categorical) variable of interest (e.g. “responder” (CR) or “non-responder” (NC)), and a number of additional predictor variables (gene expression values). Generally speaking, the algorithm is as follows:
1. For each sample in the data set to be classified, locate the k nearest neighbors of the training data set. A Euclidean Distance measure can be used to calculate how close each member of the training set is to the target sample that is being examined.
2. Examine the k nearest neighbors—which classification do most of them belong to? Assign this category to the sample being examined.
3. Repeat this procedure for the remaining samples in the target set.

Of course the computing time goes up as k goes up, but the advantage is that higher values of k provide smoothing that reduces vulnerability to noise in the training data. In practical applications, typically, k is in units or tens rather than in hundreds or thousands.

The “nearest neighbors” are determined if given the considered the vector and the distance measurement. Given a training set of expression values for a certain number of samples T={(x₁, y1), (x2, y2), . . . , (xm, ym)}, to determine the class of the input vector x.

The most special case is the k-NN method, while k=1, which just searches the one nearest neighbor:

j=arg min//x−xi//

then, (x, yj) is the solution.

For estimation on the error rate of this classification the following considerations could be made:

A training set T={(x1, y1), (x2, y2), . . . , (xm, ym)} is called (k, d %)-stable if the error rate of k-NN method is d %, where d % is the empirical error rate from independent experiments. If the clustering of data are quite distinct (the class distance is the crucial standard of classification), then the k must be small. The key idea is we prefer the least k in the case that d % is bigger the threshold value.

The k-NN method gathers the nearest k neighbors and let them vote—the class of most neighbors wins. Theoretically, the more neighbors we consider, the smaller error rate it takes place. The general case is a little more complex. But by imagination, it is true to be the more

k the lower upper bound asymptotic to PBayes(e) if N is fixed.

One can use such algorithm to classify and cross validate a given cohort of samples based on the genes presented by this invention in Tables 1a and 1b. Most preferably the classification shall be performed based on the expression levels of the genes presented in Table 1b in combination with the genes from Table 2. With k=3 and >100 iteration one can get classifications as depicted below for a cross-validation experiment with the three classes “normal breast tissue” (not affected by cancer), non responding tumor (NC), and responding tumor (CR). Affinities ranging from −1 to 1 for a given class.

| | | Predicted | | | |
|---|---|---|---|---|---|
| Tissue | True | normal | Predicted- | Predicted- | |
| sample | response | breast | NC | CR | Remarks |
| “normal” | | 1 | −0.5 | −0.5 | |
| tissue | | | | | |
| Sample_1 | CR | −0.4994 | −0.5 | 0.9994 | |
| Sample_2 | CR | −0.4988 | −0.5 | 0.9988 | |
| Sample_3 | CR | −0.4988 | −0.5 | 0.9988 | |
| Sample_4 | CR | −0.5 | −0.5 | 1 | |
| Sample_5 | CR | −0.4988 | −0.5 | 0.9988 | |
| Sample_6 | CR | −0.5 | −0.5 | 1 | |
| Sample_7 | CR | −0.5 | −0.4988 | 0.9988 | |
| Sample_8 | CR | −0.4883 | −0.4649 | 0.9532 | |
| Sample_9 | NC | −0.497 | 0.997 | −0.5 | |
| Sample_10 | NC | −0.4969 | 0.9969 | −0.5 | |
| Sample_11 | NC | −0.4975 | 0.9975 | −0.5 | |
| Sample_12 | NC | −0.4982 | 0.9982 | −0.5 | |
| Sample_13 | NC | 1 | −0.5 | −0.5 | low tumor |
| | | | | | % |
| Sample_14 | NC | −0.5 | −0.4988 | 0.9988 | false |
| Sample_15 | NC | −0.4976 | 0.9976 | −0.5 | |
| Sample_16 | NC | −0.4976 | 0.9976 | −0.5 | |

The misclassification of one sample can be compensated by addition of more marker genes from Table 1a. These data show the minimal number of marker genes that could be combined for a predictive assay or kit.

### EXAMPLE 6

In order to get the most accurate prediction for response to chemotherapy based on the expression levels of genes listed in Tables 1a and Table 1b. One can implement a step wise classification model identifying first those individuals (tumor tissues) with the highest affinity (e.g. by k-NN classification) to the class of responding tumors (CR). If an so far unclassified tumor sample did not belong to the class of CR on may performe a second classification step for this sample using the expression levels of the genes from Table 1a (e.g. SEQ ID Nos: 2, 8, 9, 21, 24, 35, 53, 54, 57, 64, 80, 87, 89, 95, 97, 118 and 146) which will give in a k-NN classification a better separation of the non responding tumors from those which will respond partially. For this second classification step only the predefined classes NC and PR should be utilized.

### REFERENCES

### Patents Cited

U.S. Pat. No. 4,843,155 Chomczynski, P.
U.S. Pat. No. 5,262,31 Liang, P., and Pardee, A. B., 1993
U.S. Pat. No. 4,683,202 Mullis, K. B., 1987
U.S. Pat. No. 5,593,839
U.S. Pat. No. 5,578,832
U.S. Pat. No. 5,556,752
U.S. Pat. No. 5,631,734
U.S. Pat. No. 5,599,695
U.S. Pat. No. 4,683,195
U.S. Pat. No. 5,498,531
U.S. Pat. No. 5,714,331
U.S. Pat. No. 5,641,673 Haseloff et al.,
U.S. Pat. No. 5,223,409 Lander, E.,
U.S. Pat. No. 5,976,813 Beutel et al.
U.S. Pat. No. 5,283,317
U.S. Pat. No. 6,203,987
WO 97/29212
WO 97/27317
WO 95/22058
WO 99/12826
WO 97/02357
WO 94/13804
WO 94/10300
EP 0 785 280
EP 0 799 897
EP 0 728 520
EP 0 721 016
EP 0 321 201
GB2188638B

### OTHER REFERENCES CITED

(1) Publications cited: WHO. International Classification of Diseases, 10^{th} edition (ICD-10). WHO
(2) Sabin, L. H., Wittekind, C. (eds): TNM Classification of Malignant Tumors. Wiley, New York, 1997
(3) Sorlie et al., Proc Natl Acad Sci USA. 2001 Sep. 11; 98(19):10869-74 (3);
(4) van 't Veer et al., Nature. 2002 Jan. 31; 415(6871):530-6. (4).
(5) Perez, E. A.: Current Management of Metastatic Breast Cancer. Semin. Oncol., 1999; 26 (Suppl. 12): 1-10
(6) Sambrook et al., MOLECULAR CLONING: A LABORATORY MANUAL, 2d ed., 1989
(7) Ausubel et al., CURRENT PROTOCOLS IN MOLECULAR BIOLOGY, John Wiley & Sons, New York, N.Y., 1989.
(8) Tedder, T. F. et al., Proc. Natl. Acad. Sci. U.S.A. 85:208-212, 1988
(9) Hedrick, S. M. et al., Nature 308:149-153, 1984
(10) Lee, S. W. et al., Proc. Natl. Acad. Sci. U.S.A. 88:4225, 1984
(11) Sarkar, PCR Methods Applic. 2, 318-322, 1993
(12) Triglia et al., Nucleic Acids Res. 16, 81-86, 1988
(13) Lagerstrom et al., PCR Methods Applic. 1, 111-119, 1991
(14) Copeland & Jenkins, Trends in Genetics 7: 113-118, 1991
(15) Cohen, et al., Nature 366: 698-701, 1993
(16) Bonner et al., J. Mol. Biol. 81, 123 1973
(17) Bolton and McCarthy, Proc. Natl. Acad. Sci. U.S.A. 48, 1390 1962
(19) Altschul et al., Bull. Math. Bio. 48:603, 1986,
(20) Henikoff & Henikoff, Proc. Natl. Acad. Sci. USA 89:10915, 1992
(21) Pearson & Lipman, Proc. Nat'l Acad. Sci. USA 85:2444, 1988
(22) Pearson et al., Meth. Enzymol. 183:63, 1990
(23) Needleman & Wunsch, J. Mol. Biol. 48:444, 1970
(24) Sellers, SIAM J. Appl. Math.Xno:787, 1974
(25) Takamatsu, EMBO J. 6, 307-311, 1987
(26) Coruzzi et al., EMBO J. 3, 1671-1680, 1984
(27) Broglie et al., Science 224, 838-843, 1984
(28) Winter et al., Results Probl. Cell Differ. 17, 85-105, 1991
(29) Engelhard et al., Proc. Nat. Acad. Sci. 91, 3224-3227, 1994
(30) Logan & Shenk, Proc. Natl. Acad. Sci. 81, 3655-3659, 1984
(31) Scharf et al., Results Probl. Cell Differ. 20, 125-162, 1994
(32) Freshney R. I., ed., ANIMAL CELL CULTURE, 1986
(33) Wigler et al., Cell 11, 223-232, 1977
(34) Lowy et al., Cell 22, 817-823, 1980
(35) Wigler et al., Proc. Natl. Acad. Sci. 77, 3567-3570, 1980
(36) Colbere-Garapin et al., J. Mol. Biol. 150, 114, 1981
(37) Hartman & Mulligan, Proc. Natl. Acad. Sci. 85, 8047-8051, 1988
(38) Rhodes et al., Methods Mol. Biol. 55, 121-131, 1995
(39) Hampton et al., SEROLOGICAL METHODS: A LABORATORY MANUAL, APS Press, St. Paul, Minn., 1990
(40) Maddox et al., J. Exp. Med. 158, 1211-1216, 1983
(41) Porath et al., Prot. Exp. Purif. 3, Xno3-281, 1992
(42) Kroll et al., DNA Cell Biol. 12, 441-453, 1993
(43) Caruthers et al., Nucl. Acids Res. Symp. Ser. 215-223, 1980
(44) Horn et al. Nucl. Acids Res. Symp. Ser. 225-232, 1980
(45) Merrifield, J. Am. Chem. Soc. 85, 2149-2154, 1963
(46) Roberge et al., Science Xno9, 202-204, 1995
(47) Creighton, PROTEINS: STRUCTURES AND MOLECULAR PRINCIPLES, WH and Co., New York, N.Y., 198
(48) Cronin et al., Human Mutation 7:244, 1996
(49) Landegran et al., Science 241:1077-1080, 1988
(50) Nakazawa et al., PNAS 91:360-364, 1994
(51) Abravaya et al., Nuc Acid Res 23:675-682, 1995
(52) Guatelli, J. C. et al., Proc. Natl. Acad. Sci. USA 87:1874-1878, 1990
(53) Kwoh, D. Y. et al., Proc. Natl. Acad. Sci. USA 86:1173-1177, 1989
(54) Lizardi, P. M. et al., Bio/Technology 6:1197, 1988
(55) Brown, Meth. Mol. Biol. 20, 18, 1994
(56) Sonveaux, Meth. Mol. Biol. Xno, 1-72, 1994
(57) Uhlmann et al., Chem. Rev. 90, 543-583, 1990
(58) Gee et al., in Huber & Carr, MOLECULAR AND IMMUNOLOGIC APPROACHES, Publishing Co., Mt. Kisco, N.Y., 1994
(59) Agrawal et al., Trends Biotechnol. 10, 152-158, 1992
(60) Uhlmann et al., Tetrahedron. Lett. 215, 3539-3542, 1987
(61) Cech, Science 236, 1532-1539, 1987
(62) Cech, Ann. Rev. Biochem. 59, 543-568, 1990
(63) Couture & Stinchcomb, Trends Genet. 12, 510-515, 1996
(64) Haseloff et al. Nature 334, 585-591, 1988
(65) Kohler et al., Nature 256, 495-497, 1985
(66) Kozbor et al., J. Immunol. Methods 81, 3142, 1985
(67) Cote et al., Proc. Natl. Acad. Sci. 80, 20Xno-2030, 1983
(68) Cole et al., Mol. Cell. Biol. 62, 109-120, 1984
(69) Morrison et al., Proc. Natl. Acad. Sci. 81, 6851-6855, 1984
(70) Neuberger et al., Nature 312, 604-608, 1984
(71) Takeda et al., Nature 314, 452-454, 1985
(72) Burton, Proc. Natl. Acad. Sci. 88, 11120-11123, 1991
(73) Thirion et al., Eur. J. Cancer Prev. 5, 507-11, 1996
(74) Coloma & Morrison, Nat. Biotechnol. 15, 159-63, 1997
(75) Mallender & Voss, J. Biol. Chem. Xno9, 199-206, 1994
(76) Verhaar et al., Int. J. Cancer 61, 497-501, 1995
(77) Nicholls et al., J. Immunol. Meth. 165, 81-91, 1993
(78) Orlandi et al., Proc. Natl. Acad. Sci. 86, 3833-3837, 1989
(79) Winter et al., Nature 349, 293-299, 1991
(80) Lam, Anticancer Drug Des. 12, 145, 1997
(81) DeWitt et al., Proc. Natl. Acad. Sci. U.S.A. 90, 6909, 1993
(82) Erb et al. Proc. Natl. Acad. Sci. U.S.A. 91, 11422, 1994
(83) Zuckermann et al., J. Med. Chem. 37, Xno78, 1994
(84) Cho et al., Science Xnol, 1303, 1993
(85) Carell et al., Angew. Chem. Int. Ed. Engl. 33, 2059 & 2061, 1994
(86) Gallop et al., J. Med. Chem. 37, 1233, 1994
(87) Houghten, BioTechniques 13, 412-421, 1992
(88) Lam, Nature 354, 8284, 1991
(89) Fodor, Nature 364, 555-556, 1993
(90) Cull et al., Proc. Natl. Acad. Sci. U.S.A. 89, 1865-1869, 1992
(91) Scott & Smith, Science 249, 386-390, 1990
(92) Devlin, Science 249, 404-406, 1990
(93) Cwirla et al., Proc. Natl. Acad. Sci. 97, 6378-6382, 1990
(94) Felici, J. Mol. Biol. 222, 301-310, 1991
(95) Jayawickreme et al., Proc. Natl. Acad. Sci. U.S.A. 19, 1614-1618, 1994
(96) Chelsky, Strategies for Screening Combinatorial Libraries 1995
(97) Salmon et al., Molecular Diversity 2, 57-63, 1996
(98) McConnell et al., Science 257, 1906-1912, 1992
(99) Sjolander & Urbaniczky, Anal. Chem. 63, 2338-2345, 1991
(100) Szabo et al., Curr. Opin. Struct. Biol. 5, 699-705, 1995
(101) Zervos et al., Cell 72, 223-232, 1993
(102) Madura et al., J. Biol. Chem. Xno8, 12046-12054, 1993
(103) Bartel et al., BioTechniques 14, 920-924, 1993
(104) Iwabuchi et al., Oncogene 8, 1693-1696, 1993
(105) Findeis et al. Trends in Biotechnol. 11, 202-205, 1993
(106) Chiou et al., GENE THERAPEUTICS: METHODS AND APPLICATIONS OF DIRECT GENE TRANSFER J. A. Wolff, ed., 1994
(107) Wu & Wu, J. Biol. Chem. Xno3, 621-24, 1988
(108) Wu et al., J. Biol. Chem. Xno9, 54246, 1994
(109) Zenke et al., Proc. Natl. Acad. Sci. U.S.A. 87, 3655-59, 1990
(110) Wu et al., J. Biol. Chem. Xno6, 33842, 1991
(111) REMINGTON'S PHARMACEUTICAL SCIENCES Maack Publishing Co., Easton, Pa.
(112) Hille, Excitable Membranes, Sunderland, M A, Sinauer Associates, Inc.
(113) Van Heeke & Schuster, J. Biol. Chem. 264, 5503-5509, 1989
(114) Grant et al., Methods Enzymol. 153, 516-544, 1987
(115) Weston and Watkins, Proceedings of the Seventh European Symposium On Artificial Neural Networks, 1999
(116) Vapnik, The Nature of Statistical Learning Theory, 1995, Springer, New York
(117) Vapnik, Statistical Learning Theory, 1998, Wiley, New York
(118) Burges, Data Mining and Knowledge Discovery, 2(2):955-974, 1998

TABLE 1a
List of 165 genes which are differentially expressed in responders compared to
non-responders or normal healthy tissue. Reference is given to the SEQ ID NOs of the
sequence listing.

| SEQ ID NO: | SEQ ID NO: | | Ref. | | |
|---|---|---|---|---|---|
| (DNA | (Protein | | Sequences | | |
| Sequence) | Sequence) | Gene_Symbol | [A] | Gene_ID | Locus_Link_ID |
| 1 | 166 | CTSB | NM_001908 | 4503138 | 1508 |
| 2 | 167 | SSR1 | NM_003144 | 14781630 | 6745 |
| 3 | 168 | STX8 | NM_002803 | 4506208 | 5701 |
| 4 | 169 | KPNA2 | NM_002266 | 4504896 | 3838 |
| 5 | 170 | CSE1L | NM_001316 | 18591914 | 1434 |
| 6 | 171 | RHEB2 | NM_005614 | 18600748 | 6009 |
| 7 | 172 | DKC1 | NM_001363 | 15011921 | 1736 |
| 8 | 173 | IGFBP4 | NM_001552 | 10835020 | 3487 |
| 9 | 174 | SMC1L1 | NM_006306 | — | 8243 |
| 10 | 175 | PWP1 | NM_007062 | 5902033 | 11137 |
| 11 | 176 | HDAC2 | NM_001527 | 4557640 | 3066 |
| 12 | 177 | PRKAB1 | NM_006253 | 18602783 | 5564 |
| 13 | 178 | IMPDH2 | NM_000884 | 4504688 | 3615 |
| 14 | 179 | UBE2A | NM_003336 | 4507768 | 7319 |
| 15 | 180 | YR-29 | NM_014886 | 7662676 | 10412 |
| 16 | 181 | MUF1 | NM_006369 | 5453747 | 10489 |
| 17 | 182 | MYO10 | NM_012334 | 11037056 | 4651 |
| 18 | 183 | EGFR | NM_005228 | 4885198 | 1956 |
| 19 | 184 | IFRD1 | NM_001550 | 4504606 | 3475 |
| 20 | 185 | CD2BP2 | NM_006110 | 5174408 | 10421 |
| 21 | 186 | ARL3 | NM_004311 | 4757773 | 403 |
| 22 | 187 | CCNB2 | NM_004701 | 10938017 | 9133 |
| 23 | 188 | FMOD | NM_002023 | 18548671 | 2331 |
| 24 | 189 | SLC7A8 | NM_012244 | 14751202 | 23428 |
| 25 | 190 | E2-EPF | NM_014501 | 7657045 | 27338 |
| 26 | 191 | AGT | NM_000029 | 4557286 | 183 |
| 27 | 192 | FHL2 | NM_001450 | 4503722 | 2274 |
| 28 | 193 | LDLC | NM_007357 | 6678675 | 22796 |
| 29 | 194 | MGC16824 | NM_020314 | 10092674 | 57020 |
| 30 | 195 | UGDH | NM_003359 | 4507812 | 7358 |
| 31 | 196 | MAD2L1 | NM_002358 | 6466452 | 4085 |
| 32 | 197 | DDB2 | NM_000107 | 4557514 | 1643 |
| 33 | 198 | OS4 | NM_005730 | 5031964 | 10106 |
| 34 | 199 | BCL2 | NM_000633 | 13646672 | 596 |
| 35 | 200 | SEMA3C | NM_006379 | 5454047 | 10512 |
| 36 | 201 | DTR | NM_001945 | 4503412 | 1839 |
| 37 | 202 | GARP | NM_005512 | 5031706 | 2615 |
| 38 | 203 | ACK1 | NM_005781 | 8922074 | 10188 |
| 39 | 204 | EDG2 | NM_001401 | 16950637 | 1902 |
| 40 | 205 | RARRES3 | NM_004585 | 8051633 | 5920 |
| 41 | 206 | CCNH | NM_001239 | 17738313 | 902 |
| 42 | 207 | PREP | NM_002726 | 4506042 | 5550 |
| 43 | 208 | COL11A1 | NM_001854 | 18548530 | 1301 |
| 44 | 209 | GALC | NM_000153 | 4557612 | 2581 |
| 45 | 210 | HMGCS2 | NM_005518 | 5031750 | 3158 |
| 46 | 211 | ZNF274 | NM_016324 | 7706506 | 10782 |
| 47 | 212 | TFF1 | NM_003225 | 4507450 | 1031 |
| 48 | 213 | RAD51 | NM_002875 | 4506388 | 5888 |
| 49 | 214 | ASNS | NM_001673 | 4502258 | 440 |
| 50 | 215 | PCMT1 | NM_005389 | 4885538 | 5110 |
| 51 | 216 | ESR1 | NM_000125 | 4503602 | 2099 |
| 52 | 217 | ACAT1 | NM_000019 | 4557236 | 38 |
| 53 | 218 | XPA | NM_000380 | 4507936 | 7507 |
| 54 | 219 | LAF4 | NM_002285 | 4504938 | 3899 |
| 55 | 220 | COL10A1 | NM_000493 | 18105031 | 1300 |
| 56 | 221 | KIAA1041 | NM_014947 | 15299048 | 22887 |
| 57 | 222 | PLA2G7 | NM_005084 | 4826883 | 7941 |
| 58 | 223 | GRP | NM_002091 | 4504158 | 2922 |
| 59 | 224 | CYP2B6 | NM_000767 | 14550410 | 1555 |
| 60 | 225 | CHAD | NM_001267 | 4502798 | 1101 |
| 61 | 226 | GALNT10 | NM_017540 | 9055207 | 55568 |
| 62 | 227 | GADD45B | NM_015675 | 9945331 | 4616 |
| 63 | 228 | WBSCR20 | NM_017528 | 8923713 | 114049 |
| 64 | 229 | BTBD2 | NM_017797 | 8923361 | 55643 |
| 65 | 230 | PGR | NM_000926 | 4505766 | 5241 |
| 66 | 231 | TBPL1 | NM_004865 | 4759233 | 9519 |
| 67 | 232 | C4B | NM_000592 | 14577918 | 721 |
| 68 | 233 | CCNG1 | NM_004060 | — | 900 |
| 69 | 234 | PDHB | NM_000925 | 4505686 | 5162 |
| 70 | 235 | HNRPDL | NM_005463 | 14110410 | 9987 |
| 71 | 236 | TAF11 | NM_005643 | 5032150 | 6882 |
| 72 | 237 | AMACR | NM_014324 | 14725899 | 23600 |
| 73 | 238 | EMD | NM_000117 | 4557552 | 2010 |
| 74 | 239 | NR2F1 | NM_005654 | 5032172 | 7025 |
| 75 | 240 | HSF2 | NM_004506 | 6806888 | 3298 |
| 76 | 241 | SPG4 | NM_014946 | — | 6683 |
| 77 | 242 | TRIP11 | NM_004239 | 10863904 | 9321 |
| 78 | 243 | OCLN | NM_002538 | 9257230 | 4950 |
| 79 | 244 | CACNA1D | NM_000720 | — | 776 |
| 80 | 245 | CYP2B7 | NR_001278 | 14550410 | 1556 |
| 81 | 246 | FHL1 | NM_001449 | 4503720 | 2273 |
| 82 | 247 | MSX2 | NM_002449 | 18560141 | 4488 |
| 83 | 248 | PAI-RBP1 | NM_015640 | 7661625 | 26135 |
| 84 | 249 | CLDN14 | NM_012130 | 18593128 | 23562 |
| 85 | 250 | ITPK1 | NM_014216 | 18583687 | 3705 |
| 86 | 251 | ERBB2 | NM_004448 | 4758297 | 2064 |
| 87 | 252 | TP53 | NM_000546 | 8400737 | 7157 |
| 88 | 253 | HSPA2 | NM_021979 | 13676856 | 3306 |
| 89 | 254 | LIG1 | NM_015541 | 18554950 | 26018 |
| 90 | 255 | GSS | NM_000178 | 4504168 | 2937 |
| 91 | 256 | PRO1843 | NM_018507 | 8924082 | 55378 |
| 92 | 257 | MKI67 | NM_002417 | 4505188 | 4288 |
| 93 | 258 | BIK | NM_001197 | 7262371 | 638 |
| 94 | 259 | KIAA0225 | D86978 | 18566873 | 23165 |
| 95 | 260 | TNRC15 | AB014542 | 18550089 | 26058 |
| 96 | 261 | SFRS5 | NM_006925 | 5902077 | 6430 |
| 97 | 262 | RPL17 | NM_000985 | 14591906 | 6139 |
| 98 | 263 | GNG12 | NM_018841 | — | 55970 |
| 99 | 264 | LAP1B | NM_015602 | 17488747 | 26092 |
| 100 | 265 | LOC253782 | AL080192 | — | 253782 |
| 101 | 266 | COL5A1 | NM_000093 | 18571690 | 1289 |
| 102 | 267 | CXCL13 | NM_006419 | 5453576 | 10563 |
| 103 | 268 | TTS-2.2 | AF055000 | 3231586CB1 | 57104 |
| 104 | 269 | KIAA0056 | D29954 | 18578675 | 23310 |
| 105 | 270 | FLJ22642 | AI700633 | — | — |
| 106 | 271 | LOC113146 | W28438 | 15300131 | 113146 |
| 107 | 272 | GPR126 | NM_020455 | 18562351 | 57211 |
| 108 | 273 | PMSCL1 | NM_005033 | 4826921 | 5393 |
| 109 | 274 | KIAA0418 | NM_014631 | 7662103 | — |
| 110 | 275 | SULF1 | NM_015170 | 18571189 | 23213 |
| 111 | 276 | KIAA0673 | NM_015102 | 14720169 | 261734 |
| 112 | 277 | FLJ10803 | NM_018224 | — | 55744 |
| 113 | 278 | DKFZp586M0723 | AL050227 | — | — |
| 114 | 279 | C4A | NM_007293 | 14577920 | 720 |
| 115 | 280 | ZAP3 | L40403 | 18597333 | 56252 |
| 116 | 281 | NEK9 | NM_033116 | 14916458 | 91754 |
| 117 | 282 | FLJ13125 | AK023187 | 14726621 | — |
| 118 | 283 | FMO5 | NM_001461 | 4503760 | 2330 |
| 119 | 284 | COMP | NM_000095 | 4557482 | 1311 |
| 120 | 285 | CSPG2 | NM_004385 | 4758081 | 1462 |
| 121 | 286 | LOC151996 | AA418080 | 18554956 | — |
| 122 | 287 | TFAP2B | NM_003221 | 4507442 | 7021 |
| 123 | 288 | OR7E38P | AF065854 | 18544324 | 10821 |
| 124 | 289 | RAB31 | NM_006868 | 5803130 | 11031 |
| 125 | 290 | HSPC126 | NM_014166 | 14759175 | 29079 |
| 126 | 291 | UMP-CMPK | NM_016308 | 7706496 | 51727 |
| 127 | 292 | FLJ22195 | NM_022758 | 12232426 | 64771 |
| 128 | 293 | DCTN4 | NM_016221 | 14733974 | 51164 |
| 129 | 294 | FLJ20273 | NM_019027 | 9506670 | 54502 |
| 130 | 295 | KIF4A | NM_012310 | 14765683 | 24137 |
| 131 | 296 | THTP | NM_024328 | 13236576 | 79178 |
| 132 | 297 | PLSCR4 | NM_020353 | 9966818 | 57088 |
| 133 | 298 | FLJ11323 | NM_018390 | 8922994 | 55344 |
| 134 | 299 | MGC11242 | NM_024320 | 13236560 | 79170 |
| 135 | 300 | CEGP1 | NM_020974 | 10190747 | 57758 |
| 136 | 301 | SRR | NM_021947 | 8922495 | 63826 |
| 137 | 302 | HSPC177 | NM_015961 | 7705488 | 51510 |
| 138 | 303 | MGC3103 | NM_024036 | 13128987 | 78999 |
| 139 | 304 | FLJ20641 | NM_017915 | 8923595 | 55010 |
| 140 | 305 | FLJ13646 | NM_024584 | 13375767 | 79635 |
| 141 | 306 | KCNK15 | NM_022358 | 16507967 | 60598 |
| 142 | 307 | RNASEL | NM_021133 | 10863928 | 6041 |
| 143 | 308 | CRSP6 | NM_004268 | 18577903 | 9440 |
| 144 | 309 | COL5A2 | NM_000393 | 16554580 | 1290 |
| 145 | 310 | LOC51218 | NM_016417 | 9994192 | 51218 |
| 146 | 311 | APBB2 | NM_173075 | 18557629 | 323 |
| 147 | 312 | yy15c12.s1 | N31716 | — | — |
| 148 | 313 | AD037 | NM_032023 | 14042936 | 83937 |
| 149 | 314 | FLJ20477 | AA203365 | 8923441 | — |
| 150 | 315 | MARKL1 | NM_031417 | 13899224 | 57787 |
| 151 | 316 | LUM | NM_002345 | 4505046 | 4060 |
| 152 | 317 | COL3A1 | NM_000090 | 15149480 | 1281 |
| 153 | 318 | COL1A1 | NM_000088 | 18587373 | 1277 |
| 154 | 319 | BF | NM_001710 | 14550403 | 629 |
| 155 | 320 | ADAM12 | NM_003474 | 13259517 | 8038 |
| 156 | 321 | LOXL1 | NM_005576 | 5031882 | 4016 |
| 157 | 322 | CEACAM6 | NM_002483 | 4505340 | 4680 |
| 158 | 323 | MMP11 | NM_005940 | 13027795 | 4320 |
| 159 | 324 | MMP1 | NM_002421 | 13027798 | 4312 |
| 160 | 325 | MMP13 | NM_002427 | 13027796 | 4322 |
| 161 | 326 | SERPINH1 | NM_001235 | 4757923 | 872 |
| 162 | 327 | PITX1 | NM_002653 | 4505824 | 5307 |
| 163 | 328 | RAD52 | NM_015419 | 18390318 | 25878 |
| 164 | 329 | INHBA | NM_002192 | 4504698 | 3624 |
| 165 | 330 | CSPG2 | NM_004385 | 4758081 | 1462 |

TABLE 1b
List of 20 genes which are differentially expressed in non-responding tumors
compared to tumors with at least a minor therapy assosiated regression or normal
healthy tissue. Reference is given to the SEQ ID NOs of the sequence listing.

| SEQ ID NO: | SEQ ID NO: | | Ref. | | |
|---|---|---|---|---|---|
| (DNA | (Protein | | Sequences | | |
| Sequence) | Sequence) | Gene_Symbol | [A] | UniGene_ID | Locus_Link_ID |
| 472 | 492 | PRG1 | NM_002727 | 1908 | 5552 |
| 473 | 493 | GBP1 | NM_002053 | 62661 | 2633 |
| 474 | 494 | ALEX2 | NM_014782 | 48924 | 9823 |
| 475 | 495 | CD53 | NM_000560 | 82212 | 963 |
| 476 | 496 | VCAM1 | NM_001078 | 109225 | 7412 |
| 477 | 497 | MAPT | NM_005910 | 101174 | 4137 |
| 478 | 498 | EGR2 | NM_000399 | 1395 | 1959 |
| 479 | 499 | TDO2 | NM_005651 | 183671 | 6999 |
| 480 | 500 | ADAMDEC1 | NM_014479 | 145296 | 27299 |
| 481 | 501 | TFEC | NM_012252 | 113274 | 22797 |
| 482 | 502 | BTF3 | NM_001207 | 101025 | 689 |
| 483 | 503 | FLNB | NM_001457 | 81008 | 2317 |
| 484 | 504 | TFRC | NM_003234 | 77356 | 7037 |
| 485 | 505 | EIF4B | NM_001417 | 93379 | 1975 |
| 486 | 506 | MAPK3 | — | 861 | 5595 |
| 487 | 507 | LOC161291 | — | 85335 | 161291 |
| 488 | 508 | SLC1A1 | NM_004170 | 91139 | 6505 |
| 489 | 509 | MST4 | NM_016542 | 23643 | 51765 |
| 490 | 510 | BLAME | NM_014036 | 20450 | 56833 |
| 491 | 511 | NME7 | NM_013330 | 274479 | 29922 |

TABLE 2
List of 47 preferred genes which differentially expressed in responders compared to
non responders or normal healthy tissue. Listed genes are preferred genes, e.g., for
use in the assessment whether or not a subject is expected to respond or not to
respond to a given mode of treatment.

| SEQ ID NO: | SEQ ID NO: | | Ref. | | |
|---|---|---|---|---|---|
| (DNA | (Protein | | Sequences | | |
| Sequence) | Sequence) | Gene Symbol | [A] | Gene_ID | Locus_Link_ID |
| 4 | 169 | KPNA2 | NM_002266 | 4504896 | 3838 |
| 5 | 170 | CSE1L | NM_001316 | 18591914 | 1434 |
| 6 | 171 | RHEB2 | NM_005614 | 18600748 | 6009 |
| 7 | 172 | DKC1 | NM_001363 | 15011921 | 1736 |
| 8 | 173 | IGFBP4 | NM_001552 | 10835020 | 3487 |
| 11 | 176 | HDAC2 | NM_001527 | 4557640 | 3066 |
| 12 | 177 | PRKAB1 | NM_006253 | 18602783 | 5564 |
| 13 | 178 | IMPDH2 | NM_000884 | 4504688 | 3615 |
| 15 | 180 | YR-29 | NM_014886 | 7662676 | 10412 |
| 22 | 187 | CCNB2 | NM_004701 | 10938017 | 9133 |
| 23 | 188 | FMOD | NM_002023 | 18548671 | 2331 |
| 24 | 189 | SLC7A8 | NM_012244 | 14751202 | 23428 |
| 25 | 190 | E2-EPF | NM_014501 | 7657045 | 27338 |
| 26 | 191 | AGT | NM_000029 | 4557286 | 183 |
| 27 | 192 | FHL2 | NM_001450 | 4503722 | 2274 |
| 29 | 194 | MGC16824 | NM_020314 | 10092674 | 57020 |
| 31 | 196 | MAD2L1 | NM_002358 | 6466452 | 4085 |
| 32 | 197 | DDB2 | NM_000107 | 4557514 | 1643 |
| 40 | 205 | RARRES3 | NM_004585 | 8051633 | 5920 |
| 43 | 208 | COL11A1 | NM_001854 | 18548530 | 1301 |
| 50 | 215 | PCMT1 | NM_005389 | 4885538 | 5110 |
| 51 | 216 | ESR1 | NM_000125 | 4503602 | 2099 |
| 55 | 220 | COL10A1 | NM_000493 | 18105031 | 1300 |
| 58 | 223 | GRP | NM_002091 | 4504158 | 2922 |
| 61 | 226 | GALNT10 | NM_017540 | 9055207 | 55568 |
| 65 | 230 | PGR | NM_000926 | 4505766 | 5241 |
| 68 | 233 | CCNG1 | NM_004060 | — | 900 |
| 69 | 234 | PDHB | NM_000925 | 4505686 | 5162 |
| 74 | 239 | NR2F1 | NM_005654 | 5032172 | 7025 |
| 81 | 246 | FHL1 | NM_001449 | 4503720 | 2273 |
| 82 | 247 | MSX2 | NM_002449 | 18560141 | 4488 |
| 83 | 248 | PAI-RBP1 | NM_015640 | 7661625 | 26135 |
| 92 | 257 | MKI67 | NM_002417 | 4505188 | 4288 |
| 98 | 263 | GNG12 | NM_018841 | — | 55970 |
| 100 | 265 | LOC253782 | AL080192 | — | 253782 |
| 101 | 266 | COL5A1 | NM_000093 | 18571690 | 1289 |
| 104 | 269 | KIAA0056 | D29954 | 18578675 | 23310 |
| 105 | 270 | FLJ22642 | AI700633 | — | — |
| 106 | 271 | LOC113146 | W28438 | 15300131 | 113146 |
| 108 | 273 | PMSCL1 | NM_005033 | 4826921 | 5393 |
| 113 | 278 | DKFZp586M0723 | AL050227 | — | — |
| 124 | 289 | RAB31 | NM_006868 | 5803130 | 11031 |
| 128 | 293 | DCTN4 | NM_016221 | 14733974 | 51164 |
| 132 | 297 | PLSCR4 | NM_020353 | 9966818 | 57088 |
| 129 | 294 | FLJ20273 | NM_019027 | 9506670 | 54502 |
| 133 | 298 | FLJ11323 | NM_018390 | 8922994 | 55344 |
| 138 | 303 | MGC3103 | NM_024036 | 13128987 | 78999 |

TABLE 3
Relative expression of 165 genes in complete responders as compared to non-
responders and normal tissue. (CR—complete responder to therapy;
NC—no change in tumor state; NT—normal healthy tissue)

| SEQ ID NO: | SEQ ID NO: | | | | |
|---|---|---|---|---|---|
| (DNA | (Protein | | | | |
| Sequence) | Sequence) | Gene_Symbol | CR_vs._NC | CR_vs_NT | NC_vs_NT |
| 1 | 166 | CTSB | 1.69033759 | 2.53990608 | 1.50260284 |
| 2 | 167 | SSR1 | 1.69676002 | 1.56735024 | 0.92373125 |
| 3 | 168 | STX8 | 1.42795315 | 1.65931125 | 1.16202079 |
| 4 | 169 | KPNA2 | 2.10809096 | 2.08540708 | 0.98923961 |
| 5 | 170 | CSE1L | 2.00249838 | 2.79008752 | 1.39330326 |
| 6 | 171 | RHEB2 | 1.84519193 | 1.60184035 | 0.86811584 |
| 7 | 172 | DKC1 | 2.25597289 | 2.3855889 | 1.0574546 |
| 8 | 173 | IGFBP4 | 0.27862606 | 0.38691248 | 1.38864428 |
| 9 | 174 | SMC1L1 | 1.69816116 | 1.71849631 | 1.01197481 |
| 10 | 175 | PWP1 | 0.64477544 | 0.59496475 | 0.92274723 |
| 11 | 176 | HDAC2 | 3.14799689 | 2.11008385 | 0.67029413 |
| 12 | 177 | PRKAB1 | 0.52384682 | 0.56333165 | 1.07537477 |
| 13 | 178 | IMPDH2 | 0.43342682 | 0.53415121 | 1.23239078 |
| 14 | 179 | UBE2A | 1.56667644 | 1.8748269 | 1.19669056 |
| 15 | 180 | YR-29 | 0.51635771 | 0.3928245 | 0.7607604 |
| 16 | 181 | MUF1 | 1.48621121 | 1.67042393 | 1.12394787 |
| 17 | 182 | MYO10 | 2.64854259 | 1.9657171 | 0.74218822 |
| 18 | 183 | EGFR | 1.84523855 | 0.3988927 | 0.21617406 |
| 19 | 184 | IFRD1 | 2.34518159 | 0.67841153 | 0.28927889 |
| 20 | 185 | CD2BP2 | 0.40973605 | 0.74398402 | 1.81576414 |
| 21 | 186 | ARL3 | 0.46877208 | 0.81409499 | 1.73665419 |
| 22 | 187 | CCNB2 | 2.94729142 | 5.81162556 | 1.97185304 |
| 23 | 188 | FMOD | 0.33346407 | 0.24429053 | 0.73258426 |
| 24 | 189 | SLC7A8 | 0.23327957 | 0.68038164 | 2.91659333 |
| 25 | 190 | E2-EPF | 2.50218494 | 4.49667635 | 1.79709992 |
| 26 | 191 | AGT | 0.38629467 | 0.52277847 | 1.35331525 |
| 27 | 192 | FHL2 | 0.31699809 | 0.39190285 | 1.23629407 |
| 28 | 193 | LDLC | 0.56234146 | 0.88888889 | 1.58069244 |
| 29 | 194 | MGC16824 | 0.51520913 | 0.67362665 | 1.30748198 |
| 30 | 195 | UGDH | 0.4487715 | 0.59229116 | 1.31980566 |
| 31 | 196 | MAD2L1 | 4.48217081 | 6.89647789 | 1.53864683 |
| 32 | 197 | DDB2 | 0.37904516 | 0.3243275 | 0.85564341 |
| 33 | 198 | OS4 | 0.64290847 | 0.50896135 | 0.79165444 |
| 34 | 199 | BCL2 | 0.37660415 | 0.26111358 | 0.69333698 |
| 35 | 200 | SEMA3C | 0.5199821 | 0.48877024 | 0.93997512 |
| 36 | 201 | DTR | 7.22480411 | 0.4189956 | 0.05799404 |
| 37 | 202 | GARP | 0.47456604 | 0.3525155 | 0.74281654 |
| 38 | 203 | ACK1 | 0.52564876 | 0.49278642 | 0.93748232 |
| 39 | 204 | EDG2 | 0.71655585 | 0.46969319 | 0.6554872 |
| 40 | 205 | RARRES3 | 0.24142196 | 1.41881212 | 5.87689745 |
| 41 | 206 | CCNH | 0.55809994 | 0.42039831 | 0.75326706 |
| 42 | 207 | PREP | 1.84855753 | 1.63361667 | 0.88372509 |
| 43 | 208 | COL11A1 | 0.6377322 | 30.5047541 | 47.8331723 |
| 44 | 209 | GALC | 0.50650838 | 0.63980608 | 1.26316978 |
| 45 | 210 | HMGCS2 | 0.04797018 | 0.03074921 | 0.64100686 |
| 46 | 211 | ZNF274 | 1.70500973 | 0.86640362 | 0.50815172 |
| 47 | 212 | TFF1 | 0.0321807 | 0.2064045 | 6.41392222 |
| 48 | 213 | RAD51 | 3.1036169 | 2.89007176 | 0.93119475 |
| 49 | 214 | ASNS | 3.60284107 | 2.12910917 | 0.59095284 |
| 50 | 215 | PCMT1 | 2.46691568 | 1.76150989 | 0.71405355 |
| 51 | 216 | ESR1 | 0.12287491 | 0.2490413 | 2.02678727 |
| 52 | 217 | ACAT1 | 0.51017664 | 0.39593742 | 0.7760791 |
| 53 | 218 | XPA | 0.51539825 | 0.52117332 | 1.01120505 |
| 54 | 219 | LAF4 | 0.23519327 | 0.35275966 | 1.49987143 |
| 55 | 220 | COL10A1 | 0.38555774 | 9.32859382 | 24.1950629 |
| 56 | 221 | KIAA1041 | 1.44589009 | 1.01679685 | 0.70323246 |
| 57 | 222 | PLA2G7 | 4.23491725 | 4.95203213 | 1.16933386 |
| 58 | 223 | GRP | 0.12594309 | 0.25636115 | 2.03553163 |
| 59 | 224 | CYP2B6 | 0.01213194 | 0.12755005 | 10.513574 |
| 60 | 225 | CHAD | 0.02707726 | 0.17583189 | 6.49371152 |
| 61 | 226 | GALNT10 | 0.32020561 | 0.93356021 | 2.91550231 |
| 62 | 227 | GADD45B | 0.51944741 | 0.22157381 | 0.42655678 |
| 63 | 228 | WBSCR20 | 1.61337697 | 2.19652173 | 1.36144358 |
| 64 | 229 | BTBD2 | 0.59662324 | 1.02610179 | 1.71984885 |
| 65 | 230 | PGR | 0.06700908 | 0.12481888 | 1.86271582 |
| 66 | 231 | TBPL1 | 1.71529386 | 1.53220024 | 0.89325816 |
| 67 | 232 | C4B | 0.12173232 | 0.37926849 | 3.11559395 |
| 68 | 233 | CCNG1 | 0.46882525 | 0.37588048 | 0.80174965 |
| 69 | 234 | PDHB | 0.48347992 | 0.82135629 | 1.69884261 |
| 70 | 235 | HNRPDL | 0.62657647 | 0.54249869 | 0.86581401 |
| 71 | 236 | TAF11 | 1.83477376 | 1.42164687 | 0.77483497 |
| 72 | 237 | AMACR | 0.61312794 | 0.84739097 | 1.38207854 |
| 73 | 238 | EMD | 1.6831552 | 1.40144514 | 0.83262978 |
| 74 | 239 | NR2F1 | 0.2644964 | 0.09725355 | 0.36769327 |
| 75 | 240 | HSF2 | 1.72328808 | 1.03289666 | 0.5993755 |
| 76 | 241 | SPG4 | 2.02820496 | 1.22197745 | 0.60249209 |
| 77 | 242 | TRIP11 | 0.63637488 | 0.86619209 | 1.36113495 |
| 78 | 243 | OCLN | 0.47955471 | 0.70987061 | 1.48027033 |
| 79 | 244 | CACNA1D | 0.16768932 | 0.44304396 | 2.64205236 |
| 80 | 245 | CYP2B7 | 0.01399196 | 0.13737489 | 9.81812983 |
| 81 | 246 | FHL1 | 0.30932043 | 0.03099618 | 0.10020734 |
| 82 | 247 | MSX2 | 0.26991798 | 0.51082405 | 1.89251586 |
| 83 | 248 | PAI-RBP1 | 2.81808253 | 1.95566986 | 0.69397182 |
| 84 | 249 | CLDN14 | 0.34578658 | 0.30319698 | 0.87683272 |
| 85 | 250 | ITPK1 | 0.59689657 | 0.52128465 | 0.87332492 |
| 86 | 251 | ERBB2 | 1.86323083 | 7.16756759 | 3.84684897 |
| 87 | 252 | TP53 | 0.51575976 | 1.18684511 | 2.30115879 |
| 88 | 253 | HSPA2 | 0.09735986 | 0.34190488 | 3.51176445 |
| 89 | 254 | LIG1 | 0.3244685 | 0.36453228 | 1.12347509 |
| 90 | 255 | GSS | 0.58258632 | 0.84095907 | 1.44349265 |
| 91 | 256 | PRO1843 | 0.57531505 | 0.51177072 | 0.88954864 |
| 92 | 257 | MKI67 | 2.0943328 | 2.19410145 | 1.04763744 |
| 93 | 258 | BIK | 0.50587875 | 1.55537704 | 3.0746044 |
| 94 | 259 | KIAA0225 | 2.13074615 | 2.13861404 | 1.00369255 |
| 95 | 260 | TNRC15 | 0.63566173 | 0.69130642 | 1.0875382 |
| 96 | 261 | SFRS5 | 0.55670226 | 0.25236203 | 0.45331597 |
| 97 | 262 | RPL17 | 0.67408803 | 0.65848911 | 0.97685923 |
| 98 | 263 | GNG12 | 0.39809519 | 0.35596632 | 0.89417388 |
| 99 | 264 | LAP1B | 0.59182478 | 0.87189088 | 1.47322468 |
| 100 | 265 | LOC253782 | 0.33656287 | 1.0069827 | 2.99196016 |
| 101 | 266 | COL5A1 | 0.48612506 | 1.91919073 | 3.94793618 |
| 102 | 267 | CXCL13 | 1.09334867 | 2.55193586 | 2.33405493 |
| 103 | 268 | TTS-2.2 | 0.52779839 | 0.24321886 | 0.46081774 |
| 104 | 269 | KIAA0056 | 2.15880901 | 2.32531026 | 1.07712643 |
| 105 | 270 | FLJ22642 | 0.50735263 | 0.47592636 | 0.93805833 |
| 106 | 271 | LOC113146 | 0.4322237 | 0.20955508 | 0.48483016 |
| 107 | 272 | GPR126 | 2.97045989 | 1.28374752 | 0.4321713 |
| 108 | 273 | PMSCL1 | 3.85379762 | 5.25959238 | 1.36478168 |
| 109 | 274 | KIAA0418 | 0.63562548 | 0.58234822 | 0.91618138 |
| 110 | 275 | SULF1 | 1.05390365 | 3.85641652 | 3.65917372 |
| 111 | 276 | KIAA0673 | 0.57391504 | 0.57797443 | 1.00707314 |
| 112 | 277 | FLJ10803 | 2.8794926 | 0.80518888 | 0.27962874 |
| 113 | 278 | DKFZp586M0723 | 0.13647343 | 0.11662161 | 0.85453708 |
| 114 | 279 | C4A | 0.17445163 | 0.36240753 | 2.07740986 |
| 115 | 280 | ZAP3 | 0.60561667 | 0.54605096 | 0.90164454 |
| 116 | 281 | NEK9 | 0.42385526 | 0.71295236 | 1.6820656 |
| 117 | 282 | FLJ13125 | 1.7456421 | 1.35110145 | 0.77389671 |
| 118 | 283 | FMO5 | 0.08559415 | 0.30218827 | 3.53047791 |
| 119 | 284 | COMP | 0.2912537 | 4.73047702 | 16.2417748 |
| 120 | 285 | CSPG2 | 0.59090269 | 1.88790387 | 3.19494885 |
| 121 | 286 | LOC151996 | 0.41338598 | 2.34521857 | 5.67319337 |
| 122 | 287 | TFAP2B | 0.43320817 | 1.34577659 | 3.10653554 |
| 123 | 288 | OR7E38P | 2.4721374 | 2.04397969 | 0.82680667 |
| 124 | 289 | RAB31 | 0.40394741 | 2.19420728 | 5.43191319 |
| 125 | 290 | HSPC126 | 1.62954666 | 1.26787014 | 0.77805083 |
| 126 | 291 | UMP-CMPK | 1.92778452 | 1.24300347 | 0.64478341 |
| 127 | 292 | FLJ22195 | 1.43061659 | 1.51916101 | 1.06189249 |
| 128 | 293 | DCTN4 | 0.50788607 | 0.54260141 | 1.06835262 |
| 129 | 294 | FLJ20273 | 0.38803157 | 0.89334309 | 2.30224333 |
| 130 | 295 | KIF4A | 2.22685745 | 3.35533346 | 1.50675718 |
| 131 | 296 | THTP | 0.58831486 | 0.8535722 | 1.45087649 |
| 132 | 297 | PLSCR4 | 0.3444877 | 0.14809284 | 0.42989295 |
| 133 | 298 | FLJ11323 | 2.11180669 | 1.12860006 | 0.53442394 |
| 134 | 299 | MGC11242 | 0.39970231 | 0.96317642 | 2.40973447 |
| 135 | 300 | CEGP1 | 0.06321053 | 0.22757341 | 3.6002451 |
| 136 | 301 | SRR | 0.43030252 | 0.50748029 | 1.17935701 |
| 137 | 302 | HSPC177 | 0.54280584 | 0.75044087 | 1.38252174 |
| 138 | 303 | MGC3103 | 2.49147139 | 2.67377209 | 1.0731699 |
| 139 | 304 | FLJ20641 | 2.19559981 | 2.13795703 | 0.97374623 |
| 140 | 305 | FLJ13646 | 0.50690215 | 0.68417519 | 1.34971847 |
| 141 | 306 | KCNK15 | 0.08400027 | 0.30393847 | 3.6183034 |
| 142 | 307 | RNASEL | 0.43951061 | 0.48409168 | 1.10143344 |
| 143 | 308 | CRSP6 | 1.57038515 | 1.63575579 | 1.04162714 |
| 144 | 309 | COL5A2 | 0.44650047 | 1.59810403 | 3.57917657 |
| 145 | 310 | LOC51218 | 0.59078156 | 1.08711676 | 1.84013321 |
| 146 | 311 | APBB2 | 0.34810181 | 0.3281072 | 0.94256105 |
| 147 | 312 | yy15c12.s1 | 1.37222353 | 1.42335867 | 1.03726444 |
| 148 | 313 | AD037 | 2.09401866 | 1.44748322 | 0.69124657 |
| 149 | 314 | FLJ20477 | 0.52024352 | 0.42892996 | 0.82447919 |
| 150 | 315 | MARKL1 | 1.86975496 | 1.64523021 | 0.87991755 |
| 151 | 316 | LUM | 0.81501967 | 1.26269875 | 1.54928623 |
| 152 | 317 | COL3A1 | 0.60780953 | 1.3093042 | 2.15413568 |
| 153 | 318 | COL1A1 | 0.55118736 | 1.72152105 | 3.1232956 |
| 154 | 319 | BF | 0.23831298 | 1.7123556 | 7.18532235 |
| 155 | 320 | ADAM12 | 0.53384591 | 0.70372001 | 1.31820811 |
| 156 | 321 | LOXL1 | 0.48175564 | 1.99702419 | 4.14530526 |
| 157 | 322 | CEACAM6 | 0.57151883 | 7.72858988 | 13.5228963 |
| 158 | 323 | MMP11 | 0.75362281 | 6.87206597 | 9.11870749 |
| 159 | 324 | MMP1 | 26.1407301 | 117.806871 | 4.50664042 |
| 160 | 325 | MMP13 | 0.24808412 | 2.09572957 | 8.4476569 |
| 161 | 326 | SERPINH1 | 1.28483815 | 2.27223116 | 1.76849603 |
| 162 | 327 | PITX1 | 1.54911156 | 16.9745142 | 10.9575802 |
| 163 | 328 | RAD52 | 0.66443667 | 1.71706792 | 2.58424617 |
| 164 | 329 | INHBA | 0.72936034 | 4.21043511 | 5.77277773 |
| 165 | 330 | CSPG2 | 0.77410378 | 1.86511138 | 2.40938157 |

TABLE 4a
Putative biological function of 165 marker genes

| | SEQ ID | | |
|---|---|---|---|
| SEQ ID NO: | NO: | | |
| (DNA | (Protein | | |
| Sequence) | Sequence) | Gene_Symbol | Gene Description |
| 1 | 166 | CTSB | wu69b10.x1 cathepsin B |
| 2 | 167 | SSR1 | SSR alpha subunit signal sequence receptor alpha (translocon-associated protein alpha) |
| | | | SSR alpha subunit signal sequence receptor, alpha (translocon-associated |
| 3 | 168 | STX8 | MSS1 proteasome (prosome macropain) 26S subunit ATPase 2 mammalian suppressor of sgv1; |
| | | | transactivation factor proteasome (prosome, macropain) 26S subunit, ATPase, 2 |
| 4 | 169 | KPNA2 | nuclear localization sequence receptor hSRP1alpha karyopherin alpha 2 (RAG cohort 1 |
| | | | importin alpha 1) karyopherin alpha 2 (RAG cohort 1, importin alpha 1) |
| 5 | 170 | CSE1L | brain cellular apoptosis susceptibility protein (CSE1) brain cellular apoptosis susceptibility |
| | | | protein (CSE1) d chromosome segregation 1 (yeast homolog)-like CSE1 chromosome |
| | | | segregation 1-like (yeast) |
| 6 | 171 | RHEB2 | D78132 ras-related GTP-binding protein Ras homolog enriched in brain 2 Rheb; ras-related |
| | | | GTP-binding protein Ras homologue enriched in brain; similar to rat Rheb gene ras-related |
| | | | GTP-binding protein |
| 7 | 172 | DKC1 | Cbf5p homolog (CBF5) dyskeratosis congenita 1 dyskerin nucleolar protein; |
| | | | similar to yeast Cbf5p Cbf5p homolog |
| 8 | 173 | IGFBP4 | df29g03.y1 insulin-like growth factor-binding protein 4 insulin-like growth factor |
| | | | binding protein 4 |
| 9 | 174 | SMC1L1 | KIAA0178 gene SMC1 (structural maintenance of chromosomes 1 yeast)-like 1 KIAA0178 |
| | | | similar to mitosis-specific chromosome segregation protein SMC1 of S. cerevisiae. SMC1 structural |
| | | | maintenance of chromosomes 1-like 1 (yeast) |
| 10 | 175 | PWP1 | IEF SSP 9502 nuclear phosphoprotein similar to S. cerevisiae PWP1 |
| 11 | 176 | HDAC2 | transcriptional regulator homolog RPD3 histone deacetylase 2 similar to yeast RPD3, |
| | | | encoded by GenBank Accession Number X78454 transcriptional regulator |
| | | | homolog RPD3 |
| 12 | 177 | PRKAB1 | 5-AMP-activated protein kinase beta-1 protein kinase AMP-activated beta 1 non-catalytic |
| | | | subunit protein kinase, AMP-activated, beta 1 non-catalytic subunit |
| 13 | 178 | IMPDH2 | (clone FFE-7) type II inosine monophosphate dehydrogenase (IMPDH2) gene exons |
| | | | 1-13 IMP (inosine monophosphate) dehydrogenase 2 NAD-dependent; differentiation; inosine |
| | | | monophosphate dehydrogenase; inosine-5′-monophosphate dehydrogenase; nucleotide |
| | | | biosynthesis; proliferation associated gene IMP (inosine monophosphate) dehydrogenase 2 |
| 14 | 179 | UBE2A | HUMHHR6A HHR6A (yeast RAD 6 homologue) ubiquitin-conjugating enzyme E2A |
| | | | (RAD6 homolog) |
| 15 | 180 | YR-29 | hypothetical protein clone YR-29 hypothetical protein |
| 16 | 181 | MUF1 | MUF1 protein MUF1 protein |
| 17 | 182 | MYO10 | KIAA0799 protein myosin X hg01449 cDNA clone for KIAA0799 has a 1204-bp insertion at |
| | | | position 373 of the sequence of KIAA0799. KIAA0799 protein |
| 18 | 183 | EGFR | HSEGFPRE precursor of epidermal growth factor receptor epidermal growth factor receptor |
| | | | (avian erythroblastic leukemia viral (v-erb-b) oncogene homolog) epidermal growth factor receptor; |
| | | | signal peptide epidermal growth factor receptor epidermal growth factor receptor |
| | | | (erythroblastic leukemia |
| 19 | 184 | IFRD1 | BAC clone RG163K11 from 7q31 interferon-related developmental regulator 1 nucleophosmin |
| | | | 1 (nucleolar phosphoprotein B23 numatrin) pseudogene 14 HTG similar to mouse interferon-related |
| | | | protein PC4; 96% identical to P19182 (PID: g135861); H_RG163K11.1 |
| 20 | 185 | CD2BP2 | zk74b08.r1 CD2 antigen (cytoplasmic tail)-binding protein 2 CD2 antigen (cytoplasmic tail) |
| | | | binding protein 2 |
| 21 | 186 | ARL3 | 48c8 ADP-ribosylation factor-like 3 EST |
| 22 | 187 | CCNB2 | DKFZp434B174 (from clone DKFZp434B174) cyclin B2 cyclins B2 |
| | | | hypothetical protein |
| 23 | 188 | FMOD | fibromodulin fibromodulin precursor fibromodulin Encodes only the most carboxy terminal |
| | | | 58 amino acids of fibromodulin. fibromodulin |
| 24 | 189 | SLC7A8 | SLC7A8 protein solute carrier family 7 (cationic amino acid transporter y+ system) |
| | | | member 8 solute carrier family 7 (cationic amino acid transporter, |
| 25 | 190 | E2-EPF | HUME2EPI ubiquitin carrier protein (E2-EPF) ubiquitin carrier protein |
| 26 | 191 | AGT | G angiotensinogen serine (or cysteine) proteinase inhibitor clade A (alpha-1 antiproteinase |
| | | | antitrypsin) member 8 angiotensinogen (serine (or cysteine) proteinase inhibitor, |
| 27 | 192 | FHL2 | heart protein (FHL-2) four and a half LIM domains 2 |
| 28 | 193 | LDLC | LDLC low density lipoprotein receptor defect C complementing |
| 29 | 194 | MGC16824 | hypothetical protein |
| 30 | 195 | UGDH | UDP-glucose dehydrogenase (UGDH) UDP-glucose dehydrogenase UDPGDH; |
| | | | NAD+-linked oxidoreductase UDP-glucose dehydrogenase |
| 31 | 196 | MAD2L1 | MAD2 protein MAD2 (mitotic arrest deficient yeast homolog)-like 1 MAD2 gene MAD2-like |
| | | | 1 MAD2 mitotic arrest deficient-like 1 (yeast) |
| 32 | 197 | DDB2 | HSU18300 damage-specific DNA binding protein p48 subunit (DDB2) damage-specific |
| | | | DNA binding protein 2 (48 kD) damage-specific DNA binding protein p48 subunit; implicated in |
| | | | Xeroderma pigmentosum group E DDBb p48 |
| 33 | 198 | OS4 | OS-4 protein (OS-4) conserved gene amplified in osteosarcoma |
| 34 | 199 | BCL2 | HUMBCL2A B-cell leukemia lymphoma 2 (bcl-2) proto-oncogene encoding |
| | | | bcl-2-alpha protein B-cell leukemialymphoma 2 (bcl-2) proto-oncogene encoding bcl-2-alpha |
| | | | proteind B-cell CLL/lymphoma 2 alternative splicing; bcl-2-alpha protein; proto-oncogene bcl2-alpha |
| | | | protein B-cell lymphoma protein 2 beta |
| 35 | 200 | SEMA3C | AB000220 semaphorin E sema domain immunoglobulin domain (Ig) short basic |
| | | | domain secreted (semaphorin) 3C semaphorin E sema domain, immunoglobulin domain (Ig), |
| | | | short basic domain, secreted, (semaphorin) 3C |
| 36 | 201 | DTR | heparin-binding EGF-like growth factor diphtheria toxin receptor (heparin-binding epidermal |
| | | | growth factor-like growth factor) heparin-binding EGF-like growth factor putative diphtheria toxin |
| | | | receptor (heparin-binding epidermal growth factor-like growth factor) |
| 37 | 202 | GARP | garp gene glycoprotein A repetitions predominantprecursor glycoprotein A repetitions |
| | | | predominant GARP gene; leucine-rich repeat containing protein glycoprotein A repetitions |
| | | | predominant precursor |
| 38 | 203 | ACK1 | HUMNRTYKIN activated p21cdc42Hs kinase (ack) activated p21cdc42Hs kinase |
| | | | putative activated p21cdc42Hs kinase |
| 39 | 204 | EDG2 | wc44d05.x1 endothelial differentiation lysophosphatidic acid |
| | | | G-protein-coupled receptor 2 EST |
| 40 | 205 | RARRES3 | retinoic acid receptor responder 3 (RARRES3) retinoic acid receptor responder |
| | | | (tazarotene induced) 3 putative class II tumor suppressor; growth inhibitory protein; tazarotene |
| | | | induced retinoic acid receptor responder 3 |
| 41 | 206 | CCNH | HSU11791 cyclin H cyclin H cyclin H |
| 42 | 207 | PREP | prolyl oligopeptidase prolyl endopeptidase prolyl oligopeptidase |
| | | | prolyl endopeptidase |
| 43 | 208 | COL11A1 | alpha-1 type XI collagen (COL11A1) collagen type XI alpha 1 alpha-1 type XI collagen; |
| | | | collagen; type XI collagen alpha-1 (type XI) collagen precursor collagen, type XI, alpha 1 |
| 44 | 209 | GALC | DNAgalactocerebrosidase galactosylceramidase (Krabbe disease) GALC |
| | | | galactocerebrosidase |
| 45 | 210 | HMGCS2 | 3-hydroxy-3-methylglutaryl coenzyme A synthase 3-hydroxy-3-methylglutaryl-Coenzyme |
| | | | A synthase 2 (mitochondrial) hydroxymethyl-CoA synthetase 3-hydroxy-3-methylglutaryl-Coenzyme |
| | | | A synthase 2 (mitochondrial) |
| 46 | 211 | ZNF274 | zinc finger protein zfp2 (zf2) KRAB zinc finger protein HFB101L zinc finger |
| | | | protein 274 |
| 47 | 212 | TFF1 | EST186646 trefoil factor 1 (breast cancer estrogen-inducible sequence expressed in) |
| | | | EST trefoil factor 1 (breast cancer, estrogen-inducible sequence |
| 48 | 213 | RAD51 | DKFZp564H1178_s1 RAD51 (S. cerevisiae) homolog (E coli RecA homolog) |
| | | | EST RAD51 homolog (RecA homolog, E. coli) (S. cerevisiae) |
| 49 | 214 | ASNS | asparagine synthetase asparagine synthetase asparagine synthetase |
| 50 | 215 | PCMT1 | carboxyl methyltransferase protein-L-isoaspartate (D-aspartate) O-methyltransferase |
| | | | carboxyl methyltransferase protein-L-isoaspartate (D-aspartate) O-methyltransferase |
| 51 | 216 | ESR1 | HSERR oestrogen receptor estrogen receptor 1 estrogen receptor; receptor; steroid hormone |
| | | | receptor oestrogen receptor |
| 52 | 217 | ACAT1 | MAT genemitochondrial acetoacetyl-CoA thiolase acetyl-Coenzyme A acetyltransferase 1 |
| | | | (acetoacetyl Coenzyme A thiolase) (ACAT1) nuclear gene encoding mitochondrial prote |
| 53 | 218 | XPA | HUMXPAC XPAC protein xeroderma pigmentosum complementation group A XPAC protein |
| | | | xeroderma pigmentosum, complementation group A |
| 54 | 219 | LAF4 | lymphoid nuclear protein (LAF-4) lymphoid nuclear protein related to AF4 |
| 55 | 220 | COL10A1 | COL10A1 genecollagen (alpha-1 type X) collagen type X alpha 1 (Schmid metaphyseal |
| | | | chondrodysplasia) collagen, type X, alpha 1(Schmid metaphyseal chondrodysplasia) |
| 56 | 221 | KIAA1041 | KIAA1041 protein KIAA1041 protein KIAA1041 protein |
| 57 | 222 | PLA2G7 | LDL-phospholipase A2 phospholipase A2 group VII (platelet-activating factor acetylhydrolase |
| | | | plasma) PAF- acetylhydrolase phospholipase A2, group VII (platelet-activating factor |
| | | | acetylhydrolase, plasma) |
| 58 | 223 | GRP | HUMGRP5E gastrin-releasing peptide gastrin-releasing peptide gastrin-releasing peptide |
| | | | pre-progastrin releasing peptide gastrin-releasing peptide |
| 59 | 224 | CYP2B6 | HUMCYP2BB cytochrome P450-IIB (hIIB1) cytochrome P450 subfamily IIB (phenobarbital- |
| | | | inducible) polypeptide 6 cytochrome P450 subfamily IIB (phenobarbital-inducible) cytochrome P450; |
| | | | cytochrome P450 IIB cytochrome P450-IIB cytochrome P450, subfamily IIB (phenobarbital-inducible) |
| 60 | 225 | CHAD | chondroadherin gene 5flanking region and chondroadherin precursor cartilage leucine-rich |
| | | | repeat protein chondroadherin |
| 61 | 226 | GALNT10 | DKFZp586H0623 (from clone DKFZp586H0623) hypothetical protein DKFZp586H0623 |
| | | | (DKF hypothetical protein DKFZp586H0623 similarity to N-acetylgalactosaminyltransferase,; |
| | | | The frame shift was determined manually hypothetical protein putative UDP-GalNAc:polypeptide |
| | | | N-acetylgalactosaminyltransferase |
| 62 | 227 | GADD45B | growth arrest and DNA-damage-inducible protein GADD45beta growth arrest and |
| | | | DNA-damage-inducible beta growth arrest and DNA-damage-inducible, beta |
| 63 | 228 | WBSCR20 | wh80b02.x1 putative methyltransferase |
| 64 | 229 | BTBD2 | zd42a12.s1 BTB (POZ) domain containing 2 hypothetical protein FLJ20386 EST |
| 65 | 230 | PGR | progesterone receptor |
| 66 | 231 | TBPL1 | DNA sequence from clone 73H22 on chromosome 6q23 TBP-like 1 HTG; CpG Island |
| | | | dJ73H22.1 (TBP-like protein) |
| 67 | 232 | C4B | RP1 and complement C4B precursor (C4B) genes complement component 4B |
| 68 | 233 | CCNG1 | cyclin G1 clone MGC: 6 |
| 69 | 234 | PDHB | pyruvate dehydrogenase (EC 1.2.4.1) beta subunit gene exons 1-10 pyruvate |
| | | | dehydrogenase E1-beta subunit d pyruvate dehydrogenase (lipoamide) beta |
| 70 | 235 | HNRPDL | A + U-rich element RNA binding factor for A + U-rich element RNA binding |
| | | | factord heterogeneous nuclear ribonucleoprotein D-like |
| 71 | 236 | TAF11 | wr91e02.x1 TATA box binding protein (TBP)-associated factor RNA polymerase II I |
| | | | 28 kD TAF11 RNA polymerase II, TATA box binding protein (TBP)-associated |
| 72 | 237 | AMACR | 2-methylacyl-CoA racemase alpha-methylacyl-CoA racemase d |
| | | | alpha-methylacyl-CoA racemase |
| 73 | 238 | EMD | EDMD gene emerin (Emery-Dreifuss muscular dystrophy) clone MGC: 21 emerin |
| | | | (Emery-Dreifuss muscular dystrophy) EDMD gene; emerin emerin |
| 74 | 239 | NR2F1 | V-Erba Related Ear-3 Protein nuclear receptor subfamily 2 group F member 1 |
| | | | nuclear receptor subfamily 2, group F, member 1 |
| 75 | 240 | HSF2 | HUMHSF2 heat shock factor 2 (HSF2) heat shock factor 2 (HSF2) d heat shock |
| | | | transcription factor 2 heat shock factor 2 HSF2 |
| 76 | 241 | SPG4 | KIAA1083 protein spastic paraplegia 4 (autosomal dominant spastin) KIAA1083 |
| | | | protein spastic paraplegia 4 (autosomal dominant; spastin) |
| 77 | 242 | TRIP11 | Golgi-associated microtubule-binding protein (GMAP-210) thyroid hormone |
| | | | receptor interactor 11 GMAP-210 gene; Golgi-associated microtubule-binding protein |
| | | | Golgi-associated microtubule-binding protein |
| 78 | 243 | OCLN | wr26e08.x1 tight junction protein occludin d occludin EST |
| 79 | 244 | CACNA1D | wt59c07.x1 calcium channel voltage-dependent L type alpha 1D subunit |
| | | | ESTs calcium channel, voltage-dependent, L type, alpha 1D |
| 80 | 245 | CYP2B7 | cytochrome P450-IIB (hIIB3) ds cytochrome P450, subfamily IIB |
| | | | (phenobarbital-inducible), |
| 81 | 246 | FHL1 | LIM protein SLIMMER LIM protein SLIMMER d four and a half LIM domains |
| | | | 1 skeletal and cardiac muscle SLIM isoform LIM protein SLIMMER |
| 82 | 247 | MSX2 | MSX-2 msh (Drosophila) homeo box homolog 2 msh homeo box homolog 2 |
| | | | (Drosophila) |
| 83 | 248 | PAI-RBP1 | DKFZp564M2423 (from clone DKFZp564M2423) Similar to DKFZP564M2423 |
| | | | protein clone MGC: 13 DKFZP564M2423 protein |
| 84 | 249 | CLDN14 | CLDN14 gene claudin 14 (CLDN14) d claudin 14 claudin-14; CLDN14 |
| | | | gene claudin-14 |
| 85 | 250 | ITPK1 | inositol 1 3 4-trisphosphate 5 6-kinase inositol 1 3 4-trisphosphate 56-kinase d |
| | | | inositol 1 3 4-triphosphate 5/6 kinase inositol 1,3,4-triphosphate 5/6 kinase |
| 86 | 251 | ERBB2 | tyrosine kinase-type receptor (HER2) v-erb-b2 avian erythroblastic leukemia |
| | | | viral oncogene homolog 2 (neuroglioblastoma derived oncogene homolog) v-erb-b2 avian |
| | | | erythroblastic leukemia viral oncogene homolog 2 (neuro/glioblastoma derived oncogene |
| | | | homolog) tyrosine kinase HER2 receptor v-erb-b2 avian erythroblastic leukemia viral |
| | | | oncogene homolog 2 (neuro/glioblastoma derived oncogene homolog) |
| 87 | 252 | TP53 | HSP53 p53 cellular tumor antigen p53 cellular tumor antigen d tumor protein p53 |
| | | | (Li-Fraumeni syndrome) antigen; tumor antigen p53 tumor antigen (aa 1-?) tumor protein p53 |
| 88 | 253 | HSPA2 | HUMHSPA2A heat shock protein HSPA2 gene heat shock protein d heat |
| | | | shock 70 kD protein 2 |
| 89 | 254 | LIG1 | DKFZp434N0910_s1 for membrane glycoprotein LIG-1d DKFZP586O |
| | | | 1624 protein EST |
| 90 | 255 | GSS | wt55b10.x1 (clone pGSH1) glutathione synthetase (gsh-s) d glutathione |
| | | | synthetase |
| 91 | 256 | PRO1843 | initiation factor 4B eukaryotic translation initiation factor 4B |
| 92 | 257 | MKI67 | HSMKI67 mki67a (long type) antigen of monoclonal antibody Ki-67 antigen |
| | | | identified by monoclonal antibody Ki-67 |
| 93 | 258 | BIK | HSU34584 Bcl-2 interacting killer (BIK) BCL2-interacting killer (apoptosis-inducing) |
| | | | Bik (Bcl-2 interacting killer); Bcl-2 homology 3 (BH3) domain Bik interacts with the survival |
| | | | proteins Bcl-2, Bcl-xL, EBV-BHRF1 and adenovirus E1B 19 kD; This protein is identical with |
| | | | that described by Robin Brown and colleagues (personal communication) which is a |
| | | | Human NBK apoptotic inducer protein, encoded by GenB Bik |
| 94 | 259 | KIAA0225 | KIAA0225 gene KIAA0225 protein |
| 95 | 260 | TNRC15 | KIAA0642 protein trinucleotide repeat containing 15 |
| 96 | 261 | SFRS5 | zc81g05.s1 splicing factor arginineserine-rich 5 ESTs |
| 97 | 262 | RPL17 | L23 putative ribosomal protein ribosomal protein L17 ribosomal protein |
| | | | putative ribosomal protein (AA 1-184) ribosomal protein L17 |
| 98 | 263 | GNG12 | DKFZp586B0918 (from clone DKFZp586B0918) DKFZp586B0918 |
| | | | Zp586B0918) |
| 99 | 264 | LAP1B | UI-H-BI0-aao-g-10-0-UI.s1 FLJ11551 fis clone HEMBA1002999 |
| | | | moderately similar to Rattus norvegicus lamina associated polypeptide 1C (LAP1C) |
| | | | mRN DKFZP586G011 protein |
| 100 | 265 | LOC253782 | DKFZp434B102 (from clone DKFZp434B102): FLJ21238 fis clone |
| | | | COL01115 Homo sapiens mRNA; cDNA DKFZp434B102 (from clone DKFZp434B102) |
| 101 | 266 | COL5A1 | pro-alpha-1 (V) collagen collagen type V alpha 1 |
| 102 | 267 | CXCL13 | B lymphocyte chemoattractant BLC small inducible cytokine B subfamily |
| | | | (Cys-X-Cys motif) member 13 (B-cell chemoattractant) small inducible cytokine B |
| | | | subfamily (Cys-X-Cys motif), |
| 103 | 268 | TTS-2.2 | clone 24519 unknown transport-secretion protein 2.2 |
| 104 | 269 | KIAA0056 | KIAA0056 gene KIAA0056 protein |
| 105 | 270 | FLJ22642 | we38g03.x1: FLJ22642 fis clone HSI06970 EST |
| 106 | 271 | LOC113146 | 47g10 ESTs |
| 107 | 272 | GPR126 | DNA sequence from clone 287G14 on chromosome 6q23.1-24.3. Contains |
| | | | a novel seven transmembrane domain protein gene and an exon similar to parts of BMP and |
| | | | Tolloid genes. Contains ESTs an STS and GSSs DNA sequence from clone 287G14 on |
| | | | chromosome 6q23.1-24.3. Contains a novel seven transmembrane domain protein gene |
| | | | and an exon similar to parts of BMP and Tolloid genes. Contains ESTs an STS and GS |
| | | | Human DNA sequence from clone 287G14 on chromosome 6q23.1-24.3. Contains a novel |
| | | | seven transmembrane domain protein gene and an exon similar to parts of BMP and Tolloid |
| | | | genes. Contains ESTs an STS and GSSs HTG; BMP; seven transmembrane domain; Tolloid |
| | | | supported by GENSCAN and FGENES dJ287G14.1 (exon of a yet unidentified gene, or part |
| | | | of a pseudogene?; similar to parts of BMP and Tolloid proteins) |
| 108 | 273 | PMSCL1 | tx67e10.x1 polymyositisscleroderma autoantigen 1 (75 kD) EST Weakly similar to |
| | | | JH0446 75K autoantigen - human□ [H. sapiens] |
| 109 | 274 | KIAA0418 | wi34b03.x1 KIAA0418 gene product EST |
| 110 | 275 | SULF1 | KIAA1077 protein KIAA1077 protein KIAA1077 protein |
| 111 | 276 | KIAA0673 | KIAA0673 protein for KIAA0673 proteind KIAA0673 protein |
| | | | KIAA0673 protein |
| 112 | 277 | FLJ10803 | ni36d11.s1 hypothetical protein FLJ10803 ESTs |
| 113 | 278 | DKFZp586M0723 | DKFZp586M0723 (from clone DKFZp586M0723) |
| | | | DKFZp586M0723 Zp586M0723) |
| 114 | 279 | C4A | RP1 and complement C4B precursor (C4B) genes complement component |
| | | | C4A d complement component 4B complement component 4A |
| 115 | 280 | ZAP3 | (clone zap3) of cds and unknown ge ZAP3 protein ORF; putative |
| 116 | 281 | NEK9 | Untitled hypothetical protein MGC16714 |
| 117 | 282 | FLJ13125 | FLJ13125 fis clone NT2RP3002877 |
| 118 | 283 | FMO5 | flavin-containing monooxygenase 5 (FMO5) FLJ12110 fis clone MAMMA1000020 |
| | | | highly similar to for flavin-containing monooxygenase 5 (FMO5 flavin containing monooxygenase |
| | | | 5 flavin-containing monooxygenase 5 flavin containing monooxygenase 5 |
| 119 | 284 | COMP | germline oligomeric matrix protein (COMP) cartilage oligomeric matrix protein |
| | | | (pseudoachondroplasia epiphyseal dysplasia 1 multiple) cartilage oligomeric matrix protein |
| | | | (pseudoachondroplasia, |
| 120 | 285 | CSPG2 | pgH3 proteoglycan PG-M(V3) chondroitin sulfate proteoglycan 2 (versican) PG-M; |
| | | | proteoglycan PG-M(V3); large chondroitin sulfate proteoglycan; pgH3; major extracellular matrix |
| | | | molecule proteoglycan PG-M(V3) |
| 121 | 286 | LOC151996 | zv97h07.s1 FLJ12280 fis clone MAMMA1001744 EST |
| 122 | 287 | TFAP2B | transcription factor AP-2 beta (activating enhancer-binding protein 2 beta) |
| | | | transcription factor AP-2 beta (activating enhancer binding |
| 123 | 288 | OR7E38P | OR7E12P pseudogene complete sequence olfactory receptor family 7 |
| | | | subfamily E member 38 pseudogene olfactory receptor family 7 subfamily E member 12 |
| | | | pseudogene olfactory receptor |
| 124 | 289 | RAB31 | low-Mr GTP-binding protein (RAB31) RAB31 member RAS oncogene family |
| | | | Low Mr GTP-binding protein of the Rab subfamily low-Mr GTP-binding protein Rab31 RAB31, |
| | | | member RAS oncogene family |
| 125 | 290 | HSPC126 | wq62d04.x1 HSPC126 protein |
| 126 | 291 | UMP-CMPK | ws85a09.x1 UMP-CMP kinase EST |
| 127 | 292 | FLJ22195 | DKFZp762L203_s1 hypothetical protein FLJ22195 Homo sapiens |
| | | | cDNA: FLJ22195 fis clone HRC01166 |
| 128 | 293 | DCTN4 | wz58c04.x1 dynactin p62 subunit dynactin 4 (p62) |
| 129 | 294 | FLJ20273 | nh92d01.s1 hypothetical protein EST |
| 130 | 295 | KIF4A | zh97c02.s1 kinesin family member 4A EST |
| 131 | 296 | THTP | yi24d06.r1 hypothetical protein MGC2652 ESTs |
| 132 | 297 | PLSCR4 | wk77f02.x1 phospholipid scramblase 4 EST |
| 133 | 298 | FLJ11323 | ac16g07.s1 hypothetical protein FLJ11323 EST |
| 134 | 299 | MGC11242 | zh46f04.r1 hypothetical protein MGC11242 ESTs |
| 135 | 300 | CEGP1 | wv11f12.x1 CEGP1 protein |
| 136 | 301 | SRR | wq60g02.x1 serine racemase Homo sapiens cDNA FLJ13107 fis clone |
| | | | NT2RP3002501 weakly similar to THREONINE DEHYDRATASE CATABOLIC |
| | | | (EC 4.2.1.16) EST |
| 137 | 302 | HSPC177 | wn81b08.x1 hypothetical protein CGI-34 protein hypothetical |
| | | | protein HSPC177 |
| 138 | 303 | MGC3103 | ws44f11.x1 hypothetical protein MGC3103 ESTs |
| 139 | 304 | FLJ20641 | qi31h03.x1 hypothetical protein FLJ20641 |
| 140 | 305 | FLJ13646 | tg49h03.x1 hypothetical protein FLJ13646 Homo sapiens cDNA FLJ13646 |
| | | | fis clone PLACE1011325 EST |
| 141 | 306 | KCNK15 | two pore potassium channel KT3.3 |
| 142 | 307 | RNASEL | ribonuclease L (2 5-oligoisoadenylate synthetase-dependent) ribonuclease |
| | | | L (2′,5′-oligoisoadenytate synthetase-dependent) |
| 143 | 308 | CRSP6 | C05931 cofactor required for Sp1 transcriptional activation subunit 6 (77 kD) |
| | | | EST cofactor required for Sp1 transcriptional activation, |
| 144 | 309 | COL5A2 | yl92e08.r1 collagen type V alpha 2 TRIAD3 protein EST |
| 145 | 310 | LOC51218 | wr52b07.x1 clone FLB4739 EST |
| 146 | 311 | APBB2 | DKFZp434E033 (from clone DKFZp434E033) FE65-like protein (hFE65L) |
| | | | Homo sapiens mRNA; cDNA |
| 147 | 312 | yy15c12.s1 | DKFZp434E033 (from clone DKFZp434E033) amyloid beta (A4) |
| | | | precursor protein-binding, family B, yy15c12.s1 ESTs |
| 148 | 313 | AD037 | FE65-LIKE 2 AD037 protein |
| 149 | 314 | FLJ20477 | zx56a06.r1 hypothetical protein FLJ20477 EST |
| 150 | 315 | MARKL1 | DKFZp761B169_s1 ESTs MAP/microtubule affinity-regulating |
| | | | kinase like 1 |
| 151 | 316 | LUM | lumican lumican lumican |
| 152 | 317 | COL3A1 | pro-alpha-1 type 3 collagen collagen type III alpha 1 (Ehlers-Danlos syndrome |
| | | | type IV autosomal dominant) COL3A1 gene; collagen; collagen alpha 1 type III; collagen type III |
| | | | prepro-alpha-1 type 3 collagen |
| 153 | 318 | COL1A1 | prepro-alpha1(I) collagen proalpha 1 (I) chain of type I procollagen |
| | | | (partial collagen type I alpha 1 alpha1(I)-collagen collagen, type I, alpha 1 |
| 154 | 319 | BF | complement factor B B-factor properdin complement factor; complement factor B |
| | | | B-factor, properdin |
| 155 | 320 | ADAM12 | meltrin-L precursor (ADAM12) a disintegrin and metalloproteinase domain 12 |
| | | | (meltrin alpha) (ADAM12) transcript variant a disintegrin and metalloproteinase domain 12 |
| | | | (meltrin alpha) |
| 156 | 321 | LOXL1 | lysyl oxidase-like protein gene lysyl oxidase-like 1 lysyl oxidase-like 1 |
| 157 | 322 | CEACAM6 | nonspecific crossreacting antigen carcinoembryonic antigen-related cell adhesion |
| | | | molecule 6 (non-specific cross reacting antigen) clone MGC: 104 nonspecific cross-reacting antigen |
| | | | ORF1 non-specific cross reacting antigen |
| 158 | 323 | MMP11 | stromelysin-3 matrix metalloproteinase 11 (stromelysin 3) |
| 159 | 324 | MMP1 | skin collagenase matrix metalloproteinase 1 (interstitial collagenase) |
| 160 | 325 | MMP13 | collagenase 3 matrix metalloproteinase 13 (collagenase 3) |
| 161 | 326 | SERPINH1 | colligin (a collagen-binding protein) serine (or cysteine) proteinase inhibitor |
| | | | clade H (heat shock protein 47) member 1 collagen-binding protein; colligin colligin serine |
| | | | (or cysteine) proteinase inhibitor, clade H (heat |
| 162 | 327 | PITX1 | hindlimb expressed homeobox protein backfoot (Bft) paired-like homeodomain |
| | | | transcription factor 1 paired-like homeodomain transcription factor 1 |
| 163 | 328 | RAD52 | DKFZp564I1922 (from clone DKFZp564I1922) adlican d DKFZP564I1922 |
| | | homolog | protein similarity to perlecan hypothetical protein |
| 164 | 329 | INHBA | erythroid differentiation protein (EDF) inhibin beta A (activin A activin AB alpha |
| | | | polypeptide) inhibin, beta A (activin A, activin AB alpha polypeptide) |
| 165 | 330 | CSPG2 | the chondroitin sulphate proteoglycan versican V1 splice-variant precursor |
| | | | peptide chondroitin sulfate proteoglycan 2 (versican) |

TABLE 4b
Putative biological function of 20 nonresponder marker genes

| | SEQ ID | | |
|---|---|---|---|
| SEQ ID NO: | NO: | | |
| (DNA | (Protein | | |
| Sequence) | Sequence) | Gene_Symbol | Gene Description |
| 472 | 492 | PRG1 | hematopoetic proteoglycan core protein proteoglycan 1 secretory granule haematopoetic proteoglycan core |
| | | | protein hematopoetic proteoglycan core protein (AA 1-158) proteoglycan 1, secretory granule |
| 473 | 493 | GBP1 | guanylate binding protein isoform I (GBP-2) guanylate binding protein 1 interferon-inducible 67 kD |
| | | | guanylate binding protein isoform I guanylate binding protein 1, interferon-inducible, 67 kD |
| 474 | 494 | ALEX2 | KIAA0512 protein KIAA0512 gene product ALEX2 KIAA0512 gene product KIAA0512 protein |
| | | | KIAA0512 gene product armadillo repeat protein ALEX2 |
| 475 | 495 | CD53 | CD53 glycoprotein CD53 antigen |
| 476 | 496 | VCAM1 | vascular cell adhesion molecule-1 (VCAM1) gene vascular cell adhesion molecule 1 |
| 477 | 497 | MAPT | HUMTAUA microtubule-associated protein tau microtubule-associated protein tau epitope |
| | | | microtubule-associated protein tau microtubule-associated protein tau, isoform 2 |
| 478 | 498 | EGR2 | early growth response 2 protein (EGR2) early growth response 2 (Krox-20 (Drosophila) homolog) EGR2 |
| | | | gene; early growth response protein early growth response 2 protein early growth response 2 (Krox-20 |
| | | | homolog, Drosophila) |
| 479 | 499 | TDO2 | tryptophan oxygenase (TDO) tryptophan 2 3-dioxygenase tryptophan 2,3-dioxygenase |
| 480 | 500 | ADAMDEC1 | disintegrin-protease disintegrin protease disintegrin; protease disintegrin protease |
| 481 | 501 | TFEC | TFEC isoform (or TFECL) transcription factor EC TFEC TFEC isoform (or TFECL) |
| 482 | 502 | BTF3 | Transcription Factor Btf3b basic transcription factor 3 |
| 483 | 503 | FLNB | yi17d08.r1 filamin B beta (actin-binding protein-278) Homo sapiens mRNA; cDNA DKFZp586J021 |
| | | | (from clone DKFZp586J021) EST filamin B, beta (actin binding protein 278) |
| 484 | 504 | TFRC | transferrin receptor transferrin receptor (p90 CD71) clone MGC: 31 transferrin receptor (p90 CD71) |
| | | | transferrin receptor put. transferrin receptor (aa 1-760) transferrin receptor (p90, CD71) |
| 485 | 505 | EIF4B | eukaryotic translation initiation factor 4B |
| 486 | 506 | MAPK3 | HSERK1 ERK1 protein serine threonine kinase ERK1 for protein serinethreonine kinas mitogen-activated |
| | | | protein kinase 3 erk1 gene; protein-serine/threonine kinase protein serine/threonine kinase |
| 487 | 507 | LOC161291 | DKFZp564D1462 (from clone DKFZp564D1462) DKFZp564D1462 Zp564D1462) |
| 488 | 508 | SLC1A1 | High affinity glutamate transporter, important for reuptake of glutamate and has a role in excitatory |
| | | | neurotransmission |
| 489 | 509 | MST4 | serinethreonine protein kinase MASK (LOC51765), mRNA. |
| 490 | 510 | BLAME | BCM-like membrane protein precursor (SBBI42), mRNA. |
| 491 | 511 | NME7 | NME7 |

TABLE 5a
Primer and Probe sequences

| | | | | | | | |
|---|---|---|---|---|---|---|---|
| SEQ ID | SEQ ID | SEQ ID | SEQ ID | | | | |
| NO: | NO: | NO: | NO: | Gene_ | | | |
| (DNA) | (Probe) | (Primer 1) | (Primer 2) | Symbol | Probe | Forward Primer | Reverse Primer |

| | | | | | | | |
|---|---|---|---|---|---|---|---|
| 4 | 331 | 332 | 333 | KPNA2 | TCCTGCCCTAAGAGCCATA | GAGCTTCTGAATTGCCAA | GAGTCTGTTCATCTGTAC |
| | | | | | GGGAA | TTGTG | CAGTGACA |
| 5 | 334 | 335 | 336 | CSE1L | CTGCAGCTGACAAAATTCC | GCATTCTTAGAACGCGGT | TTGGATGCAATCAGCTTC |
| | | | | | TGGGTTACTAGGT | TCA | TGA |
| 6 | 448 | 449 | 450 | RHEB2 | ATTATCCTTCGAAAAACAT | AGCTTTTTTGGAATCTTC | GCCCCGTCCATTTTTTCT |
| | | | | | CCACAGCAGTCTG | TGCTAAA | G |
| 7 | 337 | 338 | 339 | DKC1 | TCTCGCTTCCGCTTCGCAG | GCAGGTAGTTGCCGAAGC | TGGAGGAGTCTCGTCACT |
| | | | | | TTTTTG | A | TTCA |
| 8 | 340 | 341 | 342 | IGFBP4 | TCTCCATTAGGCACATTCA | GGGTGGGAAGAAAGAATG | ACCCAGGAAGCCCCTCAT |
| | | | | | GTCCACT | CAA | C |
| 11 | 343 | 344 | 345 | HDAC2 | CCAAAGGAACCAAATCAGA | CCAAGGACAACAGTGGTG | GAAATTGGTGAGACTGTC |
| | | | | | ACAGCTCA | AAAA | AAATTCAG |
| 12 | 346 | 347 | 348 | PRKAB1 | AGTCGCCACAGATGTACCC | TTCTGTATACGCAGCTCA | CTTCCGCTGACTCACAGC |
| | | | | | ACTAGCCC | GTTTCC | AA |
| 13 | 349 | 350 | 351 | IMPDH2 | AAGAGCTTGACCCAAGTCC | CACTCATGCCAGGACATT | CAAACTTAAGCTCCCCAG |
| | | | | | GAGCCAT | GGT | AGTACAT |
| 15 | 352 | 353 | 354 | YR-29 | CAAGAAAACCACCTAAATA | TGCTTTGTTGGAGATGGC | TTGAAACGCAAGCCCATT |
| | | | | | TGAAAGATTCATC | TTT | G |
| 22 | 355 | 356 | 357 | CCNB2 | AACTTAACTAAATTCATCG | TGGCCAAGAATGTGGTGA | TCAGGAGTTTGCTGCTTG |
| | | | | | CCATCAAGAA | AAG | CA |
| 23 | 358 | 359 | 360 | FMOD | AGAAGATCCCCCCAGTCAA | TCCTTGAGCTAGACCTCT | ACTCATTGATCCTATTGC |
| | | | | | CACCAACC | CCTACAA | CTTGGA |
| 24 | 361 | 362 | 363 | SLC7A8 | CATCCAACGCCGTCGCTGT | TGTCTTTGCCAATGTCGC | AACAGAAATGGGCATGAT |
| | | | | | GAC | TTA | CCA |
| 25 | 364 | 365 | 366 | E2-EPF | TCGGATGCCCAGCTCAGCC | TGCGTCAACGTGCTCAAG | GGCACTTGATGGTCAGCA |
| | | | | | G | AG | GTAC |
| 26 | 367 | 368 | 369 | AGT | AAAGTGAGACCCTCCACCT | GCTGATCCAGCCTCACTA | AGATCCTTGCAGCACCAG |
| | | | | | TGTCCAGGT | TGC | TTG |
| 27 | 370 | 371 | 372 | FHL2 | CATGCCATGCAGTGCGTTC | GTGTGCCCTGCTATGAGA | CCCCTCCCGTGGTGATG |
| | | | | | AG | AACA | |
| 29 | 373 | 374 | 375 | MGC16824 | AGCCAGGAGACGTACCTTT | GGGAGGACAACAGCGATG | CCCCGTAGAGGCTGTCGT |
| | | | | | ACCACATAGA | AG | T |
| 31 | 376 | 377 | 378 | MAD2L1 | CACAGCTACGGTGACATTT | AAATCCGTTCAGTGATCA | CAGATCAAATGAACAAGA |
| | | | | | CTGCCACTG | GACAGA | AACTTCCA |
| 32 | 379 | 380 | 381 | DDB2 | TCTCAGAATGCACAAAAAG | TGAACATGGACGGCAAAG | CCAATCACAGCATGGGTT |
| | | | | | AAAGTGACGCA | AG | CAG |
| 40 | 382 | 383 | 384 | RARRES3 | CCAAGCGCCGTGGCCA | CAGGTGGAAAAGGCCAAG | AAGAGCATCCAGCAACAA |
| | | | | | | GT | CCA |
| 43 | 385 | 386 | 387 | COL11A1 | TCTATACCATCCTTATTCA | GTGCCACCAACCCATTTT | GTATTTCCTAAATGGTAC |
| | | | | | AAACTTGCAT | G | CTGTATATGCA |
| 50 | 388 | 389 | 390 | PCMT1 | ACAGGCAATATCAATCTTC | CCCCAGGCGCTAATAGAT | CTGCTCCAACATTTGGTT |
| | | | | | CTCCGGGCT | CA | TCC |
| 51 | 391 | 392 | 393 | ESR1 | ATGCCCTTTTGCCGATGCA | GCCAAATTGTGTTTGATG | GACAAAACCGAGTCACAT |
| | | | | | | GATTAA | CAGTAATAG |
| 55 | 394 | 395 | 396 | COL10A1 | TCCCCCTGAAAAGTGAGCA | CAGATTTGAGCTATCAGA | AAATTCAAGAGAGGCTTC |
| | | | | | GCAACGTA | CCAACAA | ACATACG |
| 58 | 397 | 398 | 399 | GRP | CGTTCTGCAAGCATCAGTT | AGAGAAAAACAAAACCCC | GCACAAGGAAATCTTGTT |
| | | | | | CTACG | TAAGAGACT | GATGAT |
| 61 | 400 | 401 | 402 | GALNT10 | CCACAGCATGAAGGGCAAC | GCCCTGTCACGCTGTACG | TCTTGTCTTTGCGGTATT |
| | | | | | CAGC | A | TCCA |
| 65 | 403 | 404 | 405 | PGR | TTGATAGAAACGCTGTGAG | AGCTCATCAAGGCAATTG | ACAAGATCATGCAAGTTA |
| | | | | | CTCGA | GTTT | TCAAGAAGTT |
| 68 | 406 | 407 | 408 | CCNG1 | ATGAAGGTACAGCCCAAGC | GCTGTGAATTTACTGGAC | AAATAAAAGCAGCTCAGT |
| | | | | | ACCTTGGG | AGATTCC | CCAACA |
| 69 | 409 | 410 | 411 | PDHB | ATCCTGGCACAGATTTCAG | GAAGGAGGCTGGCCACAG | TTGAACGCAGGACCTTCC |
| | | | | | CTCCTACTCCA | T | AT |
| 74 | 412 | 413 | 414 | NR2F1 | TGTACAGAATATATCCACA | TAAAACAGAAGGAAACTA | CAGTCCACTTCCATATGT |
| | | | | | TCCGTCCACAATAAATCCT | ATGGACCTT | GTTGTTC |
| 81 | 415 | 416 | 417 | FHL1 | CACTTCACGCAATGCTTGG | TGCGTGACTTGCCATGAG | GGTAAGTGATTCCTCCAG |
| | | | | | CA | A | ATGTGA |
| 82 | 418 | 419 | 420 | MSX2 | CAAACAGCCCATTAAGTTC | CAGAAGGTAAAGCCATGT | GGGACAGATGGACAGGAA |
| | | | | | CCTGG | TTTGACT | GGT |
| 83 | 421 | 422 | 423 | PAI-RBP1 | CTGATGTGGATGACCCAGA | ACCGACAAGTCAAGTGCT | GGTTGTCTTATGGCATCC |
| | | | | | GGCATTCC | TCTG | AGTTAA |
| 92 | 424 | 425 | 426 | MKI67 | TTTCTGATTCTGCATGAGA | GAGAGCGGAGGGCAGAAG | GAGAGCGGAGGGCAGAAG |
| | | | | | ACCTTCGCA | A | A |
| 98 | 427 | 428 | 429 | GNG12 | CCCCACCCCTCTGCTGGTC | CCAGATGCCTTGGTCCAA | GCAGCTTATAGCACCAAC |
| | | | | | CTG | AG | ACGTT |
| 100 | 430 | 431 | 432 | LOC253782 | CCCAAAGTTTCATAAAGCC | AATGGAAAACAACCTCTG | TGTGGGCAAAGAGTTGAT |
| | | | | | CCTAAGCTCATGA | AGTTTGA | GAAA |
| 101 | 433 | 434 | 435 | COL5A1 | CTTCGTGAGTGTCCCGTGC | CTCGTACCTCAGCATGCC | GTGCCGAGGCGTAGATGA |
| | | | | | AC | ATT | AG |
| 104 | 436 | 437 | 438 | KIAA0056 | ACGTGCAGTCAGGTGTCTT | CATCGGAGTCGGAGCTTA | CTCGCCATTCGACTCTTG |
| | | | | | CATACA | GG | CT |
| 105 | 439 | 440 | 441 | FLJ22642 | AATTCTAATGTAGCAAAAC | TGAAACGATTAGCTGTAG | CAGTAGATTTACCACACA |
| | | | | | GTAACCA | CCAAATT | TATTGCATTTT |
| 106 | 442 | 443 | 444 | LOC113146 | AACATAGTTTTCCTATTTC | GGTGTACAAGTCGTTTTT | GCTAAGTGAGTAGGAAAC |
| | | | | | AGGCAGAGTGCGGTATATT | GGTATAACTTC | AGTGTTTCC |
| | | | | | C | | |
| 108 | 445 | 446 | 447 | PMSCL1 | TGTTTCTACACCTGTGCTA | TGAAGCAGAACCTCCTTC | TCCAATTTGGGCAGTTCC |
| | | | | | TGGACTC | AGAAG | A |
| 113 | 451 | 452 | 453 | DKFZp586 | CAGACTAGCCATGACTTGA | AAAGAGCGTATGAAAAGT | TGACAAACACGACATAAA |
| | | | | M0723 | ATGCCAGCA | ACGTTAGACTT | TAACACACA |
| 124 | 454 | 455 | 456 | RAB31 | TTCCCCTGAAGGATGCTAA | AACAAGTGCGACCTCTCA | AACCACGATGGCACCTAT |
| | | | | | GGAATACGCT | GATATT | GG |
| 128 | 457 | 458 | 459 | DCTN4 | CACCTCATCTAATATAAAA | TCAATACCTGCAGCTGGT | GGTGCATACTGACTAGCA |
| | | | | | AAGGCAA | GAAT | TTAAAATTT |
| 129 | 460 | 461 | 462 | FLJ20273 | TCATCCCCTGACTGTGTGA | GACCAAATGTAATTCGGA | GAAACTCTGTGACAATCC |
| | | | | | AAAAAGTA | TCAGATC | TTCACTAGA |
| 132 | 463 | 464 | 465 | PLSCR4 | TTTTGAAAGATCTCCACCA | CTTGCTTCCTCATTGACT | GGCTTGCTGTGTCTCTCT |
| | | | | | CAACGT | TCATGT | ATCTTG |
| 133 | 466 | 467 | 468 | FLJ11323 | CCGCCGCGTCCCGAACT | GTGCTGACGGGACCCTTC | ACGAGAGCGAAACTCCAT |
| | | | | | | T | TTG |
| 138 | 469 | 470 | 471 | MGC3103 | CCCTGACTTCCGCAACATG | GCTGGCTGACAACTTCAT | GATGGCATTGCGAGACAG |
| | | | | | ACGG | CCA | TGT |

TABLE 5b
Primer and Probe sequences

| SEQ ID | SEQ ID | SEQ ID | SEQ ID | | | | |
|---|---|---|---|---|---|---|---|
| NO: | NO: | NO: | NO: | Gene_ | | | |
| (DNA) | (Probe) | (Primer 1) | (Primer 2) | Symbol | Probe | Forward Primer | Reverse Primer |
| 472 | 512 | 513 | 514 | PRG1 | CCCTCATCCTGGTTCTGGA | TCGGCTTGTCCTGGCTCT | TGGCTCTCCGCGTAGGAT |
| | | | | | ATCCTCAG | T | AA |
| 473 | 515 | 516 | 517 | GBP1 | CTTGGCCAGACCAATGCCC | CAGAGTCTTAGGTAAAAG | TGTCCTTGATATTGGGAC |
| | | | | | A | TCTTGGGAAA | ATTGTAG |
| 474 | 518 | 519 | 520 | ALEX2 | TTTTACTGGTTCTTCTGAA | AATCGTGCTGCTTGGATA | CAAATAATAGAACAGTAG |
| | | | | | TTGACAGTAAACCTGTCC | GAAATA | GCCATTCATAA |
| 475 | 521 | 522 | 523 | CO53 | TTTCGCATAGCAACCCTCC | CAGCATCTTGCCCCTCAG | AATTGGAATGAAACCACA |
| | | | | | ACTTTTCG | A | GTCTTG |
| 476 | 524 | 525 | 526 | VCAM1 | AAATGCCCATCTATGTCCC | TCCCTGAATGTATTGAAC | TTCAGGCAGCAAGTTTTA |
| | | | | | TTGC | TTGGAA | CTTTGA |
| 477 | 527 | 528 | 529 | MAPT | ATGGCAGCAGTTCCAACCT | CCCTCTGCTCCACAGAAA | GGTCTGCAAAGTGGCCAA |
| | | | | | TCAGAACTCAATA | CC | AAT |
| 478 | 530 | 531 | 532 | EGR2 | TCCCAAGCCATAAAGTGCA | GGACAGCAAAAAGACAAG | CTGTACAATGTCCCCCAA |
| | | | | | CAT | CAAA | ATCA |
| 479 | 533 | 534 | 535 | TDO2 | ATTCACTGATGACCAAATG | CAGTTGCTGACTTCTCTT | ATTCTGTGCACCATGCAC |
| | | | | | GAGATATAACC | ATGGACAT | ACA |
| 480 | 536 | 537 | 538 | ADAMDEC1 | AGTATCTGAGTTCAAAATT | TCCCTCTGGCAGTTGTGT | TGCACGGCAAGATGTACT |
| | | | | | CCCAAAGGA | GA | GAA |
| 481 | 539 | 540 | 541 | TFEC | CAGCGCATATCAGGATCAT | AATCAAGGAGCTTGGCAC | GATGCTTTTAGAATGGTT |
| | | | | | TAGACTTT | TCTT | CCTTTGTT |
| 482 | 542 | 543 | 544 | BTF3 | TTCGGCCAGTCTCCTTAAA | ACCAGCTTGGTGCGGATA | GTGCTTTTCCATCCACAG |
| | | | | | CTAGTCA | GT | ATTG |
| 483 | 545 | 546 | 547 | FLNB | CAGCAAAGCTGGCTCCAAC | TGTGGGCCAGAAGAGTTC | CCCATGGACCCCGATCA |
| | | | | | ATGCTG | CT | |
| 484 | 548 | 549 | 550 | TFRC | AGCTCCGTGAGTGAACCAT | GCCTACCCATTCGTGGTG | TCCCTAGGAGGCCGTTTC |
| | | | | | CATTATAAACGTG | AT | C |
| 485 | 551 | 552 | 553 | EIF4B | CCCACCACTTGTAGGGGAC | CTCGATCTCAGAGCTCAG | GCATTCATCCCATCTACT |
| | | | | | TGCT | ACACA | TTATTTTCAT |
| 486 | 554 | 555 | 556 | MAPK3 | CAGTGGCCGAGGAGCCCTT | AGTACTATGACCCGACGG | TCAGCCGCTCCTTAGGTA |
| | | | | | CAC | ATGAG | GGT |
| 487 | 557 | 558 | 559 | LOC161291 | CACCAGCCACTTTGCTAAT | GAACGATGATCTTAAAGG | TCTTGCTGCAATGTAAAT |
| | | | | | TTCTT | CACAAA | CTGCTAT |
| 488 | 560 | 561 | 562 | SLC1A1 | AGAAAAAGAGCTTCCCCTA | TGGGTTGAACAAGCCACG | GTCGTGGGATTTACTCTG |
| | | | | | ACCTGGG | TT | CAACA |
| 489 | 563 | 564 | 565 | MST4 | TAAGTATCCCTATTTCTTA | AATGTTGAGACACCGTTT | GTAGAGTCAACTAAAGAT |
| | | | | | AGTTACGAGGA | TGCTT | CAAAATGTGAAAG |
| 490 | 566 | 567 | 568 | BLAME | ATCACCTTCCCCCAAGATT | CCCTTTCCCACACCCACT | GGGATGGTGCAAGCTGAC |
| | | | | | ACCTGA | T | A |
| 491 | 569 | 570 | 571 | NME7 | TTGAAATCTCAGCTATGCA | TCCTGATGGCTATCCGAG | CCTCAACATTAACCCGAT |
| | | | | | GATGTTC | ATG | CCA |

TABLE 6
Statistical relevance of 20 genes differentially in non-responders (NC) as
compared to responding tumors. (CR—complete responder to therapy)

| SEQ ID NO: | SEQ ID NO: | | | | |
|---|---|---|---|---|---|
| (DNA | (Protein | | | Welch-Test | Wilcoxon |
| Sequence) | Sequence) | Gene_Symbol | T-Test p-value | p-value | p-value |
| 472 | 492 | PRG1 | 0.0002116 | 0.0002631 | 0.0003108 |
| 473 | 493 | GBP1 | 0.0020070 | 0.0023060 | 0.0029530 |
| 474 | 494 | ALEX2 | 0.0003502 | 0.0012570 | 0.0001554 |
| 475 | 495 | CD53 | 0.0019770 | 0.0039540 | 0.0018650 |
| 476 | 496 | VCAM1 | 0.0010630 | 0.0010690 | 0.0018650 |
| 477 | 497 | MAPT | 0.0005838 | 0.0007540 | 0.0001554 |
| 478 | 498 | EGR2 | 0.0008870 | 0.0009158 | 0.0006216 |
| 479 | 499 | TDO2 | 0.0084350 | 0.0105000 | 0.0018650 |
| 480 | 500 | ADAMDEC1 | 0.0018700 | 0.0021870 | 0.0029530 |
| 481 | 501 | TFEC | 0.0085550 | 0.0155500 | 0.0010880 |
| 482 | 502 | BTF3 | 0.0001140 | 0.0001471 | 0.0003108 |
| 483 | 503 | FLNB | 0.0006050 | 0.0007720 | 0.0018650 |
| 484 | 504 | TFRC | 0.0005408 | 0.0010110 | 0.0010880 |
| 485 | 505 | EIF4B | 0.0013130 | 0.0013330 | 0.0006216 |
| 486 | 506 | MAPK3 | 0.0001388 | 0.0003527 | 0.0006216 |
| 487 | 507 | LOC161291 | 0.0015790 | 0.0031610 | 0.0006216 |
| 488 | 508 | SLC1A1 | 0.0000179 | 0.0000389 | 0.0001554 |
| 489 | 509 | MST4 | 0.0000888 | 0.0000904 | 0.0001554 |
| 490 | 510 | BLAME | 0.0048620 | 0.0081110 | 0.0029530 |
| 491 | 511 | NME7 | 0.0020950 | 0.0021980 | 0.0006216 |

1. Method for characterizing the state of a neoplastic disease in a subject, comprising
(i) determining the pattern of expression levels of at least 6, 8, 10, 15, 20, 30, or 47 marker genes, comprised in a group of marker genes consisting of SEQ ID NO:1 to 165, in a biological sample from said subject,
(ii) comparing the pattern of expression levels determined in (i) with one or several reference pattern(s) of expression levels,
(iii) characterizing the state of said neoplastic disease in said subject from the outcome of the comparison in step (ii).

2. Method for characterizing the state of a neoplastic disease in a subject, comprising
(i) determining the pattern of expression levels of at least 6, 8, 10, 15, 20, 30, 47 or 67 marker genes, comprised in a group of marker genes consisting of SEQ ID NO:1 to 165 and 472 to 491, in a biological sample from said subject,
(ii) comparing the pattern of expression levels determined in (i) with one or several reference pattern(s) of expression levels,
(iii) characterizing the state of said neoplastic disease in said subject from the outcome of the comparison in step (ii).

3. Method for detection, diagnosis, screening, monitoring, and/or prognosis of a neoplastic disease in a subject, comprising
(i) determining the pattern of expression levels of at least 1, 2, 3, 5, 10, 15, 20, 30, or 47 marker genes, comprised in a group of marker genes consisting of SEQ ID NOs:1 to 17, 19 to 33, 35 to 50, 52 to 64, 66 to 85, 88 to 91, and 93 to 165 in biological samples from said subject,
(ii) comparing the pattern of expression levels determined in (i) with one or several reference pattern(s) of expression levels,
(iii) detecting, diagnosing, screening, monitoring, and/or prognosing said neoplastic disease in said subject from the outcome of the comparison in step (ii).

4. Method for detection, diagnosis, screening, monitoring, and/or prognosis of a neoplastic disease in a subject, comprising
(i) determining the pattern of expression levels of at least 1, 2, 3, 5, 10, 15, 20, 30, 47, or 67 marker genes, comprised in a group of marker genes consisting of SEQ D NOs:1 to 17, 19 to 33, 35 to 50, 52 to 64, 66 to 85, 88 to 91, and 93 to 165 and 472 to 491 in biological samples from said subject,
(ii) comparing the pattern of expression levels determined in (i) with one or several reference pattern(s) of expression levels,
(iii) detecting, diagnosing, screening, monitoring, and/or prognosing said neoplastic disease in said subject from the outcome of the comparison in step (ii).

5. Method of any of claims 1 to 4, wherein said method comprises multiple determinations of a pattern of expression levels, at different points in time, thereby allowing to monitor the development of said neoplastic disease in said subject.

6. Method of claim 1 or 2, wherein said method comprises an estimation of the likelihood of success of a given mode of treatment for said neoplastic disease in said subject.

7. Method of claim 1 or 2, wherein said method comprises an assessment of whether or not the subject is expected to respond to a given mode of treatment for said neoplastic disease.

8. Method of claim 6 or 7, wherein a predictive algorithm is used.

9. Method of claim 8, wherein the predictive algorithm is a Support Vector Machine.

10. Method of any of claims 6 to 9, wherein said given mode of treatment
(i) acts on cell proliferation, and/or
(ii) acts on cell survival, and/or
(iii) acts on cell motility, and/or
(iv) is an anthracycline based mode of treatment, and/or
(v) comprises administration of epirubicin and/or cyclophoshamid.

11. Method of treatment for a subject afflicted with a neoplastic disease, comprising
(i) identifying the most promising mode of treatment with the method of claim 6 or 7,
(ii) treating said neoplastic disease in said patient by the mode of treatment identified in step (i).

12. Method of screening for subjects afflicted with a neoplastic disease, wherein a method of any of claims 1 to 4 is applied to a plurality of subjects.

13. Method of screening for substances and/or therapy modalities having curative effect on a neoplastic disease comprising
(i) obtaining a biological sample from a subject afflicted with said neoplastic disease,
(ii) assessing, from said biological sample, using the method of claim 6 or 7, whether said subject is expected to respond to a given mode of treatment for said neoplastic disease,
(iii) if said subject is expected to respond to said given mode of treatment, incubating said biological sample with said substance under said therapy modalities,
(iv) observing changes in said biological sample triggered by said test substance under said therapy modalities,
(v) selecting or rejecting said test substance and/or said therapy modalities, based on the observation of changes in said biological sample under (iv).

14. Method of screening for compounds having curative effect on a neoplastic disease comprising
(i) incubating biological samples or extracts of these with a test substance,
(ii) determining the pattern of expression levels of at least 1, 2, 3, 5, 10, 15, 20, 30, or 47 marker genes, comprised in a group of marker genes consisting of SEQ ID NO:1 to 17, 19 to 33, 35 to 50, 52 to 64, 66 to 85, 88 to 91, and 93 to 165 in said biological sample,
(iii) comparing the pattern of expression levels determined in (ii) with one or several reference pattern(s),
(iv) selecting or rejecting said test substance, based on the comparison performed under (iii).

15. Method of screening for compounds having curative effect on a neoplastic disease comprising
(i) incubating biological samples or extracts of these with a test substance,
(ii) determining the pattern of expression levels of at least 1, 2, 3, 5, 10, 15, 20, 30, 47, or 67 marker genes, comprised in a group of marker genes consisting of SEQ ID NO:1 to 17, 19 to 33, 35 to 50, 52 to 64, 66 to 85, 88 to 91, and 93 to 165 and 472 to 491 in said biological sample,
(iii) comparing the pattern of expression levels determined in (ii) with one or several reference pattern(s),
(iv) selecting or rejecting said test substance, based on the comparison performed under (iii).

16. Method of any of claims 1 to 15 wherein said marker genes are comprised in a group of marker genes listed in Table 2.

17. Method of any of claims 1 to 16, wherein the expression level is determined
(i) with a hybridization based method, or
(ii) with a hybridization based method utilizing arrayed probes, or
(iii) with a hybridization based method utilizing individually labeled probes, or
(iv) by real time real time PCR, or
(v) by assessing the expression of polypeptides, proteins or derivatives thereof, or
(vi) by assessing the amount of polypeptides, proteins or derivatives thereof.

18. Method of any of claims 1 to 17, wherein the neoplastic disease is breast cancer.

19. A kit comprising at least 6, 8, 10, 15, 20, 30, or 47 primer pairs and probes suitable for marker genes comprised in a group of marker genes consisting of
(i) SEQ ID NO:1 to SEQ ID NO:165, or
(iii) the marker genes listed in Table 2.

20. A kit comprising at least 6, 8, 10, 15, 20, 30, 47, or 67 primer pairs and probes suitable for marker genes comprised in a group of marker genes consisting of
(i) SEQ ID NO: 1 to SEQ ID NO: 165, and/or
(ii) SEQ ID NO:472 to SEQ ID NO:491, or
(iii) the marker genes listed in Table 2.

21. A kit comprising at least 6, 8, 10, 15, 20, 30, or 47 individually labeled probes, each having a sequence comprised in a group of sequences consisting of SEQ ID NO:331 to SEQ ID NO:471.

22. A kit comprising at least 6, 8, 10, 15, 20, 30, 47 or 67 individually labeled probes, each having a sequence comprised in a group of sequences consisting of SEQ ID NO:331 to SEQ ID NO:471 and SEQ ID NO:512 to 571.

23. A kit comprising at least 6, 8, 10, 15, 20, 30, or 47 arrayed probes, each having a sequence comprised in a group of sequences consisting of SEQ ID NO:331 to SEQ ID NO:471.

24. A kit comprising at least 6, 8, 10, 15, 20, 30, 47 or 67 arrayed probes, each having a sequence comprised in a group of sequences consisting of SEQ ID NO:331 to SEQ ID NO:471 and SEQ ID NO:512 to 571.
